(19) United States
(12) Patent Application Publication
(10) Pub. No.: US 2017/0334985 A1
(43) Pub. Date: Nov. 23, 2017

(54) ANTI-IL-13/IL-17 BISPECIFIC ANTIBODIES AND USES THEREOF

(71) Applicant: Genentech, Inc., South San Francisco, CA (US)

(72) Inventors: LAWREN WU, FOSTER CITY, CA (US); JOSEPH R. ARRON, SAN MATEO, CA (US); MICHAEL DILLON, SAN FRANCISCO, CA (US); DAVID F. CHOY, FAIRFIELD, CA (US); SUE SOHN, EL CERRITO, CA (US); CHRISTOPH SPIESS, LOS ALTOS, CA (US); WHITNEY SHATZ, SAN FRANCISCO, CA (US)

(73) Assignee: Genentech, Inc., South San Francisco, CA (US)

(21) Appl. No.: 15/441,059

(22) Filed: Feb. 23, 2017

Related U.S. Application Data
(63) Continuation of application No. 15/236,333, filed on Aug. 12, 2016, now abandoned.
Application No. 15/236,333 is a continuation of application No. PCT/US2015/017168, filed on Feb. 23, 2015.
(60) Provisional application No. 61/942,823, filed on Feb. 21, 2014.
(60) Provisional application No. 61/983,945, filed on Apr. 24, 2014.

(51) Int. Cl. C07K 16/24; A61K 39/395; A61K 47/68; A61K 9/00; A61K 31/56; A61K 39/00

(52) U.S. Cl. CPC C07K 16/244; A61K 9/007; A61K 31/56; A61K 39/3955; A61K 47/6845; C07K 2317/31; C07K 2317/56; C07K 2317/76; C07K 2317/92; A61K 2039/505

(57) ABSTRACT

The invention provides anti-IL-13/IL-17 bispecific antibodies, in particular, anti-IL13/IL17 AA, AF and FF antibodies and methods of using the same, including without limitation, methods of using the anti-IL-13/IL-17 bispecific antibodies for treating moderate to severe asthma and/or eosinophilic asthma.

### CROSS REFERENCE TO RELATED APPLICATIONS

This application is a continuation of U.S. Ser. No. 15/236,333, filed Aug. 12, 2016, which is a continuation of International Application No. PCT/US2015/017168 having an international filing date of Feb. 23, 2015, the entire contents of which are incorporated herein by reference, and which relates to and claims the benefit of priority under 35 U.S.C. §119 to U.S. Provisional Application No. 61/942,823 filed Feb. 21, 2014 and U.S. Provisional Application No. 61/983,945 filed Apr. 24, 2014. The content of each of the provisional applications is herein incorporated by reference in its entirety.

### SEQUENCE LISTING

The instant application contains a Sequence Listing submitted via EFS-Web and hereby incorporated by reference in its entirety. Said ASCII copy, created on Feb. 22, 2017, is named P05707US5SeqList.txt and is 226,425 bytes in size.

### FIELD

The present invention relates to anti-IL-13/IL-17 bispecific antibodies, compositions comprising the bispecific antibodies and methods of using the same.

### BACKGROUND

Asthma is a complex disease with increasing worldwide incidence. Among other events, eosinophilic inflammation has been reported in the airways of asthma patients. The pathophysiology of the disease is characterized by variable airflow obstruction, airway inflammation, mucus hypersecretion, and subepithelial fibrosis. Clinically, patients may present with cough, wheezing, and shortness of breath. While many patients are adequately treated with currently available therapies, some patients with asthma have persistent disease despite the use of current therapies.

A number of studies have implicated IL-13, and its receptors in the pathogenesis of asthma and allergy (see, e.g., Wills-Karp, 2004, Immunol. Rev. 202, 175-190; Brightling et al., 2010, Clin. Exp. Allergy 40, 42-49; Finkelman et al., 2010, J Immunol 184, 1663-1674; Maes et al., 2012, Am. J. Respir. Cell Mol. Biol. 47, 261-270; Steinke and Borish, 2001, Respir. Res. 2, 66-70). IL-13 binds to two receptors, one a heterodimer of IL-4 receptor alpha (IL-4Rα) and IL-13 receptor alpha 1 (IL-13Rα1), and the other a single chain receptor consisting of IL-13 receptor alpha 2 (IL-13Rα2). Polymorphisms of the IL-13 and IL-4Rα genes are associated with asthma and allergy, including features such as IgE levels, prevalence of atopy, and severity of asthma disease. In addition, expression of IL-13 and its receptors are increased in asthma and other allergic diseases. Moreover, neutralization or deficiency of IL-13 and its receptors ameliorates disease in preclinical models of asthma.

A number of drugs are on the market or in development for treating asthma. One of the numerous targets for asthma therapy is IL-13. IL-13 is a pleiotropic TH2 cytokine produced by activated T cells, NKT cells, basophils, eosinophils, and mast cells, and it has been strongly implicated in the pathogenesis of asthma in preclinical models. IL-13 antagonists, including anti-IL-13 antibodies, have previously been described. See, e.g., Intn'l Patent Application Pub. No. WO 2005/062967. Such antibodies have also been developed as human therapeutics. Recently, several studies have shown clinical activity of monoclonal antibodies against IL-13 in the treatment of asthma (See, e.g., Corren et al., 2011, N. Engl. J. Med. 365, 1088-1098; Gauvreau et al., 2011, Am. J. Respir. Crit. Care Med. 183, 1007-1014; Ingram and Kraft, 2012, J. Allergy Clin. Immunol. 130, 829-42; Webb, 2011, Nat Biotechnol 29, 860-863). Of these, lebrikizumab, a humanized IgG4 antibody that neutralizes IL-13 activity, improved lung function in asthmatics who were symptomatic despite treatment with, for the majority, inhaled corticosteroids and/or a long-acting beta2-adrenergic receptor agonist (Corren et al., 2011, N. Engl. J. Med. 365, 1088-1098).

Growing evidence has shown that asthma is a heterogeneous disease that may implicate multiple pathways. For example, the expression of IL-17A and IL-17F was found to be associated with severe asthma. In addition, both IL-17A and IL-17F have been implicated as contributing agents to progression and pathology of a variety of inflammatory and auto-immune diseases in humans and in mouse models of human diseases. Specifically, IL-17A and IL-17F have been implicated as major effector cytokines that trigger inflammatory responses and thereby contribute to a number of autoinflammatory diseases.

IL-17A (originally named CTLA-8, sometimes referred to in the field as IL-17) is the archetypical/founding member of the IL-17 family of cytokines. In addition to IL-17A, members of the IL-17 cytokine family presently include the proteins IL-17B, IL-17C, IL-17D, IL-17E (also called IL-25) and IL-17F that share a conserved C-terminal region but differ in their N-terminal segments.

IL-17A and IL-17F are the two most closely related members of the family, both in terms of sequence and biological properties. IL-17F shares 55% sequence identity with IL-17A at the amino acid level. Both IL-17A and IL-17F are secreted as disulfide linked homodimers or as a heterodimer, which signal through a heterodimeric receptor comprised of IL-17RA and IL-17RC. The IL-17 receptor is expressed on various T cell subsets (such as Th17 CD4+ T cells).

Despite the suggestion that IL-17 may play a role in asthma, individual contributions of IL-17A homodimer, IL-17F homodimer or IL-17A/F heterodimers in airway hyper-responsiveness remain unclear. McKinley et al., 2008, J. Immunol., 181:4089. Clinical trials of therapeutic antagonist antibodies that target the IL-17 pathway for treating asthma have led to negative results. For example, Kirsten et al. reported that a therapeutic anti-IL-17A antibody could not demonstrate a treatment effect on ozone-induced airway neutrophilia in healthy volunteers, a model of neutrophilic airway inflammation for testing the safety and efficacy of ant-inflammatory drugs in early development. Kirsten et al. 2013, European Respiratory Journal, 41:239, Scheerens et al., 2013, Clinical & Experimental Allergy 44:38-46. In addition, recent clinical trial results showed that a therapeutic anti-IL-17RA human antibody brodalumab did not produce a treatment effect in subjects with moderate to severe asthma in a randomized, double-blind, placebo-controlled study. See Busse et al., 2013, Am. J. Respir. Crit. Care Med. 188:1294-1302. A recent clinical review article referred to the same brodalumab study and reported that even subphenotyping by the presence of blood neutrophils or eosinophils did not better identify a responder group. Fajt et al., J. Allergy Clin. Immunology, 2014, 135:299. IL-17RA is not only a receptor component for IL-17AA, FF and AF heterodimer, but also a receptor component for IL-25, which plays an important role in TH2 inflammation and induces IL-13 expression. See Tamachi et al. 2006, Intl. Archives Allergy and Imunol. 140 (suppl 1):59. Thus, it is uncertain whether blockade of the IL-17A and F pathways can lead to an effective treatment of asthma.

WO 2013/102042 describes several dual variable domain (DVD) antibodies targeting IL-13 and IL-17A and characterizes the affinities and in vitro neutralization activities of the anti-IL-13/IL-17 DVD antibodies. WO 2013/102042 proposes that the DVD bispecific antibodies can be used for treating a variety of diseases, for example, infectious diseases, autoimmune diseases, asthma, Rheumatoid arthritis, systemic lupus erythematosus, multiple sclerosis, sepsis, neurologicl disorders, spinal cord injury, and oncology disorders. No preclinical results or clinical efficacy, however, has been demonstrated either by using any one of the anti-IL-13/IL-17 DVD antibodies or by proving the concept of targeting both IL-13 and IL-17, in these diseases.

Therefore, moderate to severe asthmatic patients are still in need of alternative treatment options. Thus, there is a need to identify better therapies for treating asthma and improved methods for understanding how to treat asthma patients.

Another inflammation disease in the airway idiopathic pulmonary fibrosis (IPF) is a restrictive lung disease characterized by progressive interstitial fibrosis of lung parenchyma, affecting approximately 100,000 patients in the United States (Raghu et al., Am J Respir Crit Care Med 174:810-816 (2006)). This interstitial fibrosis associated with IPF leads to progressive loss of lung function, resulting in death due to respiratory failure in most patients. The median survival from the time of diagnosis is 2-3 years (Raghu et al., Am J Respir Crit Care Med 183:788-824 (2011)). The etiology and key molecular and pathophysiological drivers of IPF are unknown. The only treatment shown to prolong survival in IPF patients is lung transplantation (Thabut et al., Annals of internal medicine 151:767-774 (2009)). Lung transplantation, however, is associated with considerable morbidity. Further, not all IPF patients are appropriate candidates for transplantation, and there is a relative paucity of suitable donor lungs. Despite numerous attempts, no drug therapies to date have been shown to substantially prolong survival in a randomized, placebo-controlled interventional trial in IPF patients, although some interventions have appeared to slow the rate of lung function decline in some patients (Raghu et al., Am J Respir Crit Care Med 183:788-824 (2011); Richeldi et al., The New England J. of Med. 365:1079-1087 (2011)).

IPF patients are still in need of alternative treatment options. Thus, there is a need to identify better therapies for treating IPF and improved methods for understanding how to treat IPF patients

All references cited herein, including patent applications and publications, are incorporated by reference herein in their entirety for any purpose.

### SUMMARY

In one aspect, the invention provides methods of treatment using anti-IL-13/IL-17 multispecific antibodies, in particular, bispecific antibodies that bind and inhibit IL-13 and IL-17AA, AF and FF, and the anti-IL-13/IL-17 multispecific antibodies. The invention described herein is partly based on the discovery of an improved therapy for asthma using the anti-IL-13/IL-17 bispecific antibodies. In certain embodiments, the asthma is eosinophilic asthma. In certain embodiments, the anti-IL-13/IL-17 bispecific antibody retains the wild-type full-length antibody format, but differs from the wild-type monospecific bivalent antibody in that the bispecific antibody is a monovalent binder to each target. Yet, the bispecific antibody maintains comparable affinity and potency with regard to each target as compared to each of the parent monospecific bivalent antibody. In certain embodiments, the invention also relates to the surprising findings that the anti-IL-13/IL-17 bispecific antibody was difficult to make as further described herein.

In one aspect of the invention, the antibodies described herein are provided for use as a medicament. In some embodiments, the antibodies described herein are provided for use in the preparation of a medicament for treating an eosinophilic disorder, an IL-13 mediated disorder, an IL-17 mediated disorder, and/or a respiratory disorder. In some embodiments, the antibodies described herein are provided for use in treating an eosinophilic disorder, an IL-13 mediated disorder, an IL-17 mediated disorder, and/or a respiratory disorder. In some embodiments, use of the antibodies described herein in the manufacture of a medicament for treating an eosinophilic disorder, an IL-13 mediated disorder, an IL-17 mediated disorder, and/or a respiratory disorder is provided. In some embodiments, methods of treating an eosinophilic disorder, an IL-13 mediated disorder, an IL-17 mediated disorder, and/or a respiratory disorder in an individual are provided comprising administering to the individual an effective amount of an antibody described herein. In certain embodiments, the antibodies described herein are provided for use in treating an eosinophilic disorder and a neutrophilic disorder. In certain embodiments, the antibodies described herein are provided for use in treating eosinophilic asthma, neutrophilic asthma, eosinophilic and neutrophilic asthma, mixed asthma and/or mixed granulocytic asthma. In certain embodiments, methods of treating an eosinophilic disorder and a neutrophilic disorder are provided comprising administering to an individual in need thereof an effective amount of an antibody described herein. In certain embodiments, methods of treating eosinophilic asthma, neutrophilic asthma, eosinophilic and neutrophilic asthma, mixed asthma and/or mixed granulocytic asthma are provided comprising administering to an individual in need thereof an effective amount of an antibody described herein.

In certain embodiments, a patient suffering from an eosinophilic inflammation or disorder may exhibit elevated level of one or more of the eosinophilic signature genes. In certain embodiments, the patient is identified as an Eosinophilic Inflammation Positive (EIP) patient that shows elevated serum periostin levels and/or elevated levels of one or more selected from CSF1 (macrophage colony stimulating factor 1, Entrez ID 1435), MEIS2 (Meis homeobox 2, Entrez ID 4212), LGALS12 (lectin, galactoside-binding, soluble, 12, Entrez ID 85329), IDO1 (indoleamine 2,3-dioxygenase 1, Entrez ID 3620), THBS4 (thrombospondin 4, Entrez ID 7060), OLIG2 (oligodendrocyte lineage transcription factor 2, Entrez ID 10215), ALOX15 (arachidonate 15-lipoxygenase, Entrez ID 246), SIGLEC8 (sialic acid binding Ig-like lectin 8, Entrez ID 27181), CCL23 (chemokine (C—C motif) ligand 23, Entrez ID 6368), PYROXD2 (pyridine nucleotide-disulphide oxidoreductase domain 2, Entrez ID 84795), HSD3B7 (hydroxy-delta-5-steroid dehydrogenase, 3 beta- and steroid delta-isomerase 7, Entrez ID 80270), SORD (sorbitol dehydrogenase, Entrez ID 6652), ASB2 (ankyrin repeat and SOCS box containing 2, Entrez ID 51676), CACNG6 (calcium channel, voltage-dependent, gamma subunit 6, Entrez ID 59285), GPR44 (G protein-coupled receptor 44, Entrez ID 11251), MGAT3 (mannosyl (beta-1,4-)-glycoprotein beta-1,4-N-acetylglucosaminyltransferase, Entrez ID 4248), SLC47A1 (solute carrier family 47, member 1, Entrez ID 55244), SMPD3 (sphingomyelin phosphodiesterase 3, neutral membrane, Entrez ID 55512), CCR3 (chemokine (C—C motif) receptor 3, Entrez ID 1232), CLC (Charcot-Leyden crystal protein, Entrez ID 1178), CYP4F12 (cytochrome P450, family 4, subfamily F, polypeptide 12, Entrez ID 66002), and ABTB2 (ankyrin repeat and BTB (POZ) domain containing 2, Entrez ID 25841), as compared to a control. Alternatively or additionally, the patient may exhibit elevated levels of one or more of the neutrophilic signature genes such as CXCR1, CXCR2, neutrophil elastase, or CEACAM6. Accordingly, in certain embodiments, the methods provided herein further comprise the step of measuring in the patient the levels of serum periostin and/or one or more of the eosinophil signature genes or one or more of neutrophil signature genes. In certain embodiments, the methods provided herein further comprise the step of measuring in the patient the levels of serum periostin. In certain embodiments, the serum periostin is Total Periostin. In certain embodiments, the methods provided herein further comprise the step of measuring in the patient blood eosinophil counts. In certain embodiments, the methods provided herein further comprise the step of measuring in the patient blood neutrophil counts.

In some such embodiments, a method further comprises administering to the individual a TH2 pathway inhibitor. In some embodiments, the TH2 pathway inhibitor inhibits at least one target selected from ITK, BTK , IL-9, IL-5, IL-13, IL-4, OX40L, TSLP, IL-25, IL-33, IgE, IL-9 receptor, IL-5 receptor, IL-4 receptor alpha, IL-13receptoralpha1 (IL-13Rα1), IL-13receptoralpha2 (IL-13Rα2), OX40, TSLP-R, IL-7Ralpha, IL-17RB, ST2, CCR3, CCR4, CRTH2, FcepsilonRI, FcepsilonRII/CD23, Flap, Syk kinase; CCR4, TLR9, CCR3, IL5, IL3, and GM-CSF. In some embodiments, a method further comprises administering to the individual a TH17 pathway inhibitor. In some embodiments, the TH17 pathway inhibitor inhibits at least one target selected from IL-1β, IL-6, IL-17A homodimer, IL-17F homodimer, IL-17AF heterodimer, IL-22, IL-21, TGF-β, IL-23, IL-26, IL-10 receptor, IL-6 receptor, IL-17 receptor, IL-17RA, IL-17RC, IL-22R1, IL10R2, IL-21 receptor, TGF-β receptor, IL-26 receptor and IL-23 receptor (IL-12Rb1, IL23R). In some embodiments, methods of treating moderate to severe asthma are provided. In some embodiments, methods of treating idiopathic pulmonary fibrosis are provided. In certain embodiments, methods of treating atopic dermatitis are provided. In some embodiments, methods of treating an individual with high serum periostin are provided. In some embodiments, methods of treating periostin-high asthma are provided.

In any of the embodiments described herein, the eosinophilic disorder may be selected from asthma (including aspirin sensitive asthma), atopic asthma, atopic dermatitis, allergic rhinitis (including seasonal allergic rhinitis), non-allergic rhinitis, asthma, severe asthma, chronic eosinophilic pneumonia, allergic bronchopulmonary aspergillosis, coeliac disease, Churg-Strauss syndrome (periarteritis nodosa plus atopy), eosinophilic myalgia syndrome, hypereosinophilic syndrome, oedematous reactions including episodic angiodema, helminth infections, onchocercal dermatitis and Eosinophil-Associated Gastrointestinal Disorders, eosinophilic esophagitis, eosinophilic gastritis, eosinophilic gastroenteritis, eosinophilic enteritis, eosinophilic colitis, nasal micropolyposis and polyposis, aspirin intolerance, asthma and obstructive sleep apnoea, chronic asthma, Crohn's disease, scleroderma and endomyocardial fibrosis, cancer (e.g., glioblastoma (such as glioblastoma multiforme), non-Hodgkin's lymphoma (NHL)), atopic dermatitis, allergic rhinitis, asthma, fibrosis, inflammatory bowel disease, pulmonary fibrosis (including idiopathic pulmonary fibrosis (IPF) and pulmonary fibrosis secondary to sclerosis), COPD, and hepatic fibrosis. In some embodiments, the IL-13 mediated disorder is selected from atopic dermatitis, allergic rhinitis, asthma, fibrosis, inflammatory bowel disease, Crohn's disease, a respiratory disorder, lung inflammatory disorders, pulmonary fibrosis, idiopathic pulmonary fibrosis (IPF), chronic obstructive pulmonary disease (COPD), hepatic fibrosis, cancer, glioblastoma, and non-Hodgkin's lymphoma. In some embodiments, the neutrophilic disorder or IL-17 mediated disorder may be selected from atopic dermatitis, allergic rhinitis, asthma, fibrosis, inflammatory bowel disease, Crohn's disease, lung inflammatory disorders, pulmonary fibrosis, idiopathic pulmonary fibrosis (IPF), chronic obstructive pulmonary disease (COPD), hepatic fibrosis, a respiratory disorder, cancer, glioblastoma, and non-Hodgkin's lymphoma. In any of the embodiments described herein, the respiratory disorder may be selected from asthma, allergic asthma, non-allergic asthma, bronchitis, chronic bronchitis, chronic obstructive pulmonary disease (COPD), emphysema, cigarette-induced emphysema, airway inflammation, cystic fibrosis, pulmonary fibrosis, allergic rhinitis, and bronchiectasis.

In some embodiments, a multispecific antibody described herein is provided for use in treating asthma or a respiratory disorder. In some embodiments, the asthma is moderate to severe asthma. In certain embodiments, the asthma is TH2 high asthma. In certain embodiments, the asthma is Th2-driven asthma. In certain other embodiments, the asthma is eosinophilic asthma. In certain embodiments, the asthma is allergic asthma. In some embodiments, the individual has been determined to be Eosinophilic Inflammation Positive (EIP). In certain embodiments, the individual has been determined to have elevated levels of at least one of the eosinophilic signature genes as compared to a control or reference level. In certain embodiments, the asthma is periostin-high asthma. In certain embodiments, the asthma is eosinophil-high asthma. In some embodiments, the individual has high serum periostin. In certain embodiments, the individual is eighteen year or older. In certain embodiments, the individual has been determined to have an elevated level of serum periostin as compared to a control or reference level. In certain embodiments, the individual has been determined to have 20 ng/ml or more serum periostin. In certain embodiments, the individual has been determined to have 25 ng/ml or more serum periostin. In certain embodiments, the individual has been determined to have 50 ng/ml or more serum periostin. In certain embodiments, the control or reference level of serum periostin is 20 ng/ml, 25 ng/ml or 50 ng/ml. In certain embodiments, the serum periostin is Total Periostin. In certain embodiments, the individual has been determined to have an elevated level of blood eosinophil counts as compared to a control or reference count or level. In certain embodiments, the individual has been determined to have an elevated sputum eosinophil count as compared to a control or reference count or level. In certain embodiments, the individual has been determined to have at least 150, at least 200, at least 250, at least 300 or at least 400/ul. In some embodiments, the individual has elevated expression or levels of at least one, at least two, at least three, at least four, or all of the following genes: CXCL1, IL8, CXCL2, CXCL3, and CSF3, as compared to a control individual. In some embodiments, the individual has high serum periostin and elevated expression or levels of at least one, at least two, at least three, at least four, or all of the following genes: CXCL1, IL8, CXCL2, CXCL3, and CSF3, as compared to a control individual. In certain other embodiments, the individual has high serum periostin and high serum or plasma CXCL1. In certain other embodiments, the individual has high serum periostin and high serum or plasma IL8. In certain other embodiments, the individual has high serum periostin and high serum or plasma CXCL2. In certain other embodiments, the individual has high serum periostin and high serum or plasma CXCL3. In certain other embodiments, the individual has high serum periostin and high serum or plasma CSF3. In certain embodiments, the individual has been determined to have an elevated level of serum periostin and/or blood eosinophil count and/or blood neutrophil count, as compared to a control or reference level. In certain embodiments, the Total Periostin is measured or determined. In some embodiments, an elevated serum periostin level refers to at least 20 ng/ml, at least 25 ng/ml, at least 30 ng/ml, at least 40 ng/ml, or at least 50 ng/ml of Total Periostin. In certain embodiments, Total periostin is measured or determined by any methods known in the art, for example ELISA. In some embodiments, Total Periostin is determined by the E4 assay or the ELECSYS® periostin assay described herein.

In some embodiments, the asthma is uncontrolled on a corticosteroid. In some embodiments, the corticosteroid is an inhaled corticosteroid. In some embodiments, the inhaled corticosteroid is selected from beclomethasone dipropionate (e.g., Qvar®), budesonide (e.g., Pulmicort®), budesonide/formoterol fumarate dehydrate (e.g., Symbicort®), flunisolide (e.g., Aerobid®), fluticasone propionate (e.g., Flovent®, Flonase®), fluticasone propionate and salmeterol (e.g., Advair®), and triamcinolone acetonide (e.g., Azmacort®). In some embodiments, the individual is also being treated with a second controller. In some embodiments, the second controller is a long-acting bronchial dialator (LABD). In some embodiments, the LABD is selected from a long-acting beta-2 agonist (LABA), a leukotriene receptor antagonist (LTRA), a long-acting muscarinic antagonist (LAMA), theophylline, and an oral corticosteroids (OCS). In some embodiments, the LABD is selected from budesonide/formoterol fumarate dehydrate (e.g., Symbicort®), fluticasone propionate and salmeterol (e.g., Advair®), arformoterol tartrate (e.g., Brovana®), formoterol fumarate (e.g., Foradil®, Performist®), and salmeterol xinafoate (e.g., Serevent®). In certain embodiments, the method of treating asthma comprises administering to a patient the anti-IL-13/IL-17 multispecific antibody described herein and further comprises administering to the patient a corticosteroid.

In one aspect of the invention, multispecific antibodies are provided, wherein the antibodies comprise a first half antibody and a second half antibody, wherein the first half-antibody comprises a first VH/VL unit that specifically binds IL-17 and the second half antibody comprises a second VH/VL unit that specifically binds IL-13. In some embodiments, the first half antibody does not bind IL-13, and wherein the second half antibody does not bind IL-17. In some embodiments, a multispecific antibody provided herein binds to IL-17AA and IL-17AF, inhibits IL-17AA- and IL-17AF-induced activity, and/or inhibits IL-13-induced activity. In some embodiments, the multispecific antibody further binds to IL-17FF. In certain embodiments, the multispecific antibody provided herein binds to IL-17AA and IL-17AF, inhibits IL-17AA- and IL-17AF-induced activity, and inhibits IL-13-induced activity. In certain particular embodiments, the multispecific antibody provided herein binds to IL-17AA, IL-17AF and IL-17FF, inhibits IL-17AA-, IL-17AF, and IL-17FF-induced activity, and inhibits IL-13-induced activity. In certain such embodiments, the anti-IL-13/IL-17AA, AF and FF bispecific antibody advantageously block activities induced by all of IL-17A and F cytokines as opposed to activities induced by IL-17A or IL-17F alone. In some embodiments, the IL-17AA-induced activity is IL-17AA-induced gene expression and/or proliferation of cells in vivo or in vitro. In some embodiments, the IL-17AF-induced activity is IL-17AF-induced gene expression and/or proliferation of cells in vivo or in vitro. In some embodiments, the IL-13-induced activity is IL-13-induced gene expression and/or proliferation of cells in vivo or in vitro. In some embodiments, a multispecific antibody provided herein binds to IL-17AA, IL-17AF, and IL-17FF. In some embodiments, a multispecific antibody provided herein further inhibits IL-17FF-induced activity. In some such embodiments, the IL-17FF-induced activity is IL-17FF-induced gene expressing and/or proliferation of cells in vivo or in vitro. In some embodiments, a multispecific antibody provided herein does not inhibit binding of IL-13 to IL-13Rα1.

In some embodiments, a multispecific antibody is provided, wherein the first VH/VL unit comprises (a) HVR-H3 comprising the amino acid sequence of SEQ ID NO: 42 or SEQ ID NO: 44, HVR-L3 comprising the amino acid sequence of SEQ ID NO: 47, and HVR-H2 comprising an amino acid sequence selected from SEQ ID NOs: 41, 43, 80, 81, and 114; or (b) HVR-H3 comprising the amino acid sequence of SEQ ID NO: 54, HVR-L3 comprising the amino acid sequence of SEQ ID NO: 57, and HVR-H2 comprising the amino acid sequence of SEQ ID NO: 52 or SEQ ID NO: 53; or (c) HVR-H3 comprising the amino acid sequence of SEQ ID NO: 63, HVR-L3 comprising the amino acid sequence of SEQ ID NO: 66, and HVR-H2 comprising the amino acid sequence of SEQ ID NO: 62.

In some embodiments, a multispecific antibody is provided, wherein the first VH/VL unit comprises (a) HVR-H1 comprising the amino acid sequence of SEQ ID NO: 40, HVR-H2 comprising an amino acid sequence selected from SEQ ID NOs: 41, 43, 80, 81, and 114, and HVR-H3 comprising the amino acid sequence of SEQ ID NO: 42 or SEQ ID NO: 44; or (b) HVR-H1 comprising the amino acid sequence of SEQ ID NO: 51, HVR-H2 comprising the amino acid sequence of SEQ ID NO: 52 or SEQ ID NO: 53, and HVR-H3 comprising the amino acid sequence of SEQ ID NO: 54; or (c) HVR-H1 comprising the amino acid sequence of SEQ ID NO: 61, HVR-H2 comprising the amino acid sequence of SEQ ID NO: 62, and HVR-H3 comprising the amino acid sequence of SEQ ID NO: 63.

In some embodiments, a multispecific antibody is provided, wherein the first VH/VL unit comprises (a) HVR-L1 comprising the amino acid sequence of SEQ ID NO: 45, HVR-L2 comprising the amino acid sequence of SEQ ID NO: 46, and HVR-L3 comprising the amino acid sequence of SEQ ID NO: 47; or (b) HVR-L1 comprising the amino acid sequence of SEQ ID NO: 55, HVR-L2 comprising the amino acid sequence of SEQ ID NO: 56, and HVR-L3 comprising the amino acid sequence of SEQ ID NO: 57; or (c) HVR-L1 comprising the amino acid sequence of SEQ ID NO: 64, HVR-L2 comprising the amino acid sequence of SEQ ID NO: 65, and HVR-L3 comprising the amino acid sequence of SEQ ID NO: 66.

In some embodiments, a multispecific antibody is provided, wherein the first VH/VL unit comprises (a) HVR-H1 comprising the amino acid sequence of SEQ ID NO: 40, HVR-H2 comprising an amino acid sequence selected from SEQ ID NOs: 41, 43, 80, 81, and 114, HVR-H3 comprising the amino acid sequence of SEQ ID NO: 42 or SEQ ID NO: 44, HVR-L1 comprising the amino acid sequence of SEQ ID NO: 45, HVR-L2 comprising the amino acid sequence of SEQ ID NO: 46, and HVR-L3 comprising the amino acid sequence of SEQ ID NO: 47; or (b) HVR-H1 comprising the amino acid sequence of SEQ ID NO: 51, HVR-H2 comprising the amino acid sequence of SEQ ID NO: 52 or SEQ ID NO: 53, HVR-H3 comprising the amino acid sequence of SEQ ID NO: 54, HVR-L1 comprising the amino acid sequence of SEQ ID NO: 55, HVR-L2 comprising the amino acid sequence of SEQ ID NO: 56, and HVR-L3 comprising the amino acid sequence of SEQ ID NO: 57; or (c) HVR-H1 comprising the amino acid sequence of SEQ ID NO: 61, HVR-H2 comprising the amino acid sequence of SEQ ID NO: 62, HVR-H3 comprising the amino acid sequence of SEQ ID NO: 63, HVR-L1 comprising the amino acid sequence of SEQ ID NO: 64, HVR-L2 comprising the amino acid sequence of SEQ ID NO: 65, and HVR-L3 comprising the amino acid sequence of SEQ ID NO: 66.

In some embodiments, a multispecific antibody is provided, comprising a first half antibody and a second half antibody, wherein the first half-antibody comprises a first VH/VL unit that specifically binds IL-17 and the second half antibody comprises a second VH/VL unit that specifically binds IL-13, wherein the first VH/VL unit comprises HVR-H1 comprising the amino acid sequence of SEQ ID NO: 40, HVR-H2 comprising an amino acid sequence selected from SEQ ID NOs: 41, 43, 80, 81, and 114, HVR-H3 comprising the amino acid sequence of SEQ ID NO: 42 or SEQ ID NO: 44, HVR-L1 comprising the amino acid sequence of SEQ ID NO: 45, HVR-L2 comprising the amino acid sequence of SEQ ID NO: 46, and HVR-L3 comprising the amino acid sequence of SEQ ID NO: 47. In some embodiments, the first half antibody does not bind IL-13, and the second half antibody does not bind IL-17. In some embodiments, the anti-IL-13/IL-17 multispecific antibody is an anti-IL-13/IL-17 bispecific antibody that is a monovalent binder to IL-13 and a monovalent binder to IL-17. In certain embodiments, the anti-IL-13/IL-17 bispecific antibody is a monovalent binder to IL-13 and a monovalent binder to IL-17AA, AF, and FF.

In some embodiments, a multispecific antibody is provided, wherein the first VH/VL unit comprises (a) (i) a VH sequence having at least 95%, at least 96%, at least 97%, at least 98%, or at least 99% sequence identity to an amino acid sequence selected from SEQ ID NOs: 37, 39, 82, 83, and 115; or (ii) a VL sequence having at least 95%, at least 96%, at least 97%, at least 98%, or at least 99% sequence identity to the amino acid sequence of SEQ ID NO: 38; or (iii) a VH sequence as in (i) and a VL sequence as in (ii); or (b) (i) a VH sequence having at least 95%, at least 96%, at least 97%, at least 98%, or at least 99% sequence identity to the amino acid sequence or SEQ ID NO: 48 or 50; or (ii) a VL sequence having at least 95%, at least 96%, at least 97%, at least 98%, or at least 99% sequence identity to the amino acid sequence of SEQ ID NO: 49; or (iii) a VH sequence as in (i) and a VL sequence as in (ii); or (c) (i) a VH sequence having at least 95%, at least 96%, at least 97%, at least 98%, or at least 99% sequence identity to the amino acid sequence of SEQ ID NO: 58; or (ii) a VL sequence having at least 95%, at least 96%, at least 97%, at least 98%, or at least 99% sequence identity to the amino acid sequence of SEQ ID NO: 59 or 60; or (iii) a VH sequence as in (i) and a VL sequence as in (ii).

In some embodiments, the multispecific antibody comprises a first VH/VL unit comprising a VH sequence having at least 95%, at least 96%, at least 97%, at least 98%, or at least 99% sequence identity to the sequence selected from SEQ ID NOs: 37, 39, 48, 50, 58, 82, 83, and 115. In some embodiments, the first VH/VL unit comprises a VL sequence having at least 95%, at least 96%, at least 97%, at least 98%, or at least 99% sequence identity to the sequence selected from SEQ ID NOs: 38, 49, 59, and 60. In some embodiments, the first VH/VL unit comprises (a) a VH sequence selected from SEQ ID NOs: 37, 39, 82, 83, and 115 and the VL sequence of SEQ ID NO: 38; or (b) the VH sequence of SEQ ID NO: 48 or 50 and the VL sequence of SEQ ID NO: 49; (c) the VH sequence of SEQ ID NO: 58 and the VL sequence of SEQ ID NO: 59 or 60; or (d) the VH sequence of SEQ ID NO: 39 and the VL sequence of SEQ ID NO: 38.

In some embodiments, a multispecific antibody is provided, wherein the second VH/VL unit comprises (a) HVR-H3 comprising the amino acid sequence of SEQ ID NO: 17, HVR-L3 comprising the amino acid sequence of SEQ ID NO: 20, and HVR-H2 comprising the amino acid sequence of SEQ ID NO: 16; or (b) HVR-H3 comprising the amino acid sequence of SEQ ID NO: 33, HVR-L3 comprising the amino acid sequence of SEQ ID NO: 36, and HVR-H2 comprising the amino acid sequence of SEQ ID NO: 32. In some embodiments, a multispecific antibody is provided, wherein the second VH/VL unit comprises (a) HVR-H1 comprising the amino acid sequence of SEQ ID NO: 15, HVR-H2 comprising the amino acid sequence of SEQ ID NO: 16, and HVR-H3 comprising the amino acid sequence of SEQ ID NO: 17; or (b) HVR-H1 comprising the amino acid sequence of SEQ ID NO: 31, HVR-H2 comprising the amino acid sequence of SEQ ID NO: 32, and HVR-H3 comprising the amino acid sequence of SEQ ID NO: 33. In some embodiments, a multispecific antibody is provided, wherein the second VH/VL unit comprises (a) HVR-L1 comprising the amino acid sequence of SEQ ID NO: 18, HVR-L2 comprising the amino acid sequence of SEQ ID NO: 19, and HVR-L3 comprising the amino acid sequence of SEQ ID NO: 20; or (b) HVR-L1 comprising the amino acid sequence of SEQ ID NO: 34, HVR-L2 comprising the amino acid sequence of SEQ ID NO: 35, and HVR-L3 comprising the amino acid sequence of SEQ ID NO: 36. In some embodiments, a multispecific antibody is provided, wherein the second VH/VL unit comprises (a) HVR-H1 comprising the amino acid sequence of SEQ ID NO: 15, HVR-H2 comprising the amino acid sequence of SEQ ID NO: 16, HVR-H3 comprising the amino acid sequence of SEQ ID NO: 17, HVR-L1 comprising the amino acid sequence of SEQ ID NO: 18, HVR-L2 comprising the amino acid sequence of SEQ ID NO: 19, and HVR-L3 comprising the amino acid sequence of SEQ ID NO: 20; or (b) HVR-H1 comprising the amino acid sequence of SEQ ID NO: 31, HVR-H2 comprising the amino acid sequence of SEQ ID NO: 32, HVR-H3 comprising the amino acid sequence of SEQ ID NO: 33, HVR-L1 comprising the amino acid sequence of SEQ ID NO: 34, HVR-L2 comprising the amino acid sequence of SEQ ID NO: 35, and HVR-L3 comprising the amino acid sequence of SEQ ID NO: 36.

In some embodiments, a multispecific antibody is provided, wherein the multispecific antibody comprises a first half antibody and a second half antibody, wherein the first half-antibody comprises a first VH/VL unit that specifically binds IL-17 and the second half antibody comprises a second VH/VL unit that specifically binds IL-13, wherein the second VH/VL unit comprises HVR-H1 comprising the amino acid sequence of SEQ ID NO: 15, HVR-H2 comprising the amino acid sequence of SEQ ID NO: 16, HVR-H3 comprising the amino acid sequence of SEQ ID NO: 17, HVR-L1 comprising the amino acid sequence of SEQ ID NO: 18, HVR-L2 comprising the amino acid sequence of SEQ ID NO: 19, and HVR-L3 comprising the amino acid sequence of SEQ ID NO: 20. In some embodiments, the first half antibody does not bind IL-13, and the second half antibody does not bind IL-17.

In some embodiments, a multispecific antibody is provided, wherein the second VH/VL unit comprises (a) (i) a VH sequence having at least 95% sequence identity to the amino acid sequence of SEQ ID NO: 11 or 13; or (ii) a VL sequence having at least 95%, at least 96%, at least 97%, at least 98%, or at least 99% sequence identity to the amino acid sequence of SEQ ID NO: 12 or 14; or a VH sequence as in (i) and a VL sequence as in (ii); or (b) (i) a VH sequence having at least 95%, at least 96%, at least 97%, at least 98%, or at least 99% sequence identity to the amino acid sequence of SEQ ID NO: 30; (ii) a VL sequence having at least 95%, at least 96%, at least 97%, at least 98%, or at least 99% sequence identity to the amino acid sequence of SEQ ID NO: 29; or a VH sequence as in (i) and a VL sequence as in (ii). In some embodiments, the second VH/VL unit comprises the VH sequence of SEQ ID NO: 11, 13, or 30. In some embodiments, the second VH/VL unit comprises the VL sequence of SEQ ID NO: 12, 14, or 29. In some embodiments, the second VH/VL unit comprises the VH sequence of SEQ ID NO: 11 or 13 and the VL sequence of SEQ ID NO: 12 or 14; or the VH sequence of SEQ ID NO: 13 and the VL sequence of SEQ ID NO: 14; or the VH sequence of SEQ ID NO: 30 and the VL sequence of SEQ ID NO: 29.

In some embodiments, a multispecific antibody is provided, wherein the antibody competes for binding to IL-17 with an antibody comprising a VH sequence of SEQ ID NO: 39 and a VL sequence of SEQ ID NO: 38; or with an antibody comprising a VH sequence of SEQ ID NO: 50 and a VL sequence of SEQ ID NO: 49; or with an antibody comprising a VH sequence of SEQ ID NO: 58 and a VL sequence of SEQ ID NO: 60. In some embodiments, the antibody competes for binding to IL-17A homodimer and/or IL-17AF heterodimer. In some embodiments, the antibody competes for binding to IL-17F homodimer. In some embodiments, the multispecific antibody competes for binding to IL-13 with an antibody comprising a VH sequence of SEQ ID NO: 11 and a VL sequence of SEQ ID NO: 12; or with an antibody comprising a VH sequence of SEQ ID NO: 13 and a VL sequence of SEQ ID NO: 14; or with an antibody comprising a VH sequence of SEQ ID NO: 30 and a VL sequence of SEQ ID NO: 29. In some embodiments, the antibody binds an epitope within amino acids 77 to 89 of SEQ ID NO: 1, or within amino acids 82 to 89 of SEQ ID NO: 1.

In some embodiments, a multispecific antibody is provided, comprising a first half antibody and a second half antibody, wherein the first half-antibody comprises a first VH/VL unit that specifically binds IL-17 and the second half antibody comprises a second VH/VL unit that specifically binds IL-13, wherein the first VH/VL comprises HVR-H1 comprising the amino acid sequence of SEQ ID NO: 40, HVR-H2 comprising an amino acid sequence selected from SEQ ID NOs: 41, 43, 80, 81, and 114, HVR-H3 comprising the amino acid sequence of SEQ ID NO: 42 or SEQ ID NO: 44, HVR-L1 comprising the amino acid sequence of SEQ ID NO: 45, HVR-L2 comprising the amino acid sequence of SEQ ID NO: 46, and HVR-L3 comprising the amino acid sequence of SEQ ID NO: 47, and wherein the second VH/VL unit comprises HVR-H1 comprising the amino acid sequence of SEQ ID NO: 15, HVR-H2 comprising the amino acid sequence of SEQ ID NO: 16, HVR-H3 comprising the amino acid sequence of SEQ ID NO: 17, HVR-L1 comprising the amino acid sequence of SEQ ID NO: 18, HVR-L2 comprising the amino acid sequence of SEQ ID NO: 19, and HVR-L3 comprising the amino acid sequence of SEQ ID NO: 20. In some embodiments, the first half antibody does not bind IL-13, and the second half antibody does not bind IL-17.

In some embodiments, a multispecific antibody is provided, comprising a first half antibody and a second half antibody, wherein the first half-antibody comprises a first VH/VL unit that specifically binds IL-17 and the second half antibody comprises a second VH/VL unit that specifically binds IL-13, wherein the first VH/VL comprises HVR-H1 comprising the amino acid sequence of SEQ ID NO: 40, HVR-H2 comprising an amino acid sequence selected from SEQ ID NOs: 43, HVR-H3 comprising the amino acid sequence of SEQ ID NO: 44, HVR-L1 comprising the amino acid sequence of SEQ ID NO: 45, HVR-L2 comprising the amino acid sequence of SEQ ID NO: 46, and HVR-L3 comprising the amino acid sequence of SEQ ID NO: 47, and wherein the second VH/VL unit comprises HVR-H1 comprising the amino acid sequence of SEQ ID NO: 15, HVR-H2 comprising the amino acid sequence of SEQ ID NO: 16, HVR-H3 comprising the amino acid sequence of SEQ ID NO: 17, HVR-L1 comprising the amino acid sequence of SEQ ID NO: 18, HVR-L2 comprising the amino acid sequence of SEQ ID NO: 19, and HVR-L3 comprising the amino acid sequence of SEQ ID NO: 20. In some embodiments, the first half antibody does not bind IL-13, and the second half antibody does not bind IL-17.

In some embodiments, an anti-IL13/IL-17 bispecific antibody is provided, comprising a first half-antibody and a second half-antibody, wherein the first half-antibody comprises a first VH/VL unit and the second half-antibody comprises a second VH/VL unit, wherein the first VH/VL unit comprises HVR-H1 comprising the amino acid sequence of SEQ ID NO: 40, HVR-H2 comprising an amino acid sequence selected from SEQ ID NOs: 43, HVR-H3 comprising the amino acid sequence of SEQ ID NO: 44, HVR-L1 comprising the amino acid sequence of SEQ ID NO: 45, HVR-L2 comprising the amino acid sequence of SEQ ID NO: 46, and HVR-L3 comprising the amino acid sequence of SEQ ID NO: 47, and wherein the second VH/VL unit comprises HVR-H1 comprising the amino acid sequence of SEQ ID NO: 15, HVR-H2 comprising the amino acid sequence of SEQ ID NO: 16, HVR-H3 comprising the amino acid sequence of SEQ ID NO: 17, HVR-L1 comprising the amino acid sequence of SEQ ID NO: 18, HVR-L2 comprising the amino acid sequence of SEQ ID NO: 19, and HVR-L3 comprising the amino acid sequence of SEQ ID NO: 20. In certain embodiments, the anti-IL-13/IL-17 bispecific antibody comprises a second VH/VL unit that comprises the CDRs of lebrikizumab. In some embodiments, the anti-IL-13/IL-17 bispecific antibody improves the efficacy of lebrikizumab for treating an individual with moderate to severe asthma. In some embodiments, the individual has elevated level of total blood or serum periostin and/or elevated blood eosinophil counts and/or elevated neutrophil counts as compared to a control level. In some embodiments, the individual has elevated level of FE_{NO} as compared to a control level. In certain embodiments, the control level is the medium level of the same patient population. In some embodiments, the anti-IL-13/IL-17 bispecific antibody improves the efficacy of lebrikizumab for treating periostin-high, moderate to severe asthma. In some embodiments, the asthma is uncontrolled on corticosteroid. In some embodiments, the corticosteroid is inhaled corticosteroid. In some embodiments, the first half antibody does not bind IL-13, and the second half antibody does not bind IL-17.

In some embodiments, a multispecific antibody is provided, comprising a first half antibody and a second half antibody, wherein the first half-antibody comprises a first VH/VL unit that specifically binds IL-17 and the second half antibody comprises a second VH/VL unit that specifically binds IL-13, wherein the first VH/VL unit comprises the VH sequence of SEQ ID NO: 39 and the VL sequence of SEQ ID NO: 38, and the second VH/VL unit comprises the VH sequence of SEQ ID NO: 13 and the VL sequence of SEQ ID NO: 14. In some embodiments, the first half antibody does not bind IL-13, and the second half antibody does not bind IL-17.

In some embodiments, an anti-IL13/IL-17 bispecific antibody is provided, comprising a first half antibody and a second half antibody, wherein the first half-antibody comprises a first VH/VL unit and the second half-antibody comprises a second VH/VL unit, wherein the first VH/VL unit comprises the VH sequence of SEQ ID NO: 39 and the VL sequence of SEQ ID NO: 38, and the second VH/VL unit comprises the VH sequence of SEQ ID NO: 13 and the VL sequence of SEQ ID NO: 14. In some embodiments, the first half antibody does not bind IL-13, and the second half antibody does not bind IL-17.

In any of the embodiments described herein, the multispecific antibody may be an IgG antibody. In any of the embodiments described herein, the multispecific antibody may be an IgG1 or IgG4 antibody. In any of the embodiments described herein, the multispecific antibody may be an IgG4 antibody.

In any of the embodiments described herein, the multispecific antibody may comprise a first heavy chain constant region and a second heavy chain constant region, wherein the first heavy chain constant region comprises a knob mutation and the second heavy chain constant region comprises a hole mutation. In some embodiments, the first heavy chain constant region is fused to the heavy chain variable region portion of a VH/VL unit that binds IL-17. In some embodiments, the second heavy chain constant region is fused to the heavy chain variable region portion of a VH/VL unit that binds IL-13. In some embodiments, the first heavy chain constant region is fused to the heavy chain variable region portion of a VH/VL unit that binds IL-13. In some embodiments, the second heavy chain constant region is fused to the heavy chain variable region portion of a VH/VL unit that binds IL-17. In some embodiments, the antibody is an IgG1 antibody and wherein the knob mutation comprises a T366W mutation. In some embodiments, the antibody is an IgG1 antibody and wherein the hole mutation comprises at least one, at least two, or three mutations selected from T366S, L368A, and Y407V. In some embodiments, the antibody is an IgG4 antibody and wherein the knob mutation comprises a T366W mutation. In some embodiments, the antibody is an IgG4 antibody and wherein the hole mutation comprises at least one, at least two, or three mutations selected from T366S, L368A, and Y407V mutations. In some embodiments, the antibody comprises a first heavy chain constant region comprising the sequence of SEQ ID NO: 67 and a second heavy chain constant region comprising the sequence of SEQ ID NO: 68. In some embodiments, the antibody comprises a first heavy chain constant region comprising the sequence of SEQ ID NO: 69 and a second heavy chain constant region comprising the sequence of SEQ ID NO: 70.

In some embodiments, a multispecific antibody is provided, comprising a first half antibody and a second half antibody, wherein the first half-antibody specifically binds IL-17 and the second half antibody specifically binds IL-13, wherein the antibody comprises a first heavy chain comprising the sequence of SEQ ID NO: 72 or SEQ ID NO:117, a first light chain comprising the sequence of SEQ ID NO: 73, a second heavy chain comprising the sequence of SEQ ID NO: 21 or SEQ ID NO:116, and a second light chain comprising the sequence of SEQ ID NO: 22. In some embodiments, the first half antibody does not bind IL-13, and the second half antibody does not bind IL-17.

VH and the heavy chain may include an N-terminal glutamine and the heavy chain may also include a C-terminal lysine. As is known in the art, N-terminal glutamine residues can form pyroglutamate and C-terminal lysine residues can be clipped during manufacturing processes. Thus, in certain embodiments, the N-terminal glutamine may be optionally removed. In addition, heavy chains with or without the C-terminal lysine residues are both contemplated by the current invention.

In some embodiments, an isolated nucleic acid is provided that encodes any of the multispecific antibodies or isolated antibodies described herein. In some embodiments, an isolated nucleic acid is provided that encodes a first VH/VL unit of any of the multispecific antibodies described herein. In some embodiments, an isolated nucleic acid is provided that encodes a second VH/VL unit of any of the multispecific antibodies described herein. In some embodiments, a host cell is provided that comprises the isolated nucleic acid(s). In some embodiments, the host cell is a prokaryotic cell or a eukaryotic cell. In some embodiments, the host cell is an E. coli cell or a CHO cell. In certain embodiments, the multispecific antibody comprises a first VH/VL unit and a second VH/VL unit, wherein the first VH/VL unit comprises the VH sequence of SEQ ID NO: 39 and the VL sequence of SEQ ID NO: 38, and the second VH/VL unit comprises the VH sequence of SEQ ID NO: 13 and the VL sequence of SEQ ID NO: 14. In some embodiments, a method of producing an antibody is provided comprising culturing the host cell under conditions sufficient to produce the antibody. In certain embodiments, the host cell is an E. coli cell. In some embodiments, the host cell is an E. coli cell, and the multispecific antibody is aglycosylated. In certain embodiments, the method further comprises recovering the half-antibody or multispecific antibody.

In some embodiments, an isolated nucleic acid is provided, comprising (a) a sequence that is at least 90%, at least 91%, at least 92%, at least 93%, at least 94%, at least 95%, at least 96%, at least 97%, at least 98%, at least 99%, or 100% identical to the sequence of SEQ ID NO: 107 or 103 or 118; (b) a sequence that is at least 90%, at least 91%, at least 92%, at least 93%, at least 94%, at least 95%, at least 96%, at least 97%, at least 98%, at least 99%, or 100% identical to the sequence of SEQ ID NO: 108 or 104; or (c) the sequence of (a) and the sequence of (b). In some embodiments, an isolated nucleic acid is provided, comprising (a) a sequence that is at least 90%, at least 91%, at least 92%, at least 93%, at least 94%, at least 95%, at least 96%, at least 97%, at least 98%, at least 99%, or 100% identical to the sequence of SEQ ID NO: 105 or 99 or 119; (b) a sequence that is at least 90%, at least 91%, at least 92%, at least 93%, at least 94%, at least 95%, at least 96%, at least 97%, at least 98%, at least 99%, or 100% identical to the sequence of SEQ ID NO: 106 or 100; or (c) the sequence of (a) and the sequence of (b). In some embodiments, a host cell comprising a first nucleic acid comprising a sequence that is at least 90%, at least 91%, at least 92%, at least 93%, at least 94%, at least 95%, at least 96%, at least 97%, at least 98%, at least 99%, or 100% identical to the sequence of SEQ ID NO: 107 or 103 or 108 and a second nucleic acid comprising a sequence that is at least 90%, at least 91%, at least 92%, at least 93%, at least 94%, at least 95%, at least 96%, at least 97%, at least 98%, at least 99%, or 100% identical to the sequence of SEQ ID NO: 108 or 104 is provided, wherein the first nucleic acid and the second nucleic acid are comprised on the same nucleic acid molecule or on different nucleic acid molecules. In some embodiments, a host cell comprising a first nucleic acid comprising a sequence that is at least 90%, at least 91%, at least 92%, at least 93%, at least 94%, at least 95%, at least 96%, at least 97%, at least 98%, at least 99%, or 100% identical to the sequence of SEQ ID NO: 105 or 99 or 119 and a second nucleic acid comprising a sequence that is at least 90%, at least 91%, at least 92%, at least 93%, at least 94%, at least 95%, at least 96%, at least 97%, at least 98%, at least 99%, or 100% identical to the sequence of SEQ ID NO: 106 or 100 is provided, wherein the first nucleic acid and the second nucleic acid are comprised on the same nucleic acid molecule or on different nucleic acid molecules. In some embodiments, the host cell is a prokaryotic cell or a eukaryotic cell. In some embodiments, the prokaryotic cell is an E. coli cell and wherein the eukaryotic cell is a CHO cell. In some embodiments, a method of producing a half antibody or a multispecific antibody is provided, comprising culturing the host cell under conditions sufficient to produce the half antibody or multispecific antibody. In some embodiments, the method further comprises recovering the half antibody or multispecific antibody.

In some embodiments, a method of producing a multispecific antibody is provided, comprising (i) culturing a first host cell comprising a first nucleic acid comprising a sequence that is at least 90%, at least 91%, at least 92%, at least 93%, at least 94%, at least 95%, at least 96%, at least 97%, at least 98%, at least 99%, or 100% identical to the sequence of SEQ ID NO: 105 or 99 and a second nucleic acid comprising a sequence that is at least 90%, at least 91%, at least 92%, at least 93%, at least 94%, at least 95%, at least 96%, at least 97%, at least 98%, at least 99%, or 100% identical to the sequence of SEQ ID NO: 106 or 100 to produce a first half antibody, and (ii) culturing a second host cell comprising a first nucleic acid comprising a sequence that is at least 90%, at least 91%, at least 92%, at least 93%, at least 94%, at least 95%, at least 96%, at least 97%, at least 98%, at least 99%, or 100% identical to the sequence of SEQ ID NO: 107 or 103 or 118 and a second nucleic acid comprising the sequence a sequence that is at least 90%, at least 91%, at least 92%, at least 93%, at least 94%, at least 95%, at least 96%, at least 97%, at least 98%, at least 99%, or 100% identical to the sequence of SEQ ID NO: 108 or 104, wherein the first nucleic acid and the second nucleic acid are comprised on the same nucleic acid molecule or on different nucleic acid molecules, to produce a second half antibody. In some embodiments, a method of producing a multispecific antibody is provided, comprising (i) culturing a first host cell comprising a first nucleic acid comprising a sequence of SEQ ID NO: 105, 99, 119 or 101 and a second nucleic acid comprising a sequence of SEQ ID NO: 106, 100 or 102 to produce a first half antibody, and (ii) culturing a second host cell comprising a first nucleic acid comprising the sequence of SEQ ID NO: 107 or 103 or 118 and a second nucleic acid comprising the sequence of SEQ ID NO: 108 or 104, wherein the first nucleic acid and the second nucleic acid are comprised on the same nucleic acid molecule or on different nucleic acid molecules, to produce a second half antibody. In some embodiments, a method of producing a multispecific antibody is provided, comprising (i) culturing a first host cell comprising a first nucleic acid comprising a sequence of SEQ ID NO: 105 or 99 or 119 and a second nucleic acid comprising a sequence of SEQ ID NO: 106 or 100 to produce a first half antibody, and (ii) culturing a second host cell comprising a first nucleic acid comprising the sequence of SEQ ID NO: 107 or 103 or 118 and a second nucleic acid comprising the sequence of SEQ ID NO: 108 or 104, wherein the first nucleic acid and the second nucleic acid are comprised on the same nucleic acid molecule or on different nucleic acid molecules, to produce a second half antibody. In some embodiments, the method further comprises recovering the first half antibody and recovering the second half antibody. In some embodiments, the method comprises forming a mixture comprising the first half antibody and the second half antibody under conditions sufficient to produce a multispecific antibody. In some embodiments, a multispecific antibody produced by the methods described herein is provided. In certain embodiments, the multispecific antibody comprises a first VH/VL unit and a second VH/VL unit, wherein the first VH/VL unit comprises the VH sequence of SEQ ID NO: 39 and the VL sequence of SEQ ID NO: 38, and the second VH/VL unit comprises the VH sequence of SEQ ID NO: 13 and the VL sequence of SEQ ID NO: 14. In certain embodiments, the same or different nucleic acid molecules can be one or more vectors, in particular expression vectors.

In some embodiments, an immunoconjugate is provided, wherein the immunoconjugate comprises any of the multispecific antibodies or isolated antibodies described herein and a cytotoxic agent.

In some embodiments, pharmaceutical formulations are provided, comprising any of the multispecific antibodies or isolated antibodies described herein and a pharmaceutically acceptable carrier.

### BRIEF DESCRIPTION OF THE DRAWINGS

FIG. 1A-C show pairwise comparisons of bronchial biopsy tissue counts for (A) neutrophils and eosinophils, (B) eosinophils and IL-17A+ cells, and (C) IL-17F+ and IL-17A+ cells, as described in Example 1.

FIG. 2A-B show (A) IL-17A and (B) IL-17F expression in matched biopsy neutrophil samples with respect to undetectable (less than the lower limit of quantitation; <LLOQ) and detectable (greater than or equal to the lower limit of quantitation, ≧LLOQ), as described in Example 2.

FIG. 3 shows two-way hierarchical clustering of certain neutrophil-associated gene expression from microarray analyses of UK cohort bronchial biopsies, as described in Example 2. Open circles correspond asthmatics not taking steroids. Closed circles indicate asthmatics taking steroids (inhaled corticosteroids (ICS) or oral corticosteroids (OCS)).

FIG. 4A-B show (A) CXCL1 levels in healthy controls and moderate-severe asthmatics from the BOBCAT study, and (B) plasma CXCL1 levels in asthmatic patients with serum periostin levels below 50 ng/ml or equal to or above 50 ng/ml, as described in Example 3. CXCL1 levels above 160 pg/ml (dashed line) tend to have elevated serum periostin levels (p=0.02 by Fisher's exact test).

FIG. 5 shows serum periostin levels in the UK Cohort with undetectable (<LLOQ) or detectable (≧LLOQ) IL-17A expression in matching samples, as described in Example 3. Serum periostin is elevated in subjects with detectable bronchial biopsy IL-17A mRNA (P=0.03, Kruskal-Wallis).

FIG. 6A-D show (A, C) eosinophil numbers and (B, D) neutrophil numbers in bronchoalveolar lavage (BAL; A, B) and blood (C, D) in a mouse house dust mite asthma model following administration of an anti-IL-13 antibody, a mixture of anti-IL-17AA/AF antibody and anti-IL-17FF antibody, or a mixture of all three antibodies, as described in Example 4. *p<0.05, **p<0.005.

FIG. 7A-D show (A) SDS-PAGE analysis of the anti-IL-13 knob and anti-IL-17 hole half antibodies, (B) SEC analysis of the bispecific antibody, (C) SDS PAGE analysis of the bispecific antibody under nonreducing (lane a) and reducing (lane c) conditions, and (D), LC-ESI/TOF analysis of the F(ab′)₂ fragments of the bispecific antibody, as described in Example 6.

FIG. 8A-B show dose-dependent inhibition of (A) IL-13-induced and (B) IL-13 R130Q-induced proliferation of TF-1 cells by lebrikizumab and anti-IL-13/IL-17 bispecific antibody, as described in Example 8.

FIG. 9A-C show dose-dependent inhibition of (A) IL-17A-induced and (B) IL-17AF-induced, and (C) IL-17F-induced expression of G-CSF in normal human foreskin fibroblasts by anti-IL-17 antibody and anti-IL-13/IL-17 bispecific antibody, as described in Example 8.

FIG. 10A-B show dose-dependent inhibition of both (A) IL-13-induced CCL26 expression and (B) IL-17A-induced CXCL1 expression by anti-IL-13/IL-17 bispecific antibody, as described in Example 9.

FIG. 11 shows serum concentration following a single intravenous dose of anti-IL-13/IL-17 bispecific antibody in mice, as described in Example 10.

FIG. 12 shows serum concentration in individual cynomolgus monkeys following a single intravenous dose of anti-IL-13/IL-17 bispecific antibody, as described in Example 11.

FIG. 13 shows IL-17A homodimer levels following administration of anti-IL-13/IL-17 bispecific antibody to cynomolgus monkeys, as described in Example 11.

FIG. 14A-C show (A) plasma TARC levels, (B) serum G-CSF levels, and (C) serum CXCL1 levels in house dust mite asthma model mice following administration of anti-IL-13 antibody, anti-IL-13/IL-17 bispecific antibody, or anti-IL-17 antibody, as described in Example 13.

FIG. 15A-D show graphs in which percent change in FEV1 for placebo and lebrikizumab arms of the clinical studies are plotted into four groups defined by base line eosinophil counts and neutrophil counts (A: eosinophil-low and neutrophil-high; B: eosinophil-low and neutrophil-low; C: eosinophil-high and neutrophil-high; and D: eosinophil-high and neutrophil-low) as described in Example 5. The number of subjects underlying these analyses is annotated in each respective sub-plot.

### DETAILED DESCRIPTION

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

### Certain Definitions

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

As used in this specification and the appended claims, the singular forms “a,” “an” and “the” include plural referents unless the context clearly dictates otherwise. Thus, for example, reference to “a protein” or an “antibody” includes a plurality of proteins or antibodies, respectively; reference to “a cell” includes mixtures of cells, and the like.

The term “biological sample” as used herein includes, but is not limited to, blood, serum, plasma, sputum, bronchoalveolar lavage, tissue biopsies (e.g., lung samples), and nasal samples including nasal swabs or nasal polyps.

FE_{NO} assay refers to an assay that measures FE_{NO} (fractional exhaled nitric oxide) levels. Such levels can be evaluated using, e.g., a hand-held portable device, NIOX MINO™ (Aerocrine, Solna, Sweden), in accordance with guidelines published by the American Thoracic Society (ATS) in 2005. FE_{NO} may be noted in other similar ways, e.g., FeNO or FENO, and it should be understood that all such similar variations have the same meaning.

Asthma is a complex disorder characterized by variable and recurring symptoms, reversible airflow obstruction (e.g., by bronchodilator) and bronchial hyper-responsiveness which may or may not be associated with underlying inflammation. Examples of asthma include aspirin sensitive/exacerbated asthma, atopic asthma, severe asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids and other asthmas as mentioned in J Allergy Clin Immunol (2010) 126(5):926-938.

“Eosinophilic Disorder” means a disorder associated with excess eosinophil numbers in which atypical symptoms may manifest due to the levels or activity of eosinophils locally or systemically in the body. In certain embodiments, excess blood eosinophil count is at least 200/μl, at least 250/μl, at least 300/μl, or at least 400/μl. In certain embodiments, the individual has been determined to have an elevated blood eosinophil count as compared to a control or reference level. In certain embodiments, the individual has been determined to have a baseline blood eosinophil count of at least 150/μl, at least 200/μl, at least 250/μl, at least 300/μl, or at least 400/μl. Disorders associated with excess eosinophil numbers or activity include but are not limited to, asthma (including aspirin sensitive asthma), atopic asthma, atopic dermatitis, allergic rhinitis (including seasonal allergic rhinitis), non-allergic rhinitis, asthma, severe asthma, chronic eosinophilic pneumonia, allergic bronchopulmonary aspergillosis, coeliac disease, Churg-Strauss syndrome (periarteritis nodosa plus atopy), eosinophilic myalgia syndrome, hypereosinophilic syndrome, oedematous reactions including episodic angiodema, helminth infections, where eosinophils may have a protective role, onchocercal dermatitis and Eosinophil-Associated Gastrointestinal Disorders, including but not limited to, eosinophilic esophagitis, eosinophilic gastritis, eosinophilic gastroenteritis, eosinophilic enteritis and eosinophilic colitis, nasal micropolyposis and polyposis, aspirin intolerance, asthma and obstructive sleep apnoea. Eosinophil-derived secretory products have also been associated with the promotion of angiogenesis and connective tissue formation in tumors and the fibrotic responses seen in conditions such as chronic asthma, Crohn's disease, scleroderma and endomyocardial fibrosis (Munitz A, Levi-Schaffer F. Allergy 2004; 59: 268-75, Adamko et al. Allergy 2005; 60: 13-22, Oldhoff, et al. Allergy 2005; 60: 693-6). Other examples include cancer (e.g., glioblastoma (such as glioblastoma multiforme), non-Hodgkin's lymphoma (NHL)), atopic dermatitis, allergic rhinitis, asthma, fibrosis, inflammatory bowel disease, pulmonary fibrosis (including idiopathic pulmonary fibrosis (IPF) and pulmonary fibrosis secondary to sclerosis), COPD, hepatic fibrosis. In certain embodiments, the patient is identified as an Eosinophilic Inflammation Positive (EIP) patient that shows elevated periostin levels and/or elevated levels of one or more selected from CSF1, MEIS2, LGALS12, IDO1, THBS4, OLIG2, ALOX15, SIGLEC8, CCL23, PYROXD2, HSD3B7, SORD, ASB2, CACNG6, GPR44, MGAT3, SLC47A1, SMPD3, CCR3, CLC, CYP4F12, and ABTB2, as compared to a control.

“Neutrophilic Disorder” means a disorder associated with excess neutrophil numbers. In some embodiments, atypical symptoms may manifest in a neutrophilic disorder due to the levels or activity of neutrophils locally or systemically in the body. In certain embodiments, excess blood neutrophil count is at least 3000/μl, 3500/μl, 4000/μl, or 4500/μl. In certain embodiments, the individual has been determined to have an elevated blood neutrophil count as compared to a control or reference level. In certain embodiments, the individual has been determined to have a baseline blood neutrophil count of at least 3000/μl, 3500/μl, 4000/μl, or 4500/μl. Disorders associated with excess neutrophil numbers or activity include, but are not limited to atopic dermatitis, allergic rhinitis, asthma, fibrosis, inflammatory bowel disease, Crohn's disease, lung inflammatory disorders, pulmonary fibrosis, idiopathic pulmonary fibrosis (IPF), chronic obstructive pulmonary disease (COPD), hepatic fibrosis, a respiratory disorder, cancer, glioblastoma, and non-Hodgkin's lymphoma. In any of the embodiments described herein, the respiratory disorder may be selected from asthma, allergic asthma, non-allergic asthma, bronchitis, chronic bronchitis, chronic obstructive pulmonary disease (COPD), emphysema, cigarette-induced emphysema, airway inflammation, cystic fibrosis, pulmonary fibrosis, allergic rhinitis, and bronchiectasis. In some embodiments, the individual has been determined to have a baseline blood neutrophil count that is above the medium baseline blood neutrophil count in a patient population. In some embodiments, individual with elevated neutrophil counts has a baseline blood neutrophil count that is above the medium baseline blood neutrophil count in a patient population of moderate to severe asthma.

IL-13 mediated disorder means a disorder associated with excess IL-13 levels or activity in which atypical symptoms may manifest due to the levels or activity of IL-13 locally and/or systemically in the body. Examples of IL-13 mediated disorders include cancers (e.g., non-Hodgkin's lymphoma, glioblastoma), atopic dermatitis, allergic rhinitis, asthma, fibrosis, inflammatory bowel disease, Crohn's disease, lung inflammatory disorders (including pulmonary fibrosis such as IPF), COPD, and hepatic fibrosis.

IL-17 mediated disorder means a disorder associated with excess IL-17 levels or activity in which atypical symptoms may manifest due to the levels or activity of IL-17 locally and/or systemically in the body. Examples of IL-17 mediated disorders include: atopic dermatitis, allergic rhinitis, asthma, fibrosis, inflammatory bowel disease, Crohn's disease, lung inflammatory disorders, pulmonary fibrosis, idiopathic pulmonary fibrosis (IPF), chronic obstructive pulmonary disease (COPD), hepatic fibrosis, a respiratory disorder, cancer, glioblastoma, and non-Hodgkin's lymphoma. In any of the embodiments described herein, the respiratory disorder may be selected from asthma, allergic asthma, non-allergic asthma, bronchitis, chronic bronchitis, chronic obstructive pulmonary disease (COPD), emphysema, cigarette-induced emphysema, airway inflammation, cystic fibrosis, pulmonary fibrosis, allergic rhinitis, and bronchiectasis.

Asthma-Like Symptom includes a symptom selected from the group consisting of shortness of breath, cough (changes in sputum production and/or sputum quality and/or cough frequency), wheezing, chest tightness, bronchioconstriction and nocturnal awakenings ascribed to one of the symptoms above or a combination of these symptoms (Juniper et al (2000) Am. J. Respir. Crit. Care Med., 162(4), 1330-1334.).

The term “respiratory disorder” includes, but is not limited to, asthma (e.g., allergic and non-allergic asthma (e.g., due to infection, e.g., with respiratory syncytial virus (RSV), e.g., in younger children)); bronchitis (e.g., chronic bronchitis); chronic obstructive pulmonary disease (COPD) (e.g., emphysema (e.g., cigarette-induced emphysema)); conditions involving airway inflammation, eosinophilia, fibrosis and excess mucus production, e.g., cystic fibrosis, pulmonary fibrosis, and allergic rhinitis. Examples of diseases that can be characterized by airway inflammation, excessive airway secretion, and airway obstruction include asthma, chronic bronchitis, bronchiectasis, and cystic fibrosis.

Exacerbations (commonly referred to as asthma attacks or acute asthma) are episodes of new or progressive increase in shortness of breath, cough (changes in sputum production and/or sputum quality and/or cough frequency), wheezing, chest tightness, nocturnal awakenings ascribed to one of the symptoms above or a combination of these symptoms. Exacerbations are often characterized by decreases in expiratory airflow (PEF or FEV1). However, PEF variability does not usually increase during an exacerbation, although it may do so leading up to or during the recovery from an exacerbation. The severity of exacerbations ranges from mild to life-threatening and can be evaluated based on both symptoms and lung function. Severe asthma exacerbations as described herein include exacerbations that result in any one or combination of the following hospitalization for asthma treatment, high corticosteroid use (e.g., quadrupling the total daily corticosteroid dose or a total daily dose of greater or equal to 500 micrograms of fluticasone propionate (FP) or equivalent for three consecutive days or more), or oral/parenteral corticosteroid use.

A “TH2 pathway inhibitor” or “TH2 inhibitor” is an agent that inhibits the TH2 pathway. Examples of a TH2 pathway inhibitor include inhibitors of the activity of any one of the targets selected from ITK, BTK, IL-9 (e.g., MEDI-528), IL-5 (e.g., Mepolizumab, CAS No. 196078-29-2; resilizumab), IL-13 (e.g., IMA-026, IMA-638 (also referred to as, anrukinzumab, INN No. 910649-32-0; QAX-576; IL-4/IL-13 trap), tralokinumab (also referred to as CAT-354, CAS No. 1044515-88-9); AER-001, ABT-308 (also referred to as humanized 13C5.5 antibody), IL-4 (e.g., AER-001, IL-4/IL-13 trap), OX40L, TSLP, IL-25, IL-33 and IgE (e.g., XOLAIR, QGE-031; MEDI-4212); and receptors such as: IL-9 receptor, IL-5 receptor (e.g., MEDI-563 (benralizumab, CAS No. 1044511-01-4), IL-4 receptor alpha (e.g., AMG-317, AIR-645), IL-13 receptoralpha1 (e.g., R-1671) and IL-13 receptoralpha2, OX40, TSLP-R, IL-7Ralpha (a co-receptor for TSLP), IL-17RB (receptor for IL-25), ST2 (receptor for IL-33), CCR3, CCR4, CRTH2 (e.g., AMG-853, AP768, AP-761, MLN6095, ACT129968), FcepsilonRI, FcepsilonRII/CD23 (receptors for IgE), Flap (e.g., GSK2190915), Syk kinase (R-343, PF3526299); CCR4 (AMG-761), TLR9 (QAX-935) and multi-cytokine inhibitor of CCR3, IL5, IL3, GM-CSF (e.g., TPI ASM8). Examples of inhibitors of the aforementioned targets are disclosed in, for example, WO2008/086395; WO2006/085938; U.S. Pat. No. 7,615,213; U.S. Pat. No. 7,501,121; WO2006/085938; WO 2007/080174; U.S. Pat. No. 7,807,788; WO2005007699; WO2007036745; WO2009/009775; WO2007/082068; WO2010/073119; WO2007/045477; WO2008/134724; US2009/0047277; and WO2008/127271.

A “TH17 pathway inhibitor” or “TH17 inhibitor” is an agent that inhibits the TH17 pathway. Examples of a TH17 pathway inhibitor include inhibitors of the activity of any one of the targets selected from IL-1β, IL-6 (e.g., tocilizumab), IL-17A (e.g., secukinumab, ixekizumab, ABT-122), IL-17F, IL-17AF heterodimer, IL-17B, IL-17C, IL-17D, IL-22, IL-21, TGF-β, IL-23, IL-1β receptor, IL-6 receptor, IL-17RA (e.g., brodalumab), IL-17RC, IL-17RB, IL-22R1, IL10R2, IL-21 receptor, TGF-β receptor, and IL-23 receptor (IL-12Rb1, IL23R). Examples of inhibitors of the aforementioned targets are disclosed in, for example, U.S. Pat. No. 7,807,15; U.S. Pat. No. 7,838,638; U.S. Pat. No. 8,580,265; US 20140314743; U.S. Pat. No. 8,519,107; U.S. Pat. No. 7,833,527; WO2014044758; US 20130005659; and WO2011023685.

The term “small molecule” refers to an organic molecule having a molecular weight between 50 Daltons to 2500 Daltons.

The term “antibody” is used in the broadest sense and specifically covers, for example, monoclonal antibodies, polyclonal antibodies, antibodies with polyepitopic specificity, single chain antibodies, multi-specific antibodies and fragments of antibodies, including antigen-binding fragments. Such antibodies can be chimeric, humanized, human and synthetic. Such antibodies and methods of generating them are described in more detail below.

The term “half-antibody” or “hemimer” as used herein refers to a monovalent antigen binding polypeptide. In certain embodiments, a half antibody or hemimer comprises a VH/VL unit and optionally at least a portion of an immunoglobulin constant domain. In certain embodiments, a half antibody or hemimer comprises one immunoglobulin heavy chain associated with one immunoglobulin light chain, or an antigen binding fragment thereof. In certain embodiments, a half antibody or hemimer is mono-specific, i.e., binds to a single antigen or epitope. In certain such embodiments, a half antibody binds to IL-13 and does not bind to IL-17. In certain other embodiments, a half antibody binds to IL-17 and does not bind to IL-13. One skilled in the art will readily appreciate that a half-antibody may have an antigen binding domain consisting of a single variable domain, e.g., originating from a camelidae.

The term “VH/VL unit” refers to the antigen-binding region of an antibody that comprises at least one VH HVR and at least one VL HVR. In certain embodiments, the VH/VL unit comprises at least one, at least two, or all three VH HVRs and at least one, at least two, or all three VL HVRs. In certain embodiments, the VH/VL unit further comprises at least a portion of a framework region (FR). In some embodiments, a VH/VL unit comprises three VH HVRs and three VL HVRs. In some such embodiments, a VH/VL unit comprises at least one, at least two, at least three or all four VH FRs and at least one, at least two, at least three or all four VL FRs.

The term “multispecific antibody” is used in the broadest sense and specifically covers an antibody comprising an antigen-binding domain that has polyepitopic specificity (i.e., is capable of specifically binding to two, or more, different epitopes on one biological molecule or is capable of specifically binding to epitopes on two, or more, different biological molecules). In some embodiments, an antigen-binding domain of a multispecific antibody (such as a bispecific antibody) comprises two VH/VL units, wherein a first VH/VL unit specifically binds to a first epitope and a second VH/VL unit specifically binds to a second epitope, wherein each VH/VL unit comprises a heavy chain variable domain (VH) and a light chain variable domain (VL). Such multispecific antibodies include, but are not limited to, full length antibodies, antibodies having two or more VL and VH domains, antibody fragments such as Fab, Fv, dsFv, scFv, diabodies, bispecific diabodies and triabodies, antibody fragments that have been linked covalently or non-covalently. A VH/VL unit that further comprises at least a portion of a heavy chain constant region and/or at least a portion of a light chain constant region may also be referred to as a “hemimer” or “half antibody.” In some embodiments, a half antibody comprises at least a portion of a single heavy chain variable region and at least a portion of a single light chain variable region. In some such embodiments, a bispecific antibody that comprises two half antibodies and binds to two antigens comprises a first half antibody that binds to the first antigen or first epitope but not to the second antigen or second epitope and a second half antibody that binds to the second antigen or second epitope and not to the first antigen or first epitope. According to some embodiments, the multispecific antibody is an IgG antibody that binds to each antigen or epitope with an affinity of 5 μM to 0.001 pM, 3 μM to 0.001 pM, 1 μM to 0.001 pM, 0.5 μM to 0.001 pM, or 0.1 μM to 0.001 pM. In some embodiments, a hemimer comprises a sufficient portion of a heavy chain variable region to allow intramolecular disulfide bonds to be formed with a second hemimer. In some embodiments, a hemimer comprises a knob mutation or a hole mutation, for example, to allow heterodimerization with a second hemimer or half antibody that comprises a complementary hole mutation or knob mutation. Knob mutations and hole mutations are discussed further below.

A “bispecific antibody” is a multispecific antibody comprising an antigen-binding domain that is capable of specifically binding to two different epitopes on one biological molecule or is capable of specifically binding to epitopes on two different biological molecules. A bispecific antibody may also be referred to herein as having “dual specificity” or as being “dual specific.” Unless otherwise indicated, the order in which the antigens bound by a bispecific antibody are listed in a bispecific antibody name is arbitrary. That is, in some embodiments, the terms “anti-IL-13/IL-17 bispecific antibody” and “anti-IL-17/IL-13 bispecific antibody” may be used interchangeably. In some embodiments, a bispecific antibody comprises two half antibodies, wherein each half antibody comprises a single heavy chain variable region and optionally at least a portion of a heavy chain constant region, and a single light chain variable region and optionally at least a portion of a light chain constant region. In certain embodiments, a bispecific antibody comprises two half antibodies, wherein each half antibody comprises a single heavy chain variable region and a single light chain variable region and does not comprise more than one single heavy chain variable region and does not comprise more than one single light chain variable region. In some embodiments, a bispecific antibody comprises two half antibodies, wherein each half antibody comprises a single heavy chain variable region and a single light chain variable region, and wherein the first half antibody binds to a first antigen and not to a second antigen and the second half antibody binds to the second antigen and not to the first antigen.

The term “knob-into-hole” or “KnH” technology as used herein refers to the technology directing the pairing of two polypeptides together in vitro or in vivo by introducing a protuberance (knob) into one polypeptide and a cavity (hole) into the other polypeptide at an interface in which they interact. For example, KnHs have been introduced in the Fc:Fc binding interfaces, C_{L}:C_{H1} interfaces or V_{H}/V_{L} interfaces of antibodies (see, e.g., US 2011/0287009, US2007/0178552, WO 96/027011, WO 98/050431, and Zhu et al., 1997, Protein Science 6:781-788). In some embodiments, KnHs drive the pairing of two different heavy chains together during the manufacture of multispecific antibodies. For example, multispecific antibodies having KnH in their Fc regions can further comprise single variable domains linked to each Fc region, or further comprise different heavy chain variable domains that pair with similar or different light chain variable domains. KnH technology can also be used to pair two different receptor extracellular domains together or any other polypeptide sequences that comprises different target recognition sequences (e.g., including affibodies, peptibodies and other Fc fusions).

The term “knob mutation” as used herein refers to a mutation that introduces a protuberance (knob) into a polypeptide at an interface in which the polypeptide interacts with another polypeptide. In some embodiments, the other polypeptide has a hole mutation (see e.g., U.S. Pat. No. 5,731,168, U.S. Pat. No. 5,807,706, U.S. Pat. No. 5,821,333, U.S. Pat. No. 7,695,936, U.S. Pat. No. 8,216,805, each incorporated herein by reference in its entirety).

The term “hole mutation” as used herein refers to a mutation that introduces a cavity (hole) into a polypeptide at an interface in which the polypeptide interacts with another polypeptide. In some embodiments, the other polypeptide has a knob mutation (see e.g., U.S. Pat. No. 5,731,168, U.S. Pat. No. 5,807,706, U.S. Pat. No. 5,821,333, U.S. Pat. No. 7,695,936, U.S. Pat. No. 8,216,805, each incorporated herein by reference in its entirety).

An antibody “inhibits” an activity induced by or associated with an antigen, such as an IL-17- and/or IL-13-induced activity, when the activity is reduced as compared to the activity measured in the absence of the antibody. In certain embodiments, an antibody inhibits an activity of the antigen by at least 10% in the presence of the antibody compared to the activity in the absence of the antibody. In some embodiments, an antibody inhibits an activity by at least 20%, at least 30%, at least 40%, at least 50%, at least 60%, at least 70%, at least 80%, or at least 90% or 100%. An antibody is considered to “neutralize” an antigen or its associated activity when the activity is reduced by at least 50% in the presence of the antibody compared to the activity in the absence of the antibody. In some embodiments, a neutralizing antibody inhibits the activity by at least 60%, at least 70%, at least 80%, or at least 90% or 100%. In certain embodiments, the IL-17- and/or IL-13-induced activity is proliferation of cells in vitro or in vivo. In certain other embodiments, the IL-17- and/or IL-13-induced activity is IL-17 mediated and/or IL-13 mediated inflammatory responses or immune-related disorders. In other embodiments, the IL-17- and/or IL-13-induced activity is IL-17 mediated and/or IL-13 mediated infiltration of inflammatory cells.

The term “therapeutic agent” refers to any agent that is used to treat a disease. A therapeutic agent may be, for example, a polypeptide(s) (e.g., an antibody, an immunoadhesin or a peptibody), an aptamer or a small molecule that can bind to a protein or a nucleic acid molecule that can bind to a nucleic acid molecule encoding a target (i.e., siRNA), etc.

The term “controller” or “preventor” refers to any therapeutic agent that is used to control asthma inflammation. Examples of controllers include corticosteroids, leukotriene receptor antagonists (e.g., inhibit the synthesis or activity of leukotrienes such as montelukast, zileuton, pranlukast, zafirlukast), LABAs, corticosteroid/LABA combination compositions, theophylline (including aminophylline), cromolyn sodium, nedocromil sodium, omalizumab, LAMAs, MABA (e.g, bifunctional muscarinic antagonist-beta2 Agonist), 5-Lipoxygenase Activating Protein (FLAP) inhibitors, and enzyme PDE-4 inhibitor (e.g., roflumilast). A “second controller” typically refers to a controller that is not the same as the first controller.

The term “corticosteroid sparing” or “CS” means the decrease in frequency and/or amount, or the elimination of, corticosteroid used to treat a disease in a patient taking corticosteroids for the treatment of the disease due to the administration of another therapeutic agent. A “CS agent” refers to a therapeutic agent that can cause CS in a patient taking a corticosteroid.

The term “corticosteroid” includes, but is not limited to fluticasone (including fluticasone propionate (FP)), beclometasone, budesonide, ciclesonide, mometasone, flunisolide, betamethasone and triamcinolone. “Inhalable corticosteroid” means a corticosteroid that is suitable for delivery by inhalation. Exemplary inhalable corticosteroids are fluticasone, beclomethasone dipropionate, budenoside, mometasone furoate, ciclesonide, flunisolide, triamcinolone acetonide and any other corticosteroid currently available or becoming available in the future. Examples of corticosteroids that can be inhaled and are combined with a long-acting beta2-agonist include, but are not limited to: budesonide/formoterol and fluticasone/salmeterol.

Examples of corticosteroid/LABA combination drugs include fluticasone furoate/vilanterol trifenatate and indacaterol/mometasone.

The term “LABA” means long-acting beta-2 agonist, which agonist includes, for example, salmeterol, formoterol, bambuterol, albuterol, indacaterol, arformoterol and clenbuterol.

The term “LAMA” means long-acting muscarinic antagonist, which agonists include: tiotropium.

Examples of LABA/LAMA combinations include, but are not limited to: olodaterol tiotropium (Boehringer Ingelheim's) and indacaterol glycopyrronium (Novartis)

The term “SABA” means short-acting beta-2 agonists, which agonists include, but are not limited to, salbutamol, levosalbutamol, fenoterol, terbutaline, pirbuterol, procaterol, bitolterol, rimiterol, carbuterol, tulobuterol and reproterol

Leukotriene receptor antagonists (sometimes referred to as a leukast) (LTRA) are drugs that inhibit leukotrienes. Examples of leukotriene inhibitors include montelukast, zileuton, pranlukast, and zafirlukast.

The term “FEV1” refers to the volume of air exhaled in the first second of a forced expiration. It is a measure of airway obstruction. Provocative concentration of methacholine required to induce a 20% decline in FEV1 (PC20) is a measure of airway hyper-responsiveness. FEV1 may be noted in other similar ways, e.g., FEV₁, and it should be understood that all such similar variations have the same meaning.

The term “relative change in FEV1”=(FEV1 at week 12 of treatment−FEV1 prior to start of treatment) divided by FEV1.

The term “PEF” means peak expiratory flow, which refers to the maximal flow achieved during the maximally forced expiration after full inspiration. It is a parameter that can be used to measure airway function.

As used herein, “FVC” refers to “Forced Vital Capacity” which refers to a standard test that measures the change in lung air volume between a full inspiration and maximal expiration to residual volume (as opposed to the volume of air expelled in one second as in FEV1). It is a measure of the functional lung capacity. In patients with restrictive lung diseases such as interstitial lung disease including IPF, hypersensitivity pneumonitis, sarcoidosis, and systemic sclerosis, the FVC is reduced typically due to scarring of the lung parenchyma.

The term “mild asthma” refers to a patient generally experiencing symptoms or exacerbations less than two times a week, nocturnal symptoms less than two times a month, and is asymptomatic between exacerbations. Mild, intermittent asthma is often treated as needed with the following: inhaled bronchodilators (short-acting inhaled beta2-agonists); avoidance of known triggers; annual influenza vaccination; pneumococcal vaccination every 6 to 10 years, and in some cases, an inhaled beta2-agonist, cromolyn, or nedocromil prior to exposure to identified triggers. If the patient has an increasing need for short-acting beta2-agonist (e.g., uses short-acting beta2-agonist more than three to four times in 1 day for an acute exacerbation or uses more than one canister a month for symptoms), the patient may require a stepup in therapy.

The term “moderate asthma” generally refers to asthma in which the patient experiences exacerbations more than two times a week and the exacerbations affect sleep and activity; the patient has nighttime awakenings due to asthma more than two times a month; the patient has chronic asthma symptoms that require short-acting inhaled beta2-agonist daily or every other day; and the patient's pretreatment baseline PEF or FEV1 is 60 to 80 percent predicted and PEF variability is 20 to 30 percent.

The term “severe asthma” generally refers to asthma in which the patient has almost continuous symptoms, frequent exacerbations, frequent nighttime awakenings due to the asthma, limited activities, PEF or FEV1 baseline less than 60 percent predicted, and PEF variability of 20 to 30 percent.

Examples of rescue medications include albuterol, ventolin and others.

“Resistant” refers to a disease that demonstrates little or no clinically significant improvement after treatment with a therapeutic agent. For example, asthma which requires treatment with high dose ICS (e.g., quadrupling the total daily corticosteroid dose or a total daily dose of greater or equal to 500 micrograms of FP (or equivalent) for at least three consecutive days or more, or systemic corticosteroid for a two week trial to establish if asthma remains uncontrolled or FEV1 does not improve is often considered severe refractory asthma.

A therapeutic agent as provided herein can be administered by any suitable means, including parenteral, subcutaneous, intraperitoneal, intrapulmonary, and intranasal. Parenteral infusions include intramuscular, intravenous, intraarterial, intraperitoneal, or subcutaneous administration. In some embodiments, the therapeutic agent is inhaled. According to some embodiments, the dosing is given by injections, e.g., intravenous or subcutaneous injections. In some embodiments, the therapeutic agent is administered using a syringe (e.g., prefilled or not) or an autoinjector.

For the prevention or treatment of disease, the appropriate dosage of a therapeutic agent may depend on the type of disease to be treated, the severity and course of the disease, whether the therapeutic agent is administered for preventive or therapeutic purposes, previous therapy, the patient's clinical history and response to the therapeutic agent, and the discretion of the attending physician. The therapeutic agent is suitably administered to the patient at one time or over a series of treatments. The therapeutic agent composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

“Patient response” or “response” (and grammatical variations thereof) can be assessed using any endpoint indicating a benefit to the patient, including, without limitation, (1) inhibition, to some extent, of disease progression, including slowing down and complete arrest; (2) reduction in the number of disease episodes and/or symptoms; (3) reduction in lesional size; (4) inhibition (i.e., reduction, slowing down or complete stopping) of disease cell infiltration into adjacent peripheral organs and/or tissues; (5) inhibition (i.e. reduction, slowing down or complete stopping) of disease spread; (6) decrease of auto-immune response, which may, but does not have to, result in the regression or ablation of the disease lesion; (7) relief, to some extent, of one or more symptoms associated with the disorder; (8) increase in the length of disease-free presentation following treatment; and/or (9) decreased mortality at a given point of time following treatment.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described herein.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-IL-17 antibody” and “an antibody that binds to IL-17” as used herein refer to an antibody that is capable of binding IL-17A homodimer, IL-17F homodimer, and/or IL-17AF heterodimer with sufficient affinity such that the antibody is useful as a diagnostic and/or therapeutic agent in targeting IL-17. In some embodiments, the extent of binding of an anti-IL-17 antibody to an unrelated, non-IL-17 protein is less than about 10% of the binding of the antibody to IL-17 as measured, e.g., by a radioimmunoassay (RIA). In certain embodiments, an antibody that binds to IL-17 has a dissociation constant (Kd) of ≦1 μM, ≦100 nM, ≦10 nM, ≦1 nM, ≦0.1 nM, ≦0.01 nM, or ≦0.001 nM (e.g. 10-8 M or less, e.g. from 10-8 M to 10-13 M, e.g., from 10-9 M to 10-13 M). In certain embodiments, an anti-IL-17 antibody binds to an epitope of IL-17 that is conserved among IL-17 from different species. In some embodiments, an anti-IL-17 antibody is a multispecific antibody, such as a bispecific antibody.

In some embodiments, an anti-IL17 antibody is capable of binding IL-17A homodimer. In some embodiments, an anti-IL17 antibody is capable of binding IL-17A homodimer and IL-17AF heterodimer. In some embodiments, an anti-IL-17 antibody is capable of binding IL-17A homodimer, IL-17F homodimer, and IL-17AF heterodimer. In some such embodiments, an anti-IL-17 antibody that is capable of binding IL-17A homodimer, IL-17F homodimer, and IL-17AF heterodimer can also be referred to as an IL-17A and F antibody or IL-17A and IL-17F cross-reactive antibody or IL-17A/F cross-reactive antibody. In certain such embodiments, the IL-17A and F cross-reactive antibody binds to identical or similar epitopes on IL-17A, IL-17F and/or IL-17AF heterodimer. In certain embodiments, the IL-17A and F cross-reactive antibody binds to identical or similar epitopes on IL-17A, IL-17F and/or IL-17AF heterodimer with sufficient affinity. In certain advantageous embodiments, the IL-17A and F cross-reactive antibody or the bispecific anti-IL-13/IL-17 antibody binds to IL-17A, IL-17F and IL-17AF with high affinity. The structures of IL-17A and IL-17F have been reported. See Hymowitz et al., 2001, Embo J, 20(19):5332-41, Ely et al., 2009, Nature Immunology 10(12):1245-1252, and Liu et al., 2013, Nature Communications DOI: 10.1038/ncomms2880. Similar or identical epitopes comprising amino acid resides present in the surface area of IL-17A and IL-17F can be deduced from the structures.

The terms “anti-IL-13 antibody” and “an antibody that binds to IL-13” refer to an antibody that is capable of binding IL-13 with sufficient affinity such that the antibody is useful as a diagnostic and/or therapeutic agent in targeting IL-13. In some embodiments, the extent of binding of an anti-IL-13 antibody to an unrelated, non-IL-13 protein is less than about 10% of the binding of the antibody to IL-13 as measured, e.g., by a radioimmunoassay (RIA). In certain embodiments, an antibody that binds to IL-13 has a dissociation constant (Kd) of ≦1 μM, ≦100 nM, ≦10 nM, ≦1 nM, ≦0.1 nM, ≦0.01 nM, or ≦0.001 nM (e.g. 10-8 M or less, e.g. from 10-8 M to 10-13 M, e.g., from 10-9 M to 10-13 M). In certain embodiments, an anti-IL-13 antibody binds to an epitope of IL-13 that is conserved among IL-13 from different species. In some embodiments, an anti-IL-13 antibody is a multispecific antibody, such as a bispecific antibody.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

Competition assays may be used to identify an antibody that competes with a reference antibody for binding to a target antigen. In certain embodiments, such a competing antibody binds to the same epitope (e.g., a linear or a conformational epitope) that is bound by the reference antibody. Detailed exemplary methods for mapping an epitope to which an antibody binds are provided in, e.g., Morris (1996) “Epitope Mapping Protocols,” in Methods in Molecular Biology vol. 66 (Humana Press, Totowa, N.J.).

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more (sometimes referred to as cross-blocking). An exemplary competition assay is provided herein. Suitable assays for competition analysis and epitope mapping include without limitation cross-blocking assays, competition ELISA or Biacore, NMR, and X-ray crystography.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamycin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In some embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In some embodiments, for the VL, the subgroup is subgroup kappa I as in Kabat et al., supra. In some embodiments, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-IL-17 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

“Isolated nucleic acid encoding an anti-IL-13 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some embodiments, a monoclonal antibody is a multispecific (such as bispecific) antibody.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products. The term “package insert” is also used to refer to instructions customarily included in commercial packages of diagnostic products that contain information about the intended use, test principle, preparation and handling of reagents, specimen collection and preparation, calibration of the assay and the assay procedure, performance and precision data such as sensitivity and specificity of the assay.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “IL-17” as used herein refers to IL-17A homodimer, IL-17F homodimer, and/or IL-17AF heterodimer, unless indicated otherwise. The term IL-17A, IL-17AA, IL-17AA homodimer, and IL-17A homodimer are used interchangeably, unless indicated otherwise. The terms IL-17F, IL-17FF, IL-17FF homodimer, and IL-17F homodimer are used interchangeably, unless indicated otherwise. The terms IL-17AF, IL-17AF heterodimer, and IL-17A/F heterodimer are used interchangeably, unless indicated otherwise. The term IL-17 refers to any native IL-17A, IL-17F and/or IL-17AF heterodimer from any vertebrate source, including mammals such as primates (e.g. humans) and rodents (e.g., mice and rats), unless otherwise indicated. The term encompasses “full-length,” unprocessed IL-17 as well as any form of IL-17 that results from processing in the cell. The term also encompasses naturally occurring variants of IL-17, e.g., splice variants or allelic variants. The amino acid sequences of exemplary human IL-17A are shown in SEQ ID NOs: 7 and 8. The amino acid sequences of exemplary human IL-17F are shown in SEQ ID NOs: 9 and 10. In certain embodiments, the IL-17 sequences comprises an exogenous, i.e., non-native signal peptide. In certain embodiments, the IL-17 proteins are mature proteins without a signal peptide.

The term “IL-13,” as used herein, refers to any native IL-13 from any vertebrate source, including mammals such as primates (e.g. humans) and rodents (e.g., mice and rats), unless otherwise indicated. The term encompasses “full-length,” unprocessed IL-13 as well as any form of IL-13 that results from processing in the cell. The term also encompasses naturally occurring variants of IL-13, e.g., splice variants or allelic variants. The amino acid sequences of exemplary human IL-13 are shown in SEQ ID NOs: 1 and 2, and in Swiss-Prot Accession No. P35225.2. The amino acid sequence of an exemplary cynomolgus monkey IL-13 is shown in SEQ ID NO: 4. In certain embodiments, the IL-13 sequences comprises an exogenous, i.e., non-native signal peptide. In certain embodiments, the IL-13 proteins are mature proteins without a signal peptide.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, reduction of symptoms, diminishment of any direct or indirect pathological consequences of the disease, preventing metastasis, decreasing the rate of disease progression, amelioration or palliation of the disease state, and remission or improved prognosis. In some embodiments, antibodies are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### Compositions and Methods

In certain embodiments, bispecific antibodies that bind to IL-17 and IL-13 are provided. The antibodies are useful, e.g., for the diagnosis or treatment of eosinophilic disorders, including respiratory disorders (such as asthma and IPF), neutrophilic disorders, IL-17 mediated disorders, and IL-13 mediated disorders. See e.g., US 2012/0141492, U.S. Pat. No. 8,715,669 or WO2009/136,286 (each incorporated herein by reference in its entirety).

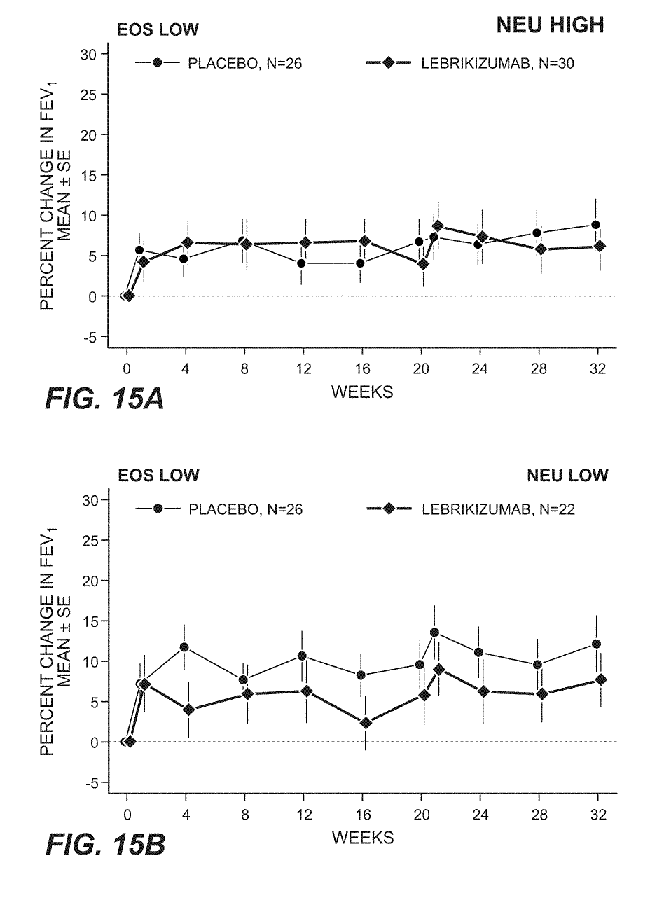

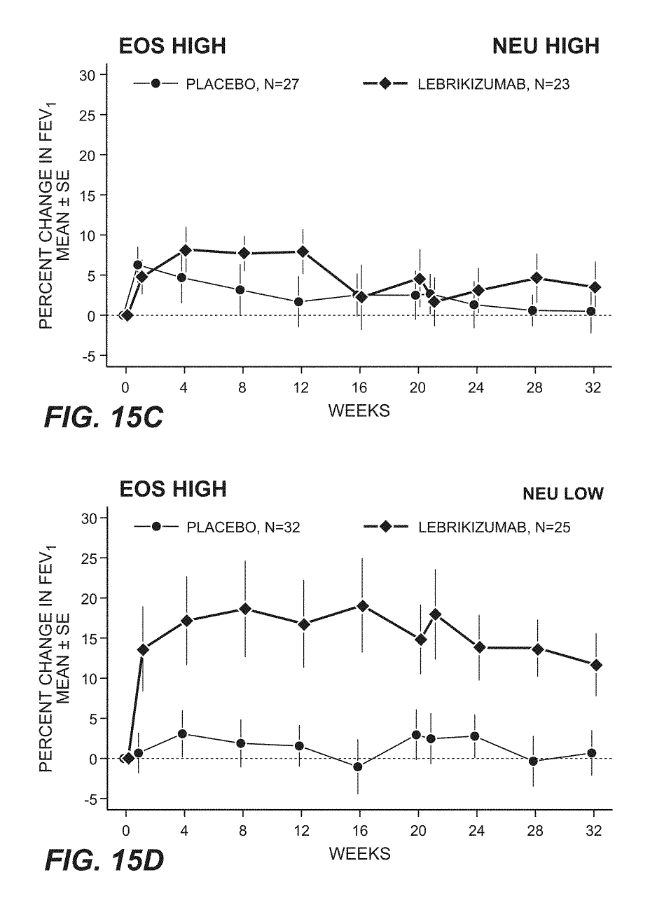

As described herein, among eosinophil-high asthma patient population, subgroups of neutrophil-high and neutrophil-low can be identified. An anti-IL13 antagonist, lebrikizumab, exhibited high treatment efficacy, as measured by ΔFEV1%, within the eosinophil-high and neutrophil-low subgroup, while lebrikizumab is less efficacious within the eosinophil-high and neutrophil-high subgroup. See FIGS. 15C and D. Thus, instead of being a district, separate group, neutrophil-high asthma may coexist with eosinophilic asthma, which further supports the advantages of the anti-IL-13/IL-17 bispecific antibody described herein and use thereof for treating moderate to severe asthma. Accordingly, in certain embodiments, the invention provides methods of treating asthma, especially moderate to severe asthma in an individual in need thereof comprising administering to the individual the multispecific antibody described herein, wherein the multispecific antibody shows improved efficacy in the individual than lebrikizumab. In some embodiments, the asthma is eosinophilic asthma, Th2-high asthma, Th2-driven asthma or IL-13-high asthma. In some embodiments, the individual has elevated blood eosinophil count and/or elevated blood neutrophil count as compared to a control or reference level. In certain embodiments, the individual has blood eosinophil count of at least 150 or at least 300 eosinophils/ul of blood. In certain embodiments, the individual has blood neutrophil count that is at least the medium blood neutrophil count of a patient population. In certain embodiments, the individual has at least 3800 neutrophils/ul of blood.

### Exemplary Anti-IL-17 Antibodies

In some embodiments, isolated antibodies that bind IL-17 are provided. In some embodiments, isolated IL-17 antibody or cross-reactive anti-IL-17A and F antibodies are provided, wherein the antibodies bind IL-17A homodimer, IL-17F homodimer, and optionally IL-17AF heterodimer. In some embodiments, isolated IL-17 antibody or cross-reactive anti-IL-17A and F antibodies bind to IL-17A homodimer, IL-17F homodimer, and IL-17AF heterodimer.

### Antibodies 15E6 and 15E6FK

In some embodiments, an anti-IL-17 antibody comprises at least one, two, three, four, five, or six HVRs selected from (a) HVR-H1 comprising the amino acid sequence of SEQ ID NO: 40; (b) HVR-H2 comprising an amino acid sequence selected from SEQ ID NOs: 41, 43, 80, 81, and 114; (c) HVR-H3 comprising the amino acid sequence of SEQ ID NO: 42 or SEQ ID NO: 44; (d) HVR-L1 comprising the amino acid sequence of SEQ ID NO: 45; (e) HVR-L2 comprising the amino acid sequence of SEQ ID NO: 46; and (f) HVR-L3 comprising the amino acid sequence of SEQ ID NO: 47.

In some embodiments, an antibody is provided that comprises at least one, at least two, or all three VH HVR sequences selected from (a) HVR-H1 comprising the amino acid sequence of SEQ ID NO: 40; (b) HVR-H2 comprising an amino acid sequence selected from SEQ ID NOs: 41, 43, 80, 81, and 114; and (c) HVR-H3 comprising the amino acid sequence of SEQ ID NO: 42 or SEQ ID NO: 44. In some embodiments, the antibody comprises HVR-H3 comprising the amino acid sequence of SEQ ID NO: 42 or SEQ ID NO: 44. In some embodiments, the antibody comprises HVR-H3 comprising the amino acid sequence of SEQ ID NO: 42 or SEQ ID NO: 44 and HVR-L3 comprising the amino acid sequence of SEQ ID NO: 47. In some embodiments, the antibody comprises HVR-H3 comprising the amino acid sequence of SEQ ID NO: 42 or SEQ ID NO: 44, HVR-L3 comprising the amino acid sequence of SEQ ID NO: 47, and HVR-H2 comprising an amino acid sequence selected from SEQ ID NOs: 41, 43, 80, 81, and 114. In some embodiments, the antibody comprises (a) HVR-H1 comprising the amino acid sequence of SEQ ID NO: 40; (b) HVR-H2 comprising an amino acid sequence selected from SEQ ID NOs: 41, 43, 80, 81, and 114; and (c) HVR-H3 comprising the amino acid sequence of SEQ ID NO: 42 or SEQ ID NO: 44.

In some embodiments, the antibody comprises (a) HVR-H1 comprising the amino acid sequence of SEQ ID NO: 40; (b) HVR-H2 comprising the amino acid sequence of SEQ ID NO: 41; and (c) HVR-H3 comprising the amino acid sequence of SEQ ID NO: 42. In some embodiments, the antibody comprises (a) HVR-H1 comprising the amino acid sequence of SEQ ID NO: 40; (b) HVR-H2 comprising the amino acid sequence of SEQ ID NO: 43; and (c) HVR-H3 comprising the amino acid sequence of SEQ ID NO: 44. In some embodiments, the antibody comprises (a) HVR-H1 comprising the amino acid sequence of SEQ ID NO: 40; (b) HVR-H2 comprising the amino acid sequence of SEQ ID NO: 80; and (c) HVR-H3 comprising the amino acid sequence of SEQ ID NO: 44. In some embodiments, the antibody comprises (a) HVR-H1 comprising the amino acid sequence of SEQ ID NO: 40; (b) HVR-H2 comprising the amino acid sequence of SEQ ID NO: 81; and (c) HVR-H3 comprising the amino acid sequence of SEQ ID NO: 44. In some embodiments, the antibody comprises (a) HVR-H1 comprising the amino acid sequence of SEQ ID NO: 40; (b) HVR-H2 comprising the amino acid sequence of SEQ ID NO: 114; and (c) HVR-H3 comprising the amino acid sequence of SEQ ID NO: 44.

In some embodiments, an antibody is provided that comprises at least one, at least two, or all three VL HVR sequences selected from (a) HVR-L1 comprising the amino acid sequence of SEQ ID NO: 45; (b) HVR-L2 comprising the amino acid sequence of SEQ ID NO: 46; and (c) HVR-L3 comprising the amino acid sequence of SEQ ID NO: 47. In some embodiments, the antibody comprises (a) HVR-L1 comprising the amino acid sequence of SEQ ID NO: 45; (b) HVR-L2 comprising the amino acid sequence of SEQ ID NO: 46; and (c) HVR-L3 comprising the amino acid sequence of SEQ ID NO: 47.

In some embodiments, an antibody comprises (a) a VH domain comprising at least one, at least two, or all three VH HVR sequences selected from (i) HVR-H1 comprising the amino acid sequence of SEQ ID NO: 40, (ii) HVR-H2 comprising an amino acid sequence selected from SEQ ID NOs: 41, 43, 80, and 81, and (iii) HVR-H3 comprising an amino acid sequence selected from SEQ ID NO: 42 or SEQ ID NO: 44; and (b) a VL domain comprising at least one, at least two, or all three VL HVR sequences selected from (i) HVR-L1 comprising the amino acid sequence of SEQ ID NO: 45, (ii) HVR-L2 comprising the amino acid sequence of SEQ ID NO: 46, and (c) HVR-L3 comprising the amino acid sequence of SEQ ID NO: 47.

In some embodiments, an antibody is provided that comprises (a) HVR-H1 comprising the amino acid sequence of SEQ ID NO: 40; (b) HVR-H2 comprising an amino acid sequence selected from SEQ ID NOs: 41, 43, 80, 81, and 114; (c) HVR-H3 comprising the amino acid sequence of SEQ ID NO: 42 or SEQ ID NO: 44; (d) HVR-L1 comprising the amino acid sequence of SEQ ID NO: 45; (e) HVR-L2 comprising the amino acid sequence of SEQ ID NO: 46; and (f) HVR-L3 comprising an amino acid sequence selected from SEQ ID NO: 47.

In some embodiments, an antibody is provided that comprises (a) HVR-H1 comprising the amino acid sequence of SEQ ID NO: 40; (b) HVR-H2 comprising the amino acid sequence of SEQ ID NO: 43; (c) HVR-H3 comprising the amino acid sequence of SEQ ID NO: 44; (d) HVR-L1 comprising the amino acid sequence of SEQ ID NO: 45; (e) HVR-L2 comprising the amino acid sequence of SEQ ID NO: 46; and (f) HVR-L3 comprising an amino acid sequence selected from SEQ ID NO: 47.

In some embodiments, an antibody is provided that comprises (a) HVR-H1 comprising the amino acid sequence of SEQ ID NO: 40; (b) HVR-H2 comprising the amino acid sequence of SEQ ID NO: 80; (c) HVR-H3 comprising the amino acid sequence of SEQ ID NO: 44; (d) HVR-L1 comprising the amino acid sequence of SEQ ID NO: 45; (e) HVR-L2 comprising the amino acid sequence of SEQ ID NO: 46; and (f) HVR-L3 comprising an amino acid sequence selected from SEQ ID NO: 47. In some embodiments, an antibody is provided that comprises (a) HVR-H1 comprising the amino acid sequence of SEQ ID NO: 40; (b) HVR-H2 comprising the amino acid sequence of SEQ ID NO: 81; (c) HVR-H3 comprising the amino acid sequence of SEQ ID NO: 44; (d) HVR-L1 comprising the amino acid sequence of SEQ ID NO: 45; (e) HVR-L2 comprising the amino acid sequence of SEQ ID NO: 46; and (f) HVR-L3 comprising an amino acid sequence selected from SEQ ID NO: 47. In some embodiments, an antibody is provided that comprises (a) HVR-H1 comprising the amino acid sequence of SEQ ID NO: 40; (b) HVR-H2 comprising the amino acid sequence of SEQ ID NO: 114; (c) HVR-H3 comprising the amino acid sequence of SEQ ID NO: 44; (d) HVR-L1 comprising the amino acid sequence of SEQ ID NO: 45; (e) HVR-L2 comprising the amino acid sequence of SEQ ID NO: 46; and (f) HVR-L3 comprising an amino acid sequence selected from SEQ ID NO: 47.

In any embodiments described herein comprising SEQ ID NO: 80, X may be any amino acid except N. In any embodiments described herein comprising SEQ ID NO: 80, X may be selected from G, A, Q, H, D, K, S, and R. In any embodiments described herein comprising SEQ ID NO: 80, X may be selected from G, A, Q, D, and S. In any embodiments described herein comprising SEQ ID NO: 80, X may be selected from D, S and Q. In any embodiments described herein comprising SEQ ID NO: 81, X may be any amino acid except S or T. In any embodiments described herein comprising SEQ ID NO: 81, X may be selected from A, G, P, N and V. In any embodiments described herein comprising SEQ ID NO: 81, X may be selected from A, G and N. In any embodiments described herein comprising SEQ ID NO: 114, X may be selected from D, S, or Q. In some embodiments, the NWS motif of SEQ ID NO: 43 is changed to NPS.

In some embodiments, an anti-IL-17 antibody comprises a heavy chain variable domain (VH) sequence having at least 90%, 91%, 92%, 93%, 94%, 95%, 96%, 97%, 98%, 99%, or 100% sequence identity to an amino acid sequence selected from SEQ ID NOs: 37, 39, 82, 83, and 115. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-17 antibody comprising that sequence retains the ability to bind to IL-17. In certain embodiments, a total of 1 to 10 amino acids have been substituted, inserted and/or deleted in SEQ ID NO: 37. In certain embodiments, a total of 1 to 10 amino acids have been substituted, inserted and/or deleted in SEQ ID NO: 39. In certain embodiments, a total of 1 to 10 amino acids have been substituted, inserted and/or deleted in SEQ ID NO: 82. In certain embodiments, a total of 1 to 10 amino acids have been substituted, inserted and/or deleted in SEQ ID NO: 83. In certain embodiments, a total of 1 to 10 amino acids have been substituted, inserted and/or deleted in SEQ ID NO: 115. In certain embodiments, substitutions, insertions, or deletions occur in regions outside the HVRs (i.e., in the FRs). Optionally, the anti-IL-17 antibody comprises the VH sequence in SEQ ID NO: 37, including post-translational modifications of that sequence. Optionally, the anti-IL-17 antibody comprises the VH sequence in SEQ ID NO: 39, including post-translational modifications of that sequence. Optionally, the anti-IL-17 antibody comprises the VH sequence in SEQ ID NO: 82, including post-translational modifications of that sequence. Optionally, the anti-IL-17 antibody comprises the VH sequence in SEQ ID NO: 83, including post-translational modifications of that sequence. Optionally, the anti-IL-17 antibody comprises the VH sequence in SEQ ID NO: 115, including post-translational modifications of that sequence. In a particular embodiment, the heavy chain variable domain comprises one, two or three HVRs selected from: (a) HVR-H1 comprising the amino acid sequence of SEQ ID NO: 40, (b) HVR-H2 comprising an amino acid sequence selected from SEQ ID NOs: 41, 43, 80, 81, and 114, and (c) HVR-H3 comprising the amino acid sequence of SEQ ID NO: 42 or SEQ ID NO: 44. In certain embodiments, the VH sequence comprises a HVR sequence having at least 60%, 64%, 65%, 68%, 70%, 75%, 80%, 82%, 85%, 88%, 90%, 92%, 93%, 94%, 100% sequence identity to an amino acid sequence selected from SEQ ID NOs:40, 41, 42, 43, 44, 80, 81, and 114, wherein the antibody retains the ability to bind IL-17. In certain embodiments, the VH sequence comprises a HVR-H2 sequence having at least 94% sequence identity to SEQ ID NO:43. In certain embodiments, the VH sequence comprises a HVR-H2 having an amino acid sequence of SEQ ID NO:43 that is not glycosylated. In certain embodiments, the VH sequence comprises a HVR-H2 having an amino acid sequence of SEQ ID NO:80, 81, or 114.

In some embodiments, an anti-IL-17 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 38. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-17 antibody comprising that sequence retains the ability to bind to IL-17. In certain embodiments, a total of 1 to 10 amino acids have been substituted, inserted and/or deleted in SEQ ID NO: 38. In certain embodiments, the substitutions, insertions, or deletions occur in regions outside the HVRs (i.e., in the FRs). Optionally, the anti-IL-17 antibody comprises the VL sequence in SEQ ID NO: 38, including post-translational modifications of that sequence. In a particular embodiment, the VL comprises one, two or three HVRs selected from (a) HVR-L1 comprising the amino acid sequence of SEQ ID NO: 45; (b) HVR-L2 comprising the amino acid sequence of SEQ ID NO: 46; and (c) HVR-L3 comprising the amino acid sequence of SEQ ID NO: 47. In certain embodiments, the VL sequence comprises a HVR sequence having at least 60%, 64%, 65%, 68%, 70%, 75%, 80%, 82%, 85%, 88%, 90%, 92%, 93%, 94%, 100% sequence identity to an amino acid sequence selected from SEQ ID NOs:45, 46, and 47, wherein the antibody retains the ability to bind IL-17.

In some embodiments, an anti-IL-17 antibody is provided, wherein the antibody comprises a VH as in any of the embodiments provided above, and a VL as in any of the embodiments provided above. In some embodiments, the antibody comprises the VH and VL sequences in SEQ ID NO: 37 and SEQ ID NO: 38, respectively, including post-translational modifications of those sequences. In some embodiments, the antibody comprises the VH and VL sequences in SEQ ID NO: 39 and SEQ ID NO: 38, respectively, including post-translational modifications of those sequences. In some embodiments, the antibody comprises the VH and VL sequences in SEQ ID NO: 82 and SEQ ID NO: 38, respectively, including post-translational modifications of those sequences. In some embodiments, the antibody comprises the VH and VL sequences in SEQ ID NO: 83 and SEQ ID NO: 38, respectively, including post-translational modifications of those sequences. In some embodiments, the antibody comprises the VH and VL sequences in SEQ ID NO: 115 and SEQ ID NO: 38, respectively, including post-translational modifications of those sequences.

In any embodiments described herein comprising SEQ ID NO: 82, X may be any amino acid except N. In any embodiments described herein comprising SEQ ID NO: 82, X may be selected from G, A, Q, H, D, K, and R. In any embodiments described herein comprising SEQ ID NO: 82, X may be selected from G, A, and Q. In any embodiments described herein comprising SEQ ID NO: 83, X may be any amino acid except S or T. In any embodiments described herein comprising SEQ ID NO: 83, X may be A, G, P or V. In any embodiments described herein comprising SEQ ID NO: 115, X may be D, S, or Q.

In some embodiments, an antibody is provided that competes for binding to IL-17 with an anti-IL-17 antibody comprising a VH sequence of SEQ ID NO: 39 and a VL sequence of SEQ ID NO: 38. In some embodiments, an antibody is provided that binds to the same epitope as an anti-IL-17 antibody provided herein. For example, in certain embodiments, an antibody is provided that binds to the same epitope as an anti-IL-17 antibody comprising a VH sequence of SEQ ID NO: 39 and a VL sequence of SEQ ID NO: 38.

In some embodiments, an anti-IL-17 antibody according to any of the above embodiments is a monoclonal antibody, including a chimeric, humanized or human antibody. In some embodiments, an anti-IL-17 antibody is an antibody fragment, e.g., a Fv, Fab, Fab′, scFv, diabody, or F(ab′)₂ fragment. In some embodiments, the antibody is a full length antibody, e.g., an intact IgG1 or IgG4 antibody or other antibody class or isotype as defined herein.

In some embodiments, an anti-IL-17 antibody according to any of the above embodiments may incorporate any of the features, singly or in combination, as described in Sections 1-7 below.

### Antibodies 30D12 and 30D12BF

In some embodiments, an anti-IL-17 antibody comprises at least one, two, three, four, five, or six HVRs selected from (a) HVR-H1 comprising the amino acid sequence of SEQ ID NO: 51; (b) HVR-H2 comprising the amino acid sequence of SEQ ID NO: 52 or SEQ ID NO: 53; (c) HVR-H3 comprising the amino acid sequence of SEQ ID NO: 54; (d) HVR-L1 comprising the amino acid sequence of SEQ ID NO: 55; (e) HVR-L2 comprising the amino acid sequence of SEQ ID NO: 56; and (f) HVR-L3 comprising the amino acid sequence of SEQ ID NO: 57.

In some embodiments, an antibody is provided that comprises at least one, at least two, or all three VH HVR sequences selected from (a) HVR-H1 comprising the amino acid sequence of SEQ ID NO: 51; (b) HVR-H2 comprising the amino acid sequence of SEQ ID NO: 52 or SEQ ID NO: 53; and (c) HVR-H3 comprising the amino acid sequence of SEQ ID NO: 54. In some embodiments, the antibody comprises HVR-H3 comprising the amino acid sequence of SEQ ID NO: 54. In some embodiments, the antibody comprises HVR-H3 comprising the amino acid sequence of SEQ ID NO: 54 and HVR-L3 comprising the amino acid sequence of SEQ ID NO: 57. In some embodiments, the antibody comprises HVR-H3 comprising the amino acid sequence of SEQ ID NO: 54, HVR-L3 comprising the amino acid sequence of SEQ ID NO: 57, and HVR-H2 comprising the amino acid sequence of SEQ ID NO: 52 or SEQ ID NO: 53. In some embodiments, the antibody comprises (a) HVR-H1 comprising the amino acid sequence of SEQ ID NO: 51; (b) HVR-H2 comprising the amino acid sequence of SEQ ID NO: 52 or SEQ ID NO: 53; and (c) HVR-H3 comprising the amino acid sequence of SEQ ID NO: 54. In some embodiments, the antibody comprises (a) HVR-H1 comprising the amino acid sequence of SEQ ID NO: 51; (b) HVR-H2 comprising the amino acid sequence of SEQ ID NO: 52; and (c) HVR-H3 comprising the amino acid sequence of SEQ ID NO: 54. In some embodiments, the antibody comprises (a) HVR-H1 comprising the amino acid sequence of SEQ ID NO: 51; (b) HVR-H2 comprising the amino acid sequence of SEQ ID NO: 53; and (c) HVR-H3 comprising the amino acid sequence of SEQ ID NO: 54.

In some embodiments, an antibody is provided that comprises at least one, at least two, or all three VL HVR sequences selected from (a) HVR-L1 comprising the amino acid sequence of SEQ ID NO: 55; (b) HVR-L2 comprising the amino acid sequence of SEQ ID NO: 56; and (c) HVR-L3 comprising the amino acid sequence of SEQ ID NO: 57. In some embodiments, the antibody comprises (a) HVR-L1 comprising the amino acid sequence of SEQ ID NO: 55; (b) HVR-L2 comprising the amino acid sequence of SEQ ID NO: 56; and (c) HVR-L3 comprising the amino acid sequence of SEQ ID NO: 57.

In some embodiments, an antibody comprises (a) a VH domain comprising at least one, at least two, or all three VH HVR sequences selected from (i) HVR-H1 comprising the amino acid sequence of SEQ ID NO: 51, (ii) HVR-H2 comprising the amino acid sequence of SEQ ID NO: 52 or SEQ ID NO: 53, and (iii) HVR-H3 comprising an amino acid sequence selected from SEQ ID NO: 54; and (b) a VL domain comprising at least one, at least two, or all three VL HVR sequences selected from (i) HVR-L1 comprising the amino acid sequence of SEQ ID NO: 55, (ii) HVR-L2 comprising the amino acid sequence of SEQ ID NO: 56, and (c) HVR-L3 comprising the amino acid sequence of SEQ ID NO: 57.

In some embodiments, an antibody is provided that comprises (a) HVR-H1 comprising the amino acid sequence of SEQ ID NO: 51; (b) HVR-H2 comprising the amino acid sequence of SEQ ID NO: 52 or SEQ ID NO: 53; (c) HVR-H3 comprising the amino acid sequence of SEQ ID NO: 54; (d) HVR-L1 comprising the amino acid sequence of SEQ ID NO: 55; (e) HVR-L2 comprising the amino acid sequence of SEQ ID NO: 56; and (f) HVR-L3 comprising an amino acid sequence selected from SEQ ID NO: 57. In some embodiments, an antibody is provided that comprises (a) HVR-H1 comprising the amino acid sequence of SEQ ID NO: 51; (b) HVR-H2 comprising the amino acid sequence of SEQ ID NO: 53; (c) HVR-H3 comprising the amino acid sequence of SEQ ID NO: 54; (d) HVR-L1 comprising the amino acid sequence of SEQ ID NO: 55; (e) HVR-L2 comprising the amino acid sequence of SEQ ID NO: 56; and (f) HVR-L3 comprising an amino acid sequence selected from SEQ ID NO: 57.

In some embodiments, an anti-IL-17 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 48 or SEQ ID NO: 50. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-17 antibody comprising that sequence retains the ability to bind to IL-17. In certain embodiments, a total of 1 to 10 amino acids have been substituted, inserted and/or deleted in SEQ ID NO: 48. In certain embodiments, a total of 1 to 10 amino acids have been substituted, inserted and/or deleted in SEQ ID NO: 50. In certain embodiments, substitutions, insertions, or deletions occur in regions outside the HVRs (i.e., in the FRs). Optionally, the anti-IL-17 antibody comprises the VH sequence in SEQ ID NO: 48, including post-translational modifications of that sequence. Optionally, the anti-IL-17 antibody comprises the VH sequence in SEQ ID NO: 50, including post-translational modifications of that sequence. In a particular embodiment, the VH comprises one, two or three HVRs selected from: (a) HVR-H1 comprising the amino acid sequence of SEQ ID NO: 51, (b) HVR-H2 comprising the amino acid sequence of SEQ ID NO: 52 or SEQ ID NO: 53, and (c) HVR-H3 comprising the amino acid sequence of SEQ ID NO: 54. In certain embodiments, the VH sequence comprises a HVR sequence having at least 60%, 64%, 65%, 68%, 70%, 75%, 80%, 82%, 85%, 88%, 90%, 92%, 93%, 94%, 100% sequence identity to an amino acid sequence selected from SEQ ID NOs:51, 52, 53, and 54, wherein the antibody retains the ability to bind IL-17.

In some embodiments, an anti-IL-17 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 49. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-17 antibody comprising that sequence retains the ability to bind to IL-17. In certain embodiments, a total of 1 to 10 amino acids have been substituted, inserted and/or deleted in SEQ ID NO: 49. In certain embodiments, the substitutions, insertions, or deletions occur in regions outside the HVRs (i.e., in the FRs). Optionally, the anti-IL-17 antibody comprises the VL sequence in SEQ ID NO: 49, including post-translational modifications of that sequence. In a particular embodiment, the VL comprises one, two or three HVRs selected from (a) HVR-L1 comprising the amino acid sequence of SEQ ID NO: 55; (b) HVR-L2 comprising the amino acid sequence of SEQ ID NO: 56; and (c) HVR-L3 comprising the amino acid sequence of SEQ ID NO: 57. In certain embodiments, the VL sequence comprises a HVR sequence having at least 60%, 64%, 65%, 68%, 70%, 75%, 80%, 82%, 85%, 88%, 90%, 92%, 93%, 94%, 100% sequence identity to an amino acid sequence selected from SEQ ID NOs:55, 56, and 57, wherein the antibody retains the ability to bind IL-17.

In some embodiments, an anti-IL-17 antibody is provided, wherein the antibody comprises a VH as in any of the embodiments provided above, and a VL as in any of the embodiments provided above. In some embodiments, the antibody comprises the VH and VL sequences in SEQ ID NO: 48 and SEQ ID NO: 49, respectively, including post-translational modifications of those sequences. In some embodiments, the antibody comprises the VH and VL sequences in SEQ ID NO: 50 and SEQ ID NO: 49, respectively, including post-translational modifications of those sequences.

In some embodiments, an antibody is provided that competes for binding to IL-17 with an anti-IL-17 antibody comprising a VH sequence of SEQ ID NO: 50 and a VL sequence of SEQ ID NO: 49. In some embodiments, an antibody is provided that binds to the same epitope as an anti-IL-17 antibody provided herein. For example, in certain embodiments, an antibody is provided that binds to the same epitope as an anti-IL-17 antibody comprising a VH sequence of SEQ ID NO: 50 and a VL sequence of SEQ ID NO: 49.

In some embodiments, an anti-IL-17 antibody according to any of the above embodiments is a monoclonal antibody, including a chimeric, humanized or human antibody. In some embodiments, an anti-IL-17 antibody is an antibody fragment, e.g., a Fv, Fab, Fab′, scFv, diabody, or F(ab′)₂ fragment. In some embodiments, the antibody is a full length antibody, e.g., an intact IgG1 or IgG4 antibody or other antibody class or isotype as defined herein.

In some embodiments, an anti-IL-17 antibody according to any of the above embodiments may incorporate any of the features, singly or in combination, as described in Sections 1-7 below.

### Antibodies 39F12 and 39F12A

In some embodiments, an anti-IL-17 antibody comprises at least one, two, three, four, five, or six HVRs selected from (a) HVR-H1 comprising the amino acid sequence of SEQ ID NO: 61; (b) HVR-H2 comprising the amino acid sequence of SEQ ID NO: 62; (c) HVR-H3 comprising the amino acid sequence of SEQ ID NO: 63; (d) HVR-L1 comprising the amino acid sequence of SEQ ID NO: 64; (e) HVR-L2 comprising the amino acid sequence of SEQ ID NO: 65; and (f) HVR-L3 comprising the amino acid sequence of SEQ ID NO: 66.

In some embodiments, an antibody is provided that comprises at least one, at least two, or all three VH HVR sequences selected from (a) HVR-H1 comprising the amino acid sequence of SEQ ID NO: 61; (b) HVR-H2 comprising the amino acid sequence of SEQ ID NO: 62; and (c) HVR-H3 comprising the amino acid sequence of SEQ ID NO: 63. In some embodiments, the antibody comprises HVR-H3 comprising the amino acid sequence of SEQ ID NO: 63. In some embodiments, the antibody comprises HVR-H3 comprising the amino acid sequence of SEQ ID NO: 63 and HVR-L3 comprising the amino acid sequence of SEQ ID NO: 66. In some embodiments, the antibody comprises HVR-H3 comprising the amino acid sequence of SEQ ID NO: 63, HVR-L3 comprising the amino acid sequence of SEQ ID NO: 66, and HVR-H2 comprising the amino acid sequence of SEQ ID NO: 62. In some embodiments, the antibody comprises (a) HVR-H1 comprising the amino acid sequence of SEQ ID NO: 61; (b) HVR-H2 comprising the amino acid sequence of SEQ ID NO: 62; and (c) HVR-H3 comprising the amino acid sequence of SEQ ID NO: 63.

In some embodiments, an antibody is provided that comprises at least one, at least two, or all three VL HVR sequences selected from (a) HVR-L1 comprising the amino acid sequence of SEQ ID NO: 64; (b) HVR-L2 comprising the amino acid sequence of SEQ ID NO: 65; and (c) HVR-L3 comprising the amino acid sequence of SEQ ID NO: 66. In some embodiments, the antibody comprises (a) HVR-L1 comprising the amino acid sequence of SEQ ID NO: 64; (b) HVR-L2 comprising the amino acid sequence of SEQ ID NO: 65; and (c) HVR-L3 comprising the amino acid sequence of SEQ ID NO: 66.

In some embodiments, an antibody comprises (a) a VH domain comprising at least one, at least two, or all three VH HVR sequences selected from (i) HVR-H1 comprising the amino acid sequence of SEQ ID NO: 61, (ii) HVR-H2 comprising the amino acid sequence of SEQ ID NO: 62, and (iii) HVR-H3 comprising an amino acid sequence selected from SEQ ID NO: 63; and (b) a VL domain comprising at least one, at least two, or all three VL HVR sequences selected from (i) HVR-L1 comprising the amino acid sequence of SEQ ID NO: 64, (ii) HVR-L2 comprising the amino acid sequence of SEQ ID NO: 65, and (c) HVR-L3 comprising the amino acid sequence of SEQ ID NO: 66.

In some embodiments, an antibody is provided that comprises (a) HVR-H1 comprising the amino acid sequence of SEQ ID NO: 61; (b) HVR-H2 comprising the amino acid sequence of SEQ ID NO: 62; (c) HVR-H3 comprising the amino acid sequence of SEQ ID NO: 63; (d) HVR-L1 comprising the amino acid sequence of SEQ ID NO: 64; (e) HVR-L2 comprising the amino acid sequence of SEQ ID NO: 65; and (f) HVR-L3 comprising an amino acid sequence selected from SEQ ID NO: 66.

In some embodiments, an anti-IL-17 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 58. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-17 antibody comprising that sequence retains the ability to bind to IL-17. In certain embodiments, a total of 1 to 10 amino acids have been substituted, inserted and/or deleted in SEQ ID NO: 58. In certain embodiments, substitutions, insertions, or deletions occur in regions outside the HVRs (i.e., in the FRs). Optionally, the anti-IL-17 antibody comprises the VH sequence in SEQ ID NO: 58, including post-translational modifications of that sequence. In a particular embodiment, the VH comprises one, two or three HVRs selected from: (a) HVR-H1 comprising the amino acid sequence of SEQ ID NO: 61, (b) HVR-H2 comprising the amino acid sequence of SEQ ID NO: 62, and (c) HVR-H3 comprising the amino acid sequence of SEQ ID NO: 63. In certain embodiments, the VH sequence comprises a HVR sequence having at least 60%, 64%, 65%, 68%, 70%, 75%, 80%, 82%, 85%, 88%, 90%, 92%, 93%, 94%, 100% sequence identity to an amino acid sequence selected from SEQ ID NOs:61, 62, and 63, wherein the antibody retains the ability to bind IL-17.

In some embodiments, an anti-IL-17 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 59 or SEQ ID NO: 60. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-17 antibody comprising that sequence retains the ability to bind to IL-17. In certain embodiments, a total of 1 to 10 amino acids have been substituted, inserted and/or deleted in SEQ ID NO: 59. In certain embodiments, a total of 1 to 10 amino acids have been substituted, inserted and/or deleted in SEQ ID NO: 60. In certain embodiments, the substitutions, insertions, or deletions occur in regions outside the HVRs (i.e., in the FRs). Optionally, the anti-IL-17 antibody comprises the VL sequence in SEQ ID NO: 59, including post-translational modifications of that sequence. Optionally, the anti-IL-17 antibody comprises the VL sequence in SEQ ID NO: 60, including post-translational modifications of that sequence. In a particular embodiment, the VL comprises one, two or three HVRs selected from (a) HVR-L1 comprising the amino acid sequence of SEQ ID NO: 64; (b) HVR-L2 comprising the amino acid sequence of SEQ ID NO: 65; and (c) HVR-L3 comprising the amino acid sequence of SEQ ID NO: 66. In certain embodiments, the VL sequence comprises a HVR sequence having at least 60%, 64%, 65%, 68%, 70%, 75%, 80%, 82%, 85%, 88%, 90%, 92%, 93%, 94%, 100% sequence identity to an amino acid sequence selected from SEQ ID NOs:64, 65 and 66, wherein the antibody retains the ability to bind IL-17.

In some embodiments, an anti-IL-17 antibody is provided, wherein the antibody comprises a VH as in any of the embodiments provided above, and a VL as in any of the embodiments provided above. In some embodiments, the antibody comprises the VH and VL sequences in SEQ ID NO: 58 and SEQ ID NO: 59, respectively, including post-translational modifications of those sequences. In some embodiments, the antibody comprises the VH and VL sequences in SEQ ID NO: 58 and SEQ ID NO: 60, respectively, including post-translational modifications of those sequences.

In some embodiments, an antibody is provided that competes for binding to IL-17 with an anti-IL-17 antibody comprising a VH sequence of SEQ ID NO: 58 and a VL sequence of SEQ ID NO: 60. In some embodiments, an antibody is provided that binds to the same epitope as an anti-IL-17 antibody provided herein. For example, in certain embodiments, an antibody is provided that binds to the same epitope as an anti-IL-17 antibody comprising a VH sequence of SEQ ID NO: 58 and a VL sequence of SEQ ID NO: 60.

In some embodiments, an anti-IL-17 antibody according to any of the above embodiments is a monoclonal antibody, including a chimeric, humanized or human antibody. In some embodiments, an anti-IL-17 antibody is an antibody fragment, e.g., a Fv, Fab, Fab′, scFv, diabody, or F(ab′)₂ fragment. In some embodiments, the antibody is a full length antibody, e.g., an intact IgG1 or IgG4 antibody or other antibody class or isotype as defined herein.

In some embodiments, an anti-IL-17 antibody according to any of the above embodiments may incorporate any of the features, singly or in combination, as described in Sections 1-7 below.

### Exemplary Anti-IL-13 Antibodies

In some embodiments, isolated antibodies that bind IL-13 are provided. In some embodiments, an anti-IL-13 antibody comprises at least one, two, three, four, five, or six HVRs selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17; (d) HVR-L1 comprising the amino acid sequence of SEQ ID NO: 18; (e) HVR-L2 comprising the amino acid sequence of SEQ ID NO: 19; and (f) HVR-L3 comprising the amino acid sequence of SEQ ID NO: 20. See e.g., U.S. Pat. No. 8,088,618 and WO2005/062967 (each incorporated herein by reference in its entirety).

In some embodiments, an antibody is provided that comprises at least one, at least two, or all three VH HVR sequences selected from (a) HVR-H1 comprising the amino acid sequence of SEQ ID NO: 15; (b) HVR-H2 comprising the amino acid sequence of SEQ ID NO: 16; and (c) HVR-H3 comprising the amino acid sequence of SEQ ID NO: 17. In some embodiments, the antibody comprises HVR-H3 comprising the amino acid sequence of SEQ ID NO: 17. In some embodiments, the antibody comprises HVR-H3 comprising the amino acid sequence of SEQ ID NO: 17 and HVR-L3 comprising the amino acid sequence of SEQ ID NO: 20. In some embodiments, the antibody comprises HVR-H3 comprising the amino acid sequence of SEQ ID NO: 17, HVR-L3 comprising the amino acid sequence of SEQ ID NO: 20, and HVR-H2 comprising the amino acid sequence of SEQ ID NO: 16. In some embodiments, the antibody comprises (a) HVR-H1 comprising the amino acid sequence of SEQ ID NO: 15; (b) HVR-H2 comprising the amino acid sequence of SEQ ID NO: 16; and (c) HVR-H3 comprising the amino acid sequence of SEQ ID NO: 17.

In some embodiments, an antibody is provided that comprises at least one, at least two, or all three VL HVR sequences selected from (a) HVR-L1 comprising the amino acid sequence of SEQ ID NO: 18; (b) HVR-L2 comprising the amino acid sequence of SEQ ID NO: 19; and (c) HVR-L3 comprising the amino acid sequence of SEQ ID NO: 20. In some embodiments, the antibody comprises (a) HVR-L1 comprising the amino acid sequence of SEQ ID NO: 18; (b) HVR-L2 comprising the amino acid sequence of SEQ ID NO: 19; and (c) HVR-L3 comprising the amino acid sequence of SEQ ID NO: 20.

In some embodiments, an antibody comprises (a) a VH domain comprising at least one, at least two, or all three VH HVR sequences selected from (i) HVR-H1 comprising the amino acid sequence of SEQ ID NO: 15, (ii) HVR-H2 comprising the amino acid sequence of SEQ ID NO: 16, and (iii) HVR-H3 comprising an amino acid sequence selected from SEQ ID NO: 17; and (b) a VL domain comprising at least one, at least two, or all three VL HVR sequences selected from (i) HVR-L1 comprising the amino acid sequence of SEQ ID NO: 18, (ii) HVR-L2 comprising the amino acid sequence of SEQ ID NO: 19, and (c) HVR-L3 comprising the amino acid sequence of SEQ ID NO: 20.

In some embodiments, an antibody is provided that comprises (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17; (d) HVR-L1 comprising the amino acid sequence of SEQ ID NO: 18; (e) HVR-L2 comprising the amino acid sequence of SEQ ID NO: 19; and (f) HVR-L3 comprising an amino acid sequence selected from SEQ ID NO: 20.

In any of the above embodiments, an anti-IL-13 antibody is humanized. In some embodiments, an anti-IL-13 antibody comprises HVRs as in any of the above embodiments, and further comprises an acceptor human framework, e.g. a human immunoglobulin framework or a human consensus framework. In some embodiments, an anti-IL-13 antibody comprises HVRs as in any of the above embodiments, and further comprises a VH comprising FR1, FR2, FR3, and/or FR4 sequences of SEQ ID NO: 13. In some embodiments, an anti-IL-13 antibody comprises HVRs as in any of the above embodiments, and further comprises a VL comprising FR1, FR2, FR3, and/or FR4 sequences of SEQ ID NO: 14. In some embodiments, an anti-IL-13 antibody comprises HVRs as in any of the above embodiments, and further comprises a VH comprising FR1, FR2, FR3, and/or FR4 sequences of SEQ ID NO: 11. In some embodiments, an anti-IL-13 antibody comprises HVRs as in any of the above embodiments, and further comprises a VL comprising FR1, FR2, FR3, and/or FR4 sequences of SEQ ID NO: 12.

In some embodiments, the binding of anti-IL13 antibody to IL-13 inhibits the intracellular signaling of IL-13 mediated by IL-13Rα1/IL-4Rα. In some such embodiments, the anti-IL13 antibody does not inhibit binding of IL-13 to IL-13Rα1. In some such embodiments, the anti-IL-13 antibody inhibits binding of IL-13 to IL-4Rα. In some embodiments, the anti-IL-13 antibody is lebrikizumab. See Ultsch et al., 2013, J. Mol. Biol. 425:1330-1339. In some embodiments, the anti-IL-13 antibody contains the M4L substitution in the light chain (SEQ ID NO:14) and the Q1E subsitutin in the heavy chain (SEQ ID NO:13).

In some embodiments, an anti-IL-13 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 13. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-13 antibody comprising that sequence retains the ability to bind to IL-13. In certain embodiments, a total of 1 to 10 amino acids have been substituted, inserted and/or deleted in SEQ ID NO: 13. In certain embodiments, substitutions, insertions, or deletions occur in regions outside the HVRs (i.e., in the FRs). In some embodiments, the anti-IL-13 antibody comprises the VH sequence in SEQ ID NO: 13, including post-translational modifications of that sequence. In some embodiments, the anti-IL-13 antibody comprises the VH sequence in SEQ ID NO: 11, including post-translational modifications of that sequence. In some embodiments, the VH comprises one, two or three HVRs selected from: (a) HVR-H1 comprising the amino acid sequence of SEQ ID NO: 15, (b) HVR-H2 comprising the amino acid sequence of SEQ ID NO: 16, and (c) HVR-H3 comprising the amino acid sequence of SEQ ID NO: 17.

In some embodiments, an anti-IL-13 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 14. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-13 antibody comprising that sequence retains the ability to bind to IL-13. In certain embodiments, a total of 1 to 10 amino acids have been substituted, inserted and/or deleted in SEQ ID NO: 14. In certain embodiments, the substitutions, insertions, or deletions occur in regions outside the HVRs (i.e., in the FRs). In some embodiments, the anti-IL-13 antibody comprises the VL sequence in SEQ ID NO: 14, including post-translational modifications of that sequence. In some embodiments, the anti-IL-13 antibody comprises the VL sequence in SEQ ID NO: 12, including post-translational modifications of that sequence. In some embodiments, the VL comprises one, two or three HVRs selected from (a) HVR-L1 comprising the amino acid sequence of SEQ ID NO: 18; (b) HVR-L2 comprising the amino acid sequence of SEQ ID NO: 19; and (c) HVR-L3 comprising the amino acid sequence of SEQ ID NO: 20.

In certain embodiments, the VH sequence comprises a HVR sequence having at least 60%, 64%, 65%, 68%, 70%, 75%, 80%, 82%, 85%, 88%, 90%, 92%, 93%, 94%, 100% sequence identity to an amino acid sequence selected from SEQ ID NOs:15, 16 and 17, wherein the antibody retains the ability to bind IL-13. In certain embodiments, the VL sequence comprises a HVR sequence having at least 60%, 64%, 65%, 68%, 70%, 75%, 80%, 82%, 85%, 88%, 90%, 92%, 93%, 94%, 100% sequence identity to an amino acid sequence selected from SEQ ID NOs:18, 19 and 20, wherein the antibody retains the ability to bind IL-13.

In some embodiments, an anti-IL-13 antibody is provided, wherein the antibody comprises a VH as in any of the embodiments provided above, and a VL as in any of the embodiments provided above. In some embodiments, the antibody comprises the VH sequence in SEQ ID NO: 13 or SEQ ID NO: 11 and the VL sequence in SEQ ID NO: 14 or SEQ ID NO: 12, including post-translational modifications of those sequences.

In some embodiments, an antibody is provided that competes for binding to IL-13 with an anti-IL-13 antibody comprising a VH sequence of SEQ ID NO: 13 and a VL sequence of SEQ ID NO: 14. In some embodiments, an antibody is provided that binds to the same epitope as an anti-IL-13 antibody provided herein. See, e.g., Ultsch, M. et al., Structural Basis of Signaling Blockade by Anti-IL-13 Antibody Lebrikizumab, J. Mol. Biol. (2013), dx.doi.org/10.1016/j.jmb.2013.01.024. In some embodiments, an antibody is provided that binds to the same epitope as an anti-IL-13 antibody provided herein. For example, in certain embodiments, an antibody is provided that binds to the same epitope as an anti-IL-13 antibody comprising a VH sequence of SEQ ID NO: 13 and a VL sequence of SEQ ID NO: 14. In certain embodiments, an antibody is provided that binds to an epitope within amino acids 77 to 89 of IL-13 (SEQ ID NO: 1), which are YCAALESLINVSG (SEQ ID NO: 6). In certain embodiments, an antibody is provided that binds to an epitope within amino acids 82 to 89 of IL-13 (SEQ ID NO: 1), which are ESLINVSG (SEQ ID NO: 5).

Another exemplary anti-IL-13 antibody is 11H4 and humanized versions thereof, including hu11H4v6. Mu11H4 comprises heavy chain and light chain variable regions comprising the amino acid sequences of SEQ ID NOs: 26 and 25, respectively. Humanized hu11H4v6 comprises a heavy chain variable region and a light chain variable region comprising the amino acid sequence of SEQ ID NOs: 30 and 29, respectively. Humanized hu11H4v6 comprises a heavy chain and a light chain comprising the amino acid sequence of SEQ ID NOs: 28 and 27, respectively.

In some embodiments, an anti-IL-13 antibody comprises at least one, two, three, four, five, or six HVRs selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33; (d) HVR-L1 comprising the amino acid sequence of SEQ ID NO: 34; (e) HVR-L2 comprising the amino acid sequence of SEQ ID NO: 35; and (f) HVR-L3 comprising the amino acid sequence of SEQ ID NO: 36.

In some embodiments, an antibody is provided that comprises at least one, at least two, or all three VH HVR sequences selected from (a) HVR-H1 comprising the amino acid sequence of SEQ ID NO: 31; (b) HVR-H2 comprising the amino acid sequence of SEQ ID NO: 32; and (c) HVR-H3 comprising the amino acid sequence of SEQ ID NO: 33. In some embodiments, the antibody comprises HVR-H3 comprising the amino acid sequence of SEQ ID NO: 33. In some embodiments, the antibody comprises HVR-H3 comprising the amino acid sequence of SEQ ID NO: 33 and HVR-L3 comprising the amino acid sequence of SEQ ID NO: 36. In some embodiments, the antibody comprises HVR-H3 comprising the amino acid sequence of SEQ ID NO: 33, HVR-L3 comprising the amino acid sequence of SEQ ID NO: 36, and HVR-H2 comprising the amino acid sequence of SEQ ID NO: 32. In some embodiments, the antibody comprises (a) HVR-H1 comprising the amino acid sequence of SEQ ID NO: 31; (b) HVR-H2 comprising the amino acid sequence of SEQ ID NO: 32; and (c) HVR-H3 comprising the amino acid sequence of SEQ ID NO: 33.

In some embodiments, an antibody is provided that comprises at least one, at least two, or all three VL HVR sequences selected from (a) HVR-L1 comprising the amino acid sequence of SEQ ID NO: 34; (b) HVR-L2 comprising the amino acid sequence of SEQ ID NO: 35; and (c) HVR-L3 comprising the amino acid sequence of SEQ ID NO: 36. In some embodiments, the antibody comprises (a) HVR-L1 comprising the amino acid sequence of SEQ ID NO: 34; (b) HVR-L2 comprising the amino acid sequence of SEQ ID NO: 35; and (c) HVR-L3 comprising the amino acid sequence of SEQ ID NO: 36.

In some embodiments, an antibody comprises (a) a VH domain comprising at least one, at least two, or all three VH HVR sequences selected from (i) HVR-H1 comprising the amino acid sequence of SEQ ID NO: 31, (ii) HVR-H2 comprising the amino acid sequence of SEQ ID NO: 32, and (iii) HVR-H3 comprising an amino acid sequence selected from SEQ ID NO: 33; and (b) a VL domain comprising at least one, at least two, or all three VL HVR sequences selected from (i) HVR-L1 comprising the amino acid sequence of SEQ ID NO: 34, (ii) HVR-L2 comprising the amino acid sequence of SEQ ID NO: 35, and (c) HVR-L3 comprising the amino acid sequence of SEQ ID NO: 36.

In some embodiments, an antibody is provided that comprises (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33; (d) HVR-L1 comprising the amino acid sequence of SEQ ID NO: 34; (e) HVR-L2 comprising the amino acid sequence of SEQ ID NO: 35; and (f) HVR-L3 comprising an amino acid sequence selected from SEQ ID NO: 36.

In any of the above embodiments, an anti-IL-13 antibody is humanized. In some embodiments, an anti-IL-13 antibody comprises HVRs as in any of the above embodiments, and further comprises an acceptor human framework, e.g. a human immunoglobulin framework or a human consensus framework. In some embodiments, an anti-IL-13 antibody comprises HVRs as in any of the above embodiments, and further comprises a VH comprising FR1, FR2, FR3, and/or FR4 sequences of SEQ ID NO: 30. In some embodiments, an anti-IL-13 antibody comprises HVRs as in any of the above embodiments, and further comprises a VL comprising FR1, FR2, FR3, and/or FR4 sequences of SEQ ID NO: 29.

In some embodiments, an anti-IL-13 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 30. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-13 antibody comprising that sequence retains the ability to bind to IL-13. In certain embodiments, a total of 1 to 10 amino acids have been substituted, inserted and/or deleted in SEQ ID NO: 30. In certain embodiments, substitutions, insertions, or deletions occur in regions outside the HVRs (i.e., in the FRs). Optionally, the anti-IL-13 antibody comprises the VH sequence in SEQ ID NO: 30, including post-translational modifications of that sequence. In a particular embodiment, the VH comprises one, two or three HVRs selected from: (a) HVR-H1 comprising the amino acid sequence of SEQ ID NO: 31, (b) HVR-H2 comprising the amino acid sequence of SEQ ID NO: 32, and (c) HVR-H3 comprising the amino acid sequence of SEQ ID NO: 33.

In some embodiments, an anti-IL-13 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 29. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-13 antibody comprising that sequence retains the ability to bind to IL-13. In certain embodiments, a total of 1 to 10 amino acids have been substituted, inserted and/or deleted in SEQ ID NO: 29. In certain embodiments, the substitutions, insertions, or deletions occur in regions outside the HVRs (i.e., in the FRs). Optionally, the anti-IL-13 antibody comprises the VL sequence in SEQ ID NO: 29, including post-translational modifications of that sequence. In a particular embodiment, the VL comprises one, two or three HVRs selected from (a) HVR-L1 comprising the amino acid sequence of SEQ ID NO: 34; (b) HVR-L2 comprising the amino acid sequence of SEQ ID NO: 35; and (c) HVR-L3 comprising the amino acid sequence of SEQ ID NO: 36.

In certain embodiments, the VH sequence comprises a HVR sequence having at least 60%, 64%, 65%, 68%, 70%, 75%, 80%, 82%, 85%, 88%, 90%, 92%, 93%, 94%, 100% sequence identity to an amino acid sequence selected from SEQ ID NOs: 31, 32, and 33, wherein the antibody retains the ability to bind IL-13. In certain embodiments, the VL sequence comprises a HVR sequence having at least 60%, 64%, 65%, 68%, 70%, 75%, 80%, 82%, 85%, 88%, 90%, 92%, 93%, 94%, 100% sequence identity to an amino acid sequence selected from SEQ ID NOs: 34, 35 and 36, wherein the antibody retains the ability to bind IL-13.

In some embodiments, an anti-IL-13 antibody is provided, wherein the antibody comprises a VH as in any of the embodiments provided above, and a VL as in any of the embodiments provided above. In some embodiments, the antibody comprises the VH and VL sequences in SEQ ID NO: 30 and SEQ ID NO: 29, respectively, including post-translational modifications of those sequences.

In some embodiments, an antibody is provided that competes for binding to IL-13 with an anti-IL-13 antibody comprising a VH sequence of SEQ ID NO: 30 and a VL sequence of SEQ ID NO: 29. In some embodiments, an antibody is provided that binds to the same epitope as an anti-IL-13 antibody provided herein. See, e.g., Ultsch, M. et al., Structural Basis of Signaling Blockade by Anti-IL-13 Antibody Lebrikizumab, J. Mol. Biol. (2013), dx.doi.org/10.1016/j.jmb.2013.01.053. In some embodiments, an antibody is provided that binds to the same epitope as an anti-IL-13 antibody provided herein. For example, in certain embodiments, an antibody is provided that binds to the same epitope as an anti-IL-13 antibody comprising a VH sequence of SEQ ID NO: 30 and a VL sequence of SEQ ID NO: 29.

In some embodiments, an anti-IL-13 antibody according to any of the above embodiments is a monoclonal antibody, including a chimeric, humanized or human antibody. In some embodiments, an anti-IL-13 antibody is an antibody fragment, e.g., a Fv, Fab, Fab′, scFv, diabody, or F(ab′)₂ fragment. In some embodiments, the antibody is a full length antibody, e.g., an intact IgG1 or IgG4 antibody or other antibody class or isotype as defined herein.

In some embodiments, an anti-IL-13 antibody according to any of the above embodiments may incorporate any of the features, singly or in combination, as described in Sections 1-7 below.

### Exemplary Anti-IL-13/IL-17 Bispecific Antibodies

In some embodiments, a multispecific antibody (such as a bispecific antibody) comprising an antigen-binding domain that specifically binds to IL-17 and IL-13 is provided. In some embodiments, the antigen-binding domain does not specifically bind to other targets. The multispecific antibody that binds IL-17 and IL-13 may comprise a first set of variable regions (VH and VL; also referred to as a VH/VL unit) according to any of the embodiments described herein for anti-IL-17 antibodies, and a second set of variable regions (VH and VL; also referred to as a VH/VL unit) according to any of the embodiments described herein for anti-IL-13 antibodies. In some embodiments, the anti-IL-13/IL-17 bispecific antibody comprises (i) a first half antibody comprising the first VH/VL unit and at least a portion of a heavy chain constant region and/or at least a portion of a light chain constant region, and (ii) a second half antibody comprising the second VH/VL unit and at least a portion of a heavy chain constant region and/or at least a portion of a light chain constant region. In some embodiments, the first half antibody binds IL-17 but does not bind to IL-13, and the second half antibody binds IL-13 but does not bind IL-17. In some embodiments, the multispecific antibody maintains the natural antibody format and is not a dual variable domain (DVD) antibody. See, e.g., PCT Publication No. 2013/102042. WO2013/102042 describes a dual specific antigen binding protein to IL-13 and IL-17A that is a bivalent binder to IL-13 and a bivalent binder to IL-17A, which may contribute to binding avidity to each target. In some embodiments of the invention, the anti-IL-13/IL-17 bispecific antibody is a monovalent binder to IL-13 and a monovalent binder to IL-17AA, AF and FF. As discussed herein, it was further discovered that the bispecific antibody in which each half antibody comprises a monovalent binder to IL-13 and IL-17 (IL-17AA, AF, FF), respectively, maintains comparable binding activities and potencies as compared to each of the parent bivalent monospecific antibodies. As further described below, in some embodiments, the anti-IL-13/IL-17 bispecific antibody comprises a first VH/VL unit and a second VH/VL unit, wherein the first VH/VL unit binds to IL-17 and comprises HVR-H1 comprising the amino acid sequence of SEQ ID NO: 40, HVR-H2 comprising an amino acid sequence selected from SEQ ID NOs: 43, HVR-H3 comprising the amino acid sequence of SEQ ID NO: 44, HVR-L1 comprising the amino acid sequence of SEQ ID NO: 45, HVR-L2 comprising the amino acid sequence of SEQ ID NO: 46, and HVR-L3 comprising the amino acid sequence of SEQ ID NO: 47, and wherein the second VH/VL unit binds IL-13 and comprises HVR-H1 comprising the amino acid sequence of SEQ ID NO: 15, HVR-H2 comprising the amino acid sequence of SEQ ID NO: 16, HVR-H3 comprising the amino acid sequence of SEQ ID NO: 17, HVR-L1 comprising the amino acid sequence of SEQ ID NO: 18, HVR-L2 comprising the amino acid sequence of SEQ ID NO: 19, and HVR-L3 comprising the amino acid sequence of SEQ ID NO: 20, and wherein the IL-13/IL-17 bispecific antibody binds and inhibits IL-13 and IL-17AA, AF and FF.

### Multispecific Antibodies Comprising 15E6 or 15E6FK

In some embodiments, the multispecific antibody comprises an antigen-binding domain that specifically binds to IL-17 and IL-13 where the antibody comprises a first VH/VL unit comprising a VH (heavy chain variable domain) comprising an amino acid sequence selected from SEQ ID NOs: 37, 39, 82, 83, and 115. In some embodiments, the multispecific antibody comprises an antigen-binding domain that specifically binds to IL-17 and IL-13 where the antibody comprises a first VH/VL unit comprising a VL (light chain variable domain) comprising the amino acid sequence of SEQ ID NO: 38.

In some embodiments, the multispecific antibody comprises an antigen-binding domain that specifically binds to IL-17 and IL-13 where the antibody comprises a first VH/VL unit comprising a VH comprising the amino acid sequence of SEQ ID NO: 37 and a VL comprising the amino acid sequence of SEQ ID NO: 38. In some embodiments, the multispecific antibody comprises an antigen-binding domain that specifically binds to IL-17 and IL-13 where the antibody comprises a first VH/VL unit comprising a VH comprising the amino acid sequence of SEQ ID NO: 39 and a VL comprising the amino acid sequence of SEQ ID NO: 38. In some embodiments, the multispecific antibody comprises an antigen-binding domain that specifically binds to IL-17 and IL-13 where the antibody comprises a first VH/VL unit comprising a VH comprising the amino acid sequence of SEQ ID NO: 82 and a VL comprising the amino acid sequence of SEQ ID NO: 38. In some embodiments, the multispecific antibody comprises an antigen-binding domain that specifically binds to IL-17 and IL-13 where the antibody comprises a first VH/VL unit comprising a VH comprising the amino acid sequence of SEQ ID NO: 83 and a VL comprising the amino acid sequence of SEQ ID NO: 38. In some embodiments, the multispecific antibody comprises an antigen-binding domain that specifically binds to IL-17 and IL-13 where the antibody comprises a first VH/VL unit comprising a VH comprising the amino acid sequence of SEQ ID NO: 115 and a VL comprising the amino acid sequence of SEQ ID NO: 38.

In some embodiments, the multispecific antibody comprises an antigen-binding domain that specifically binds to IL-17 and IL-13 where the antibody comprises a first VH/VL unit that competes for binding to IL-17 with an antibody comprising a VH comprising the amino acid sequence of SEQ ID NO: 37 and a VL comprising the amino acid sequence of SEQ ID NO: 38.

In some embodiments, the multispecific antibody comprises an antigen-binding domain that specifically binds to IL-17 and IL-13 where the antibody comprises a second VH/VL unit comprising a VH (heavy chain variable domain) comprising the amino acid sequence of SEQ ID NO: 13 or SEQ ID NO: 11. In some embodiments, the multispecific antibody comprises an antigen-binding domain that specifically binds to IL-17 and IL-13 where the antibody comprises a second VH/VL unit comprising a VL (light chain variable domain) comprising the amino acid sequence of SEQ ID NO: 14 or SEQ ID NO: 12. In some embodiments, the multispecific antibody comprises an antigen-binding domain that specifically binds to IL-17 and IL-13 where the antibody comprises a second VH/VL unit comprising a VH comprising the amino acid sequence of SEQ ID NO: 13 or SEQ ID NO: 11 and a VL comprising the amino acid sequence of SEQ ID NO: 14 or SEQ ID NO: 12. In some embodiments, the multispecific antibody comprises an antigen-binding domain that specifically binds to IL-17 and IL-13 where the antibody comprises a second VH/VL unit that competes for binding to IL-13 with an antibody comprising a VH comprising the amino acid sequence of SEQ ID NO: 13 and a VL comprising the amino acid sequence of SEQ ID NO: 14. In some embodiments, the multispecific antibody comprises an antigen-binding domain that specifically binds to IL-17 and IL-13 where the antibody comprises a second VH/VL unit that binds an epitope of IL-13 consisting of amino acids 82 to 89 of SEQ ID NO: 1. In some embodiments, the multispecific antibody comprises an antigen-binding domain that specifically binds to IL-17 and IL-13 where the antibody comprises a second VH/VL unit that binds an epitope of IL-13 consisting of amino acids 77 to 89 of SEQ ID NO: 1.

In some embodiments, the multispecific antibody comprises an antigen-binding domain that specifically binds to IL-17 and IL-13 where the antibody comprises a second VH/VL unit comprising a VH (heavy chain variable domain) comprising the amino acid sequence of SEQ ID NO: 30. In some embodiments, the multispecific antibody comprises an antigen-binding domain that specifically binds to IL-17 and IL-13 where the antibody comprises a second VH/VL unit comprising a VL (light chain variable domain) comprising the amino acid sequence of SEQ ID NO: 29. In some embodiments, the multispecific antibody comprises an antigen-binding domain that specifically binds to IL-17 and IL-13 where the antibody comprises a second VH/VL unit comprising a VH comprising the amino acid sequence of SEQ ID NO: 30 and a VL comprising the amino acid sequence of SEQ ID NO: 29. In some embodiments, the multispecific antibody comprises an antigen-binding domain that specifically binds to IL-17 and IL-13 where the antibody comprises a second VH/VL unit that competes for binding to IL-13 with an antibody comprising a VH comprising the amino acid sequence of SEQ ID NO: 30 and a VL comprising the amino acid sequence of SEQ ID NO: 29.

In some embodiments, the multispecific antibody comprises an antigen-binding domain that specifically binds to IL-17 and IL-13 where the antibody comprises a first VH/VL unit comprising a first VH comprising an amino acid sequence selected from SEQ ID NOs: 37, 39, 82, 83, and 115 and a first VL comprising the amino acid sequence of SEQ ID NO: 38; and comprises a second VH/VL unit comprising a second VH comprising the amino acid sequence of SEQ ID NO: 13 or SEQ ID NO: 11 and a second VL comprising the amino acid sequence of SEQ ID NO: 14 or SEQ ID NO: 12.

In some embodiments, the multispecific antibody comprises an antigen-binding domain that specifically binds to IL-17 and IL-13 where the antibody comprises a first VH/VL unit comprising a first VH comprising an amino acid sequence selected from SEQ ID NOs: 37, 39, 82, 83, and 115 and a first VL comprising the amino acid sequence of SEQ ID NO: 38; and comprises a second VH/VL unit comprising a second VH comprising the amino acid sequence of SEQ ID NO: 30 and a second VL comprising the amino acid sequence of SEQ ID NO: 29.

In some embodiments, the multispecific antibody comprises an antigen-binding domain that specifically binds to IL-17 and IL-13 wherein the antibody comprises a first VH/VL unit comprising a VH having at least 90%, 91%, 92%, 93%, 94%, 95%, 96%, 97%, 98%, 99%, or 100% sequence identity to an amino acid sequence selected from SEQ ID NOs: 37, 39, 82, 83, and 115 and a VL having at least 90%, 91%, 92%, 93%, 94%, 95%, 96%, 97%, 98%, 99%, or 100% sequence identity to the amino acid sequence of SEQ ID NO: 38. In certain embodiments, a total of 1 to 10 amino acids have been substituted, inserted and/or deleted in the sequences above. In certain embodiments, substitutions, insertions, or deletions occur in regions outside the HVRs (i.e., in the FRs).

In some embodiments, the multispecific antibody comprises an antigen-binding domain that specifically binds to IL-17 and IL-13 where the antibody comprises a second VH/VL unit comprising a VH having at least 90%, 91%, 92%, 93%, 94%, 95%, 96%, 97%, 98%, 99%, or 100% sequence identity to the amino acid sequence of SEQ ID NO: 13 and a VL having at least 90%, 91%, 92%, 93%, 94%, 95%, 96%, 97%, 98%, 99%, or 100% sequence identity to the amino acid sequence of SEQ ID NO: 14. In some embodiments, the multispecific antibody comprises an antigen-binding domain that specifically binds to IL-17 and IL-13 where the antibody comprises a second VH/VL unit comprising a VH having at least 90%, 91%, 92%, 93%, 94%, 95%, 96%, 97%, 98%, 99%, or 100% sequence identity to the amino acid sequence of SEQ ID NO: 30 and a VL having at least 90%, 91%, 92%, 93%, 94%, 95%, 96%, 97%, 98%, 99%, or 100% sequence identity to the amino acid sequence of SEQ ID NO: 29. In certain embodiments, a total of 1 to 10 amino acids have been substituted, inserted and/or deleted in the sequences above. In certain embodiments, substitutions, insertions, or deletions occur in regions outside the HVRs (i.e., in the FRs).

In some embodiments, the multispecific antibody comprises an antigen-binding domain that specifically binds to IL-17 and IL-13 wherein the antibody comprises a first VH/VL unit comprising a first VH having at least 90%, 91%, 92%, 93%, 94%, 95%, 96%, 97%, 98%, 99%, or 100% sequence identity to an amino acid sequence selected from SEQ ID NOs: 37, 39, 82, 83, and 115 and a VL having at least 90%, 91%, 92%, 93%, 94%, 95%, 96%, 97%, 98%, 99%, or 100% sequence identity to the amino acid sequence of SEQ ID NO: 38; and a second VH/VL unit comprising a second VH having at least 90%, 91%, 92%, 93%, 94%, 95%, 96%, 97%, 98%, 99%, or 100% sequence identity to the amino acid sequence of SEQ ID NO: 13 and a second VL having at least 90%, 91%, 92%, 93%, 94%, 95%, 96%, 97%, 98%, 99%, or 100% sequence identity to the amino acid sequence of SEQ ID NO: 14. In certain embodiments, a total of 1 to 10 amino acids have been substituted, inserted and/or deleted in the sequences above. In certain embodiments, substitutions, insertions, or deletions occur in regions outside the HVRs (i.e., in the FRs).

In some embodiments, the multispecific antibody comprises an antigen-binding domain that specifically binds to IL-17 and IL-13 wherein the antibody comprises a first VH/VL unit comprising a first VH having at least 90%, 91%, 92%, 93%, 94%, 95%, 96%, 97%, 98%, 99%, or 100% sequence identity to an amino acid sequence selected from SEQ ID NOs: 37, 39, 82, 83, and 115 and a VL having at least 90%, 91%, 92%, 93%, 94%, 95%, 96%, 97%, 98%, 99%, or 100% sequence identity to the amino acid sequence of SEQ ID NO: 38; and a second VH/VL unit comprising a second VH having at least 90%, 91%, 92%, 93%, 94%, 95%, 96%, 97%, 98%, 99%, or 100% sequence identity to the amino acid sequence of SEQ ID NO: 30 and a second VL having at least 90%, 91%, 92%, 93%, 94%, 95%, 96%, 97%, 98%, 99%, or 100% sequence identity to the amino acid sequence of SEQ ID NO: 29. In certain embodiments, a total of 1 to 10 amino acids have been substituted, inserted and/or deleted in the sequences above. In certain embodiments, substitutions, insertions, or deletions occur in regions outside the HVRs (i.e., in the FRs).

In some embodiments, a multispecific antibody comprises an antigen-binding domain that specifically binds to IL-17 and IL-13 where the antibody comprises a first VH/VL unit comprising at least one, two, three, four, five, or six HVRs selected from (a) HVR-H1 comprising the amino acid sequence of SEQ ID NO: 40; (b) HVR-H2 comprising an amino acid sequence selected from SEQ ID NOs: 41, 43, 80, 81, and 114; (c) HVR-H3 comprising the amino acid sequence of SEQ ID NO: 42 or SEQ ID NO: 44; (d) HVR-L1 comprising the amino acid sequence of SEQ ID NO: 45; (e) HVR-L2 comprising the amino acid sequence of SEQ ID NO: 46; and (f) HVR-L3 comprising the amino acid sequence of SEQ ID NO: 47.

In some embodiments, a multispecific antibody comprises an antigen-binding domain that specifically binds to IL-17 and IL-13 where the antibody comprises a second VH/VL unit comprising at least one, two, three, four, five, or six HVRs selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17; (d) HVR-L1 comprising the amino acid sequence of SEQ ID NO: 18; (e) HVR-L2 comprising the amino acid sequence of SEQ ID NO: 19; and (f) HVR-L3 comprising the amino acid sequence of SEQ ID NO: 20. In some embodiments, a multispecific antibody comprises an antigen-binding domain that specifically binds to IL-17 and IL-13 where the antibody comprises a second VH/VL unit comprising at least one, two, three, four, five, or six HVRs selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33; (d) HVR-L1 comprising the amino acid sequence of SEQ ID NO: 34; (e) HVR-L2 comprising the amino acid sequence of SEQ ID NO: 35; and (f) HVR-L3 comprising the amino acid sequence of SEQ ID NO: 36.

In some embodiments, a multispecific antibody comprises an antigen-binding domain that specifically binds to IL-17 and IL-13 where the antibody comprises a first VH/VL unit comprising at least one, two, three, four, five, or six HVRs selected from (a) HVR-H1 comprising the amino acid sequence of SEQ ID NO: 40; (b) HVR-H2 comprising an amino acid sequence selected from SEQ ID NOs: 41, 43, 80, 81, and 114; (c) HVR-H3 comprising the amino acid sequence of SEQ ID NO: 42 or SEQ ID NO: 44; (d) HVR-L1 comprising the amino acid sequence of SEQ ID NO: 45; (e) HVR-L2 comprising the amino acid sequence of SEQ ID NO: 46; and (f) HVR-L3 comprising the amino acid sequence of SEQ ID NO: 47; and a second VH/VL unit comprising at least one, two, three, four, five, or six HVRs selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17; (d) HVR-L1 comprising the amino acid sequence of SEQ ID NO: 18; (e) HVR-L2 comprising the amino acid sequence of SEQ ID NO: 19; and (f) HVR-L3 comprising the amino acid sequence of SEQ ID NO: 20.

In some embodiments, a multispecific antibody comprises an antigen-binding domain that specifically binds to IL-17 and IL-13 where the antibody comprises a first VH/VL unit comprising at least one, two, three, four, five, or six HVRs selected from (a) HVR-H1 comprising the amino acid sequence of SEQ ID NO: 40; (b) HVR-H2 comprising an amino acid sequence selected from SEQ ID NOs: 41, 43, 80, 81, and 114; (c) HVR-H3 comprising the amino acid sequence of SEQ ID NO: 42 or SEQ ID NO: 44; (d) HVR-L1 comprising the amino acid sequence of SEQ ID NO: 45; (e) HVR-L2 comprising the amino acid sequence of SEQ ID NO: 46; and (f) HVR-L3 comprising the amino acid sequence of SEQ ID NO: 47; and a second VH/VL unit comprising at least one, two, three, four, five, or six HVRs selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33; (d) HVR-L1 comprising the amino acid sequence of SEQ ID NO: 34; (e) HVR-L2 comprising the amino acid sequence of SEQ ID NO: 35; and (f) HVR-L3 comprising the amino acid sequence of SEQ ID NO: 36.

In some embodiments, a multispecific antibody comprises an antigen-binding domain that specifically binds to IL-17 and IL-13 where the antibody comprises a first VH/VL unit comprising at least one, at least two, or all three VH HVR sequences selected from (a) HVR-H1 comprising the amino acid sequence of SEQ ID NO: 40; (b) HVR-H2 comprising an amino acid sequence selected from SEQ ID NOs: 41, 43, 80, 81, and 114; and (c) HVR-H3 comprising the amino acid sequence of SEQ ID NO: 42 or SEQ ID NO: 44.

In some embodiments, a multispecific antibody comprises an antigen-binding domain that specifically binds to IL-17 and IL-13 where the antibody comprises a second VH/VL unit comprising at least one, at least two, or all three VH HVR sequences selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17. In some embodiments, a multispecific antibody comprises an antigen-binding domain that specifically binds to IL-17 and IL-13 where the antibody comprises a second VH/VL unit comprising at least one, at least two, or all three VH HVR sequences selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33.

In some embodiments, a multispecific antibody comprises an antigen-binding domain that specifically binds to IL-17 and IL-13 where the antibody comprises a first VH/VL unit comprising at least one, at least two, or all three VH HVR sequences selected from (a) HVR-H1 comprising the amino acid sequence of SEQ ID NO: 40; (b) HVR-H2 comprising an amino acid sequence selected from SEQ ID NOs: 41, 43, 80, 81, and 114; and (c) HVR-H3 comprising the amino acid sequence of SEQ ID NO: 42 or SEQ ID NO: 44; and a second VH/VL unit comprising at least one, at least two, or all three VH HVR sequences selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17. In some embodiments, a multispecific antibody comprises an antigen-binding domain that specifically binds to IL-17 and IL-13 where the antibody comprises a first VH/VL unit comprising at least one, at least two, or all three VH HVR sequences selected from (a) HVR-H1 comprising the amino acid sequence of SEQ ID NO: 40; (b) HVR-H2 comprising an amino acid sequence selected from SEQ ID NOs: 41, 43, 80, 81, and 114; and (c) HVR-H3 comprising the amino acid sequence of SEQ ID NO: 42 or SEQ ID NO: 44; and a second VH/VL unit comprising at least one, at least two, or all three VH HVR sequences selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33.

In some embodiments, a multispecific antibody comprises an antigen-binding domain that specifically binds to IL-17 and IL-13 where the antibody comprises a first VH/VL unit comprising at least one, at least two, or all three VL HVR sequences selected from (a) HVR-L1 comprising the amino acid sequence of SEQ ID NO: 45; (b) HVR-L2 comprising the amino acid sequence of SEQ ID NO: 46; and (c) HVR-L3 comprising the amino acid sequence of SEQ ID NO: 47.

In some embodiments, a multispecific antibody comprises an antigen-binding domain that specifically binds to IL-17 and IL-13 where the antibody comprises a second VH/VL unit comprising at least one, at least two, or all three VL HVR sequences selected from (a) HVR-L1 comprising the amino acid sequence of SEQ ID NO: 18; (b) HVR-L2 comprising the amino acid sequence of SEQ ID NO: 19; and (c) HVR-L3 comprising the amino acid sequence of SEQ ID NO: 20. In some embodiments, a multispecific antibody comprises an antigen-binding domain that specifically binds to IL-17 and IL-13 where the antibody comprises a second VH/VL unit comprising at least one, at least two, or all three VL HVR sequences selected from (a) HVR-L1 comprising the amino acid sequence of SEQ ID NO: 34; (b) HVR-L2 comprising the amino acid sequence of SEQ ID NO: 35; and (c) HVR-L3 comprising the amino acid sequence of SEQ ID NO: 36.

In some embodiments, a multispecific antibody comprises an antigen-binding domain that specifically binds to IL-17 and IL-13 where the antibody comprises a first VH/VL unit comprising at least one, at least two, or all three VL HVR sequences selected from (a) HVR-L1 comprising the amino acid sequence of SEQ ID NO: 45; (b) HVR-L2 comprising the amino acid sequence of SEQ ID NO: 46; and (c) HVR-L3 comprising the amino acid sequence of SEQ ID NO: 47; and a second VH/VL unit comprising at least one, at least two, or all three VL HVR sequences selected from (a) HVR-L1 comprising the amino acid sequence of SEQ ID NO: 18; (b) HVR-L2 comprising the amino acid sequence of SEQ ID NO: 19; and (c) HVR-L3 comprising the amino acid sequence of SEQ ID NO: 20. In some embodiments, a multispecific antibody comprises an antigen-binding domain that specifically binds to IL-17 and IL-13 where the antibody comprises a first VH/VL unit comprising at least one, at least two, or all three VL HVR sequences selected from (a) HVR-L1 comprising the amino acid sequence of SEQ ID NO: 45; (b) HVR-L2 comprising the amino acid sequence of SEQ ID NO: 46; and (c) HVR-L3 comprising the amino acid sequence of SEQ ID NO: 47; and a second VH/VL unit comprising at least one, at least two, or all three VL HVR sequences selected from (a) HVR-L1 comprising the amino acid sequence of SEQ ID NO: 34; (b) HVR-L2 comprising the amino acid sequence of SEQ ID NO: 35; and (c) HVR-L3 comprising the amino acid sequence of SEQ ID NO: 36.

In some embodiments, a multispecific antibody comprises an antigen-binding domain that specifically binds to IL-17 and IL-13 where the antibody comprises a first VH/VL unit comprising three VH HVR sequences selected from (a) HVR-H1 comprising the amino acid sequence of SEQ ID NO: 40; (b) HVR-H2 comprising an amino acid sequence selected from SEQ ID NOs: 41, 43, 80, 81, and 114; (c) HVR-H3 comprising the amino acid sequence of SEQ ID NO: 42 or SEQ ID NO: 44; and three VL HVR sequences selected from (d) HVR-L1 comprising the amino acid sequence of SEQ ID NO: 45; (e) HVR-L2 comprising the amino acid sequence of SEQ ID NO: 46; and (f) HVR-L3 comprising an amino acid sequence selected from SEQ ID NO: 47.

In some embodiments, a multispecific antibody comprises an antigen-binding domain that specifically binds to IL-17 and IL-13 where the antibody comprises a second VH/VL unit comprising three VH HVR sequences selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17; and three VL HVR sequences selected from (a) HVR-L1 comprising the amino acid sequence of SEQ ID NO: 18; (b) HVR-L2 comprising the amino acid sequence of SEQ ID NO: 19; and (c) HVR-L3 comprising the amino acid sequence of SEQ ID NO: 20.

In some embodiments, a multispecific antibody comprises an antigen-binding domain that specifically binds to IL-17 and IL-13 where the antibody comprises a second VH/VL unit comprising three VH HVR sequences selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33; and three VL HVR sequences selected from (a) HVR-L1 comprising the amino acid sequence of SEQ ID NO: 34; (b) HVR-L2 comprising the amino acid sequence of SEQ ID NO: 35; and (c) HVR-L3 comprising the amino acid sequence of SEQ ID NO: 36.

In some embodiments, a multispecific antibody comprises an antigen-binding domain that specifically binds to IL-17 and IL-13 where the antibody comprises a first VH/VL unit comprising three VH HVR sequences selected from (a) HVR-H1 comprising the amino acid sequence of SEQ ID NO: 40; (b) HVR-H2 comprising an amino acid sequence selected from SEQ ID NOs: 41, 43, 80, 81, and 114; (c) HVR-H3 comprising the amino acid sequence of SEQ ID NO: 42 or SEQ ID NO: 44; and three VL HVR sequences selected from (d) HVR-L1 comprising the amino acid sequence of SEQ ID NO: 45; (e) HVR-L2 comprising the amino acid sequence of SEQ ID NO: 46; and (f) HVR-L3 comprising an amino acid sequence selected from SEQ ID NO: 47; and a second VH/VL unit comprising three VH HVR sequences selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17; and three VL HVR sequences selected from (a) HVR-L1 comprising the amino acid sequence of SEQ ID NO: 18; (b) HVR-L2 comprising the amino acid sequence of SEQ ID NO: 19; and (c) HVR-L3 comprising the amino acid sequence of SEQ ID NO: 20.

In some embodiments, a multispecific antibody comprises an antigen-binding domain that specifically binds to IL-17 and IL-13 where the antibody comprises a first VH/VL unit comprising three VH HVR sequences selected from (a) HVR-H1 comprising the amino acid sequence of SEQ ID NO: 40; (b) HVR-H2 comprising an amino acid sequence selected from SEQ ID NOs: 41, 43, 80, 81, and 114; (c) HVR-H3 comprising the amino acid sequence of SEQ ID NO: 42 or SEQ ID NO: 44; and three VL HVR sequences selected from (d) HVR-L1 comprising the amino acid sequence of SEQ ID NO: 45; (e) HVR-L2 comprising the amino acid sequence of SEQ ID NO: 46; and (f) HVR-L3 comprising an amino acid sequence selected from SEQ ID NO: 47; and a second VH/VL unit comprising three VH HVR sequences selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33; and three VL HVR sequences selected from (a) HVR-L1 comprising the amino acid sequence of SEQ ID NO: 34; (b) HVR-L2 comprising the amino acid sequence of SEQ ID NO: 35; and (c) HVR-L3 comprising the amino acid sequence of SEQ ID NO: 36.

In various embodiments, a multispecific antibody comprises a first hemimer comprising a first VH/VL unit that binds IL-17, wherein the first hemimer comprises a knob mutation in the heavy chain constant region, and a second hemimer comprising a second VH/VL unit that binds IL-13, wherein the second hemimer comprises a hole mutation in the heavy chain constant region. In various embodiments, a multispecific antibody comprises a first hemimer comprising a first VH/VL unit that binds IL-17, wherein the first hemimer comprises a hole mutation in the heavy chain constant region, and a second hemimer comprising a second VH/VL unit that binds IL-13, wherein the second hemimer comprises a knob mutation in the heavy chain constant region. In some embodiments, a heavy chain constant region comprising a hole mutation has the sequence shown in SEQ ID NO: 68 (IgG1) or SEQ ID NO: 70 (IgG4). In some embodiments, a heavy chain constant region comprising a knob mutation has the sequence shown in SEQ ID NO: 67 (IgG1) or SEQ ID NO: 69 (IgG4). In some embodiments, a multispecific antibody comprises a first hemimer comprising a first heavy chain having an amino acid sequence selected from SEQ ID NOs: 71, 72, 84, and 85, and a first light chain having the sequence of SEQ ID NO: 73, and a second hemimer comprising a second heavy chain having the sequence of SEQ ID NO: 21 or 23 and a second light chain having the sequence of SEQ ID NO: 22 or 24. In some embodiments, a multispecific antibody comprises a first hemimer comprising a first heavy chain having an amino acid sequence selected from SEQ ID NOs: 71, 72, 84, and 85, and a first light chain having the sequence of SEQ ID NO: 73, and a second hemimer comprising a second heavy chain having the sequence of SEQ ID NO: 21 and a second light chain having the sequence of SEQ ID NO: 22.

In any embodiments described herein comprising SEQ ID NO: 80, 82, or 84, X may be any amino acid except N. In any embodiments described herein comprising SEQ ID NO: 80, 82, or 84, X may be selected from A, G, Q, H, D, K, and R. In any embodiments described herein comprising SEQ ID NO: 80, 82, or 84, X may be selected from A, G, and Q. In any embodiments described herein comprising SEQ ID NO: 81, 83, or 85, X may be any amino acid except S or T. In any embodiments described herein comprising SEQ ID NO: 81, 83 or 85, X may be A, G, P or V. In any embodiments described herein comprising SEQ ID NO: 114 or 115, X may be D, S, or Q.

In some embodiments, an anti-IL-13/IL-17 multispecific antibody according to any of the above embodiments is a monoclonal antibody, including a chimeric, humanized or human antibody. In some embodiments, an anti-IL-13/IL-17 multispecific antibody is an antibody fragment, e.g., a Fv, Fab, Fab′, scFv, diabody, or F(ab′)₂ fragment. In some embodiments, the antibody is a full length antibody, e.g., an intact IgG1 or IgG4 antibody or other antibody class or isotype as defined herein.

In some embodiments, an anti-IL-13/IL-17 multispecific antibody according to any of the above embodiments may incorporate any of the features, singly or in combination, as described in Sections 1-7 below.

### Multispecific Antibodies Comprising 30D12 or 30D12BF

In some embodiments, the multispecific antibody comprises an antigen-binding domain that specifically binds to IL-17 and IL-13 where the antibody comprises a first VH/VL unit comprising a VH (heavy chain variable domain) comprising an amino acid sequence selected from SEQ ID NOs: 48 and 50. In some embodiments, the multispecific antibody comprises an antigen-binding domain that specifically binds to IL-17 and IL-13 where the antibody comprises a first VH/VL unit comprising a VL (light chain variable domain) comprising the amino acid sequence of SEQ ID NO: 49.

In some embodiments, the multispecific antibody comprises an antigen-binding domain that specifically binds to IL-17 and IL-13 where the antibody comprises a first VH/VL unit comprising a VH comprising the amino acid sequence of SEQ ID NO: 48 and a VL comprising the amino acid sequence of SEQ ID NO: 49. In some embodiments, the multispecific antibody comprises an antigen-binding domain that specifically binds to IL-17 and IL-13 where the antibody comprises a first VH/VL unit comprising a VH comprising the amino acid sequence of SEQ ID NO: 50 and a VL comprising the amino acid sequence of SEQ ID NO: 49.

In some embodiments, the multispecific antibody comprises an antigen-binding domain that specifically binds to IL-17 and IL-13 where the antibody comprises a first VH/VL unit that competes for binding to IL-17 with an antibody comprising a VH comprising the amino acid sequence of SEQ ID NO: 50 and a VL comprising the amino acid sequence of SEQ ID NO: 49.

In some embodiments, the multispecific antibody comprises an antigen-binding domain that specifically binds to IL-17 and IL-13 where the antibody comprises a second VH/VL unit comprising a VH (heavy chain variable domain) comprising the amino acid sequence of SEQ ID NO: 13 or SEQ ID NO: 11. In some embodiments, the multispecific antibody comprises an antigen-binding domain that specifically binds to IL-17 and IL-13 where the antibody comprises a second VH/VL unit comprising a VL (light chain variable domain) comprising the amino acid sequence of SEQ ID NO: 14 or SEQ ID NO: 12. In some embodiments, the multispecific antibody comprises an antigen-binding domain that specifically binds to IL-17 and IL-13 where the antibody comprises a second VH/VL unit comprising a VH comprising the amino acid sequence of SEQ ID NO: 13 or SEQ ID NO: 11 and a VL comprising the amino acid sequence of SEQ ID NO: 14 or SEQ ID NO: 12. In some embodiments, the multispecific antibody comprises an antigen-binding domain that specifically binds to IL-17 and IL-13 where the antibody comprises a second VH/VL unit that competes for binding to IL-13 with an antibody comprising a VH comprising the amino acid sequence of SEQ ID NO: 13 and a VL comprising the amino acid sequence of SEQ ID NO: 14. In some embodiments, the multispecific antibody comprises an antigen-binding domain that specifically binds to IL-17 and IL-13 where the antibody comprises a second VH/VL unit that binds an epitope of IL-13 consisting of amino acids 82 to 89 of SEQ ID NO: 1. In some embodiments, the multispecific antibody comprises an antigen-binding domain that specifically binds to IL-17 and IL-13 where the antibody comprises a second VH/VL unit that binds an epitope of IL-13 consisting of amino acids 77 to 89 of SEQ ID NO: 1.

In some embodiments, the multispecific antibody comprises an antigen-binding domain that specifically binds to IL-17 and IL-13 where the antibody comprises a second VH/VL unit comprising a VH (heavy chain variable domain) comprising the amino acid sequence of SEQ ID NO: 30. In some embodiments, the multispecific antibody comprises an antigen-binding domain that specifically binds to IL-17 and IL-13 where the antibody comprises a second VH/VL unit comprising a VL (light chain variable domain) comprising the amino acid sequence of SEQ ID NO: 29. In some embodiments, the multispecific antibody comprises an antigen-binding domain that specifically binds to IL-17 and IL-13 where the antibody comprises a second VH/VL unit comprising a VH comprising the amino acid sequence of SEQ ID NO: 30 and a VL comprising the amino acid sequence of SEQ ID NO: 29. In some embodiments, the multispecific antibody comprises an antigen-binding domain that specifically binds to IL-17 and IL-13 where the antibody comprises a second VH/VL unit that competes for binding to IL-13 with an antibody comprising a VH comprising the amino acid sequence of SEQ ID NO: 30 and a VL comprising the amino acid sequence of SEQ ID NO: 29.

In some embodiments, the multispecific antibody comprises an antigen-binding domain that specifically binds to IL-17 and IL-13 where the antibody comprises a first VH/VL unit comprising a first VH comprising the amino acid sequence of SEQ ID NO: 48 or SEQ ID NO: 50 and a first VL comprising the amino acid sequence of SEQ ID NO: 49; and comprises a second VH/VL unit comprising a second VH comprising the amino acid sequence of SEQ ID NO: 13 or SEQ ID NO: 11 and a second VL comprising the amino acid sequence of SEQ ID NO: 14 or SEQ ID NO: 12.

In some embodiments, the multispecific antibody comprises an antigen-binding domain that specifically binds to IL-17 and IL-13 where the antibody comprises a first VH/VL unit comprising a first VH comprising the amino acid sequence of SEQ ID NO: 48 or SEQ ID NO: 50 and a first VL comprising the amino acid sequence of SEQ ID NO: 49; and comprises a second VH/VL unit comprising a second VH comprising the amino acid sequence of SEQ ID NO: 30 and a second VL comprising the amino acid sequence of SEQ ID NO: 29.

In some embodiments, the multispecific antibody comprises an antigen-binding domain that specifically binds to IL-17 and IL-13 wherein the antibody comprises a first VH/VL unit comprising a VH having at least 90%, 91%, 92%, 93%, 94%, 95%, 96%, 97%, 98%, 99%, or 100% sequence identity to the amino acid sequence of SEQ ID NO: 48 or SEQ ID NO: 50 and a first VL having at least 90%, 91%, 92%, 93%, 94%, 95%, 96%, 97%, 98%, 99%, or 100% sequence identity to the amino acid sequence of SEQ ID NO: 49. In certain embodiments, a total of 1 to 10 amino acids have been substituted, inserted and/or deleted in the sequences above. In certain embodiments, substitutions, insertions, or deletions occur in regions outside the HVRs (i.e., in the FRs).

In some embodiments, the multispecific antibody comprises an antigen-binding domain that specifically binds to IL-17 and IL-13 where the antibody comprises a second VH/VL unit comprising a VH having at least 90%, 91%, 92%, 93%, 94%, 95%, 96%, 97%, 98%, 99%, or 100% sequence identity to the amino acid sequence of SEQ ID NO: 13 and a VL having at least 90%, 91%, 92%, 93%, 94%, 95%, 96%, 97%, 98%, 99%, or 100% sequence identity to the amino acid sequence of SEQ ID NO: 14. In some embodiments, the multispecific antibody comprises an antigen-binding domain that specifically binds to IL-17 and IL-13 where the antibody comprises a second VH/VL unit comprising a VH having at least 90%, 91%, 92%, 93%, 94%, 95%, 96%, 97%, 98%, 99%, or 100% sequence identity to the amino acid sequence of SEQ ID NO: 30 and a VL having at least 90%, 91%, 92%, 93%, 94%, 95%, 96%, 97%, 98%, 99%, or 100% sequence identity to the amino acid sequence of SEQ ID NO: 29. In certain embodiments, a total of 1 to 10 amino acids have been substituted, inserted and/or deleted in the sequences above. In certain embodiments, substitutions, insertions, or deletions occur in regions outside the HVRs (i.e., in the FRs).

In some embodiments, the multispecific antibody comprises an antigen-binding domain that specifically binds to IL-17 and IL-13 wherein the antibody comprises a first VH/VL unit comprising a first VH having at least 90%, 91%, 92%, 93%, 94%, 95%, 96%, 97%, 98%, 99%, or 100% sequence identity to the amino acid sequence of SEQ ID NO: 48 or SEQ ID NO: 50 and a first VL having at least 90%, 91%, 92%, 93%, 94%, 95%, 96%, 97%, 98%, 99%, or 100% sequence identity to the amino acid sequence of SEQ ID NO: 49; and a second VH/VL unit comprising a second VH having at least 90%, 91%, 92%, 93%, 94%, 95%, 96%, 97%, 98%, 99%, or 100% sequence identity to the amino acid sequence of SEQ ID NO: 13 and a second VL having at least 90%, 91%, 92%, 93%, 94%, 95%, 96%, 97%, 98%, 99%, or 100% sequence identity to the amino acid sequence of SEQ ID NO: 14. In certain embodiments, a total of 1 to 10 amino acids have been substituted, inserted and/or deleted in the sequences above. In certain embodiments, substitutions, insertions, or deletions occur in regions outside the HVRs (i.e., in the FRs).

In some embodiments, the multispecific antibody comprises an antigen-binding domain that specifically binds to IL-17 and IL-13 wherein the antibody comprises a first VH/VL unit comprising a first VH having at least 90%, 91%, 92%, 93%, 94%, 95%, 96%, 97%, 98%, 99%, or 100% sequence identity to the amino acid sequence of SEQ ID NO: 48 or SEQ ID NO: 50 and a first VL having at least 90%, 91%, 92%, 93%, 94%, 95%, 96%, 97%, 98%, 99%, or 100% sequence identity to the amino acid sequence of SEQ ID NO: 49; and a second VH/VL unit comprising a second VH having at least 90%, 91%, 92%, 93%, 94%, 95%, 96%, 97%, 98%, 99%, or 100% sequence identity to the amino acid sequence of SEQ ID NO: 30 and a second VL having at least 90%, 91%, 92%, 93%, 94%, 95%, 96%, 97%, 98%, 99%, or 100% sequence identity to the amino acid sequence of SEQ ID NO: 29. In certain embodiments, a total of 1 to 10 amino acids have been substituted, inserted and/or deleted in the sequences above. In certain embodiments, substitutions, insertions, or deletions occur in regions outside the HVRs (i.e., in the FRs).

In some embodiments, a multispecific antibody comprises an antigen-binding domain that specifically binds to IL-17 and IL-13 where the antibody comprises a first VH/VL unit comprising at least one, two, three, four, five, or six HVRs selected from (a) HVR-H1 comprising the amino acid sequence of SEQ ID NO: 51; (b) HVR-H2 comprising the amino acid sequence of SEQ ID NO: 52 or SEQ ID NO: 53; (c) HVR-H3 comprising the amino acid sequence of SEQ ID NO: 54; (d) HVR-L1 comprising the amino acid sequence of SEQ ID NO: 55; (e) HVR-L2 comprising the amino acid sequence of SEQ ID NO: 56; and (f) HVR-L3 comprising the amino acid sequence of SEQ ID NO: 57.

In some embodiments, a multispecific antibody comprises an antigen-binding domain that specifically binds to IL-17 and IL-13 where the antibody comprises a second VH/VL unit comprising at least one, two, three, four, five, or six HVRs selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17; (d) HVR-L1 comprising the amino acid sequence of SEQ ID NO: 18; (e) HVR-L2 comprising the amino acid sequence of SEQ ID NO: 19; and (f) HVR-L3 comprising the amino acid sequence of SEQ ID NO: 20. In some embodiments, a multispecific antibody comprises an antigen-binding domain that specifically binds to IL-17 and IL-13 where the antibody comprises a second VH/VL unit comprising at least one, two, three, four, five, or six HVRs selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33; (d) HVR-L1 comprising the amino acid sequence of SEQ ID NO: 34; (e) HVR-L2 comprising the amino acid sequence of SEQ ID NO: 35; and (f) HVR-L3 comprising the amino acid sequence of SEQ ID NO: 36.

In some embodiments, a multispecific antibody comprises an antigen-binding domain that specifically binds to IL-17 and IL-13 where the antibody comprises a first VH/VL unit comprising at least one, two, three, four, five, or six HVRs selected from (a) HVR-H1 comprising the amino acid sequence of SEQ ID NO: 51; (b) HVR-H2 comprising the amino acid sequence of SEQ ID NO: 52 or SEQ ID NO: 53; (c) HVR-H3 comprising the amino acid sequence of SEQ ID NO: 54; (d) HVR-L1 comprising the amino acid sequence of SEQ ID NO: 55; (e) HVR-L2 comprising the amino acid sequence of SEQ ID NO: 56; and (f) HVR-L3 comprising the amino acid sequence of SEQ ID NO: 57; and a second VH/VL unit comprising at least one, two, three, four, five, or six HVRs selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17; (d) HVR-L1 comprising the amino acid sequence of SEQ ID NO: 18; (e) HVR-L2 comprising the amino acid sequence of SEQ ID NO: 19; and (f) HVR-L3 comprising the amino acid sequence of SEQ ID NO: 20.

In some embodiments, a multispecific antibody comprises an antigen-binding domain that specifically binds to IL-17 and IL-13 where the antibody comprises a first VH/VL unit comprising at least one, two, three, four, five, or six HVRs selected from (a) HVR-H1 comprising the amino acid sequence of SEQ ID NO: 51; (b) HVR-H2 comprising the amino acid sequence of SEQ ID NO: 52 or SEQ ID NO: 53; (c) HVR-H3 comprising the amino acid sequence of SEQ ID NO: 54; (d) HVR-L1 comprising the amino acid sequence of SEQ ID NO: 55; (e) HVR-L2 comprising the amino acid sequence of SEQ ID NO: 56; and (f) HVR-L3 comprising the amino acid sequence of SEQ ID NO: 57; and a second VH/VL unit comprising at least one, two, three, four, five, or six HVRs selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33; (d) HVR-L1 comprising the amino acid sequence of SEQ ID NO: 34; (e) HVR-L2 comprising the amino acid sequence of SEQ ID NO: 35; and (f) HVR-L3 comprising the amino acid sequence of SEQ ID NO: 36.

In some embodiments, a multispecific antibody comprises an antigen-binding domain that specifically binds to IL-17 and IL-13 where the antibody comprises a first VH/VL unit comprising at least one, at least two, or all three VH HVR sequences selected from (a) HVR-H1 comprising the amino acid sequence of SEQ ID NO: 51; (b) HVR-H2 comprising the amino acid sequence of SEQ ID NO: 52 or SEQ ID NO: 53; (c) HVR-H3 comprising the amino acid sequence of SEQ ID NO: 54.

In some embodiments, a multispecific antibody comprises an antigen-binding domain that specifically binds to IL-17 and IL-13 where the antibody comprises a second VH/VL unit comprising at least one, at least two, or all three VH HVR sequences selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17. In some embodiments, a multi specific antibody comprises an antigen-binding domain that specifically binds to IL-17 and IL-13 where the antibody comprises a second VH/VL unit comprising at least one, at least two, or all three VH HVR sequences selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33.

In some embodiments, a multispecific antibody comprises an antigen-binding domain that specifically binds to IL-17 and IL-13 where the antibody comprises a first VH/VL unit comprising at least one, at least two, or all three VH HVR sequences selected from (a) HVR-H1 comprising the amino acid sequence of SEQ ID NO: 51; (b) HVR-H2 comprising the amino acid sequence of SEQ ID NO: 52 or SEQ ID NO: 53; (c) HVR-H3 comprising the amino acid sequence of SEQ ID NO: 54; and a second VH/VL unit comprising at least one, at least two, or all three VH HVR sequences selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17. In some embodiments, a multispecific antibody comprises an antigen-binding domain that specifically binds to IL-17 and IL-13 where the antibody comprises a first VH/VL unit comprising at least one, at least two, or all three VH HVR sequences selected from (a) HVR-H1 comprising the amino acid sequence of SEQ ID NO: 51; (b) HVR-H2 comprising the amino acid sequence of SEQ ID NO: 52 or SEQ ID NO: 53; (c) HVR-H3 comprising the amino acid sequence of SEQ ID NO: 54; and a second VH/VL unit comprising at least one, at least two, or all three VH HVR sequences selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33.

In some embodiments, a multispecific antibody comprises an antigen-binding domain that specifically binds to IL-17 and IL-13 where the antibody comprises a first VH/VL unit comprising at least one, at least two, or all three VL HVR sequences selected from (a) HVR-L1 comprising the amino acid sequence of SEQ ID NO: 55; (b) HVR-L2 comprising the amino acid sequence of SEQ ID NO: 56; and (c) HVR-L3 comprising the amino acid sequence of SEQ ID NO: 57.

In some embodiments, a multispecific antibody comprises an antigen-binding domain that specifically binds to IL-17 and IL-13 where the antibody comprises a second VH/VL unit comprising at least one, at least two, or all three VL HVR sequences selected from (a) HVR-L1 comprising the amino acid sequence of SEQ ID NO: 18; (b) HVR-L2 comprising the amino acid sequence of SEQ ID NO: 19; and (c) HVR-L3 comprising the amino acid sequence of SEQ ID NO: 20. In some embodiments, a multispecific antibody comprises an antigen-binding domain that specifically binds to IL-17 and IL-13 where the antibody comprises a second VH/VL unit comprising at least one, at least two, or all three VL HVR sequences selected from (a) HVR-L1 comprising the amino acid sequence of SEQ ID NO: 34; (b) HVR-L2 comprising the amino acid sequence of SEQ ID NO: 35; and (c) HVR-L3 comprising the amino acid sequence of SEQ ID NO: 36.

In some embodiments, a multispecific antibody comprises an antigen-binding domain that specifically binds to IL-17 and IL-13 where the antibody comprises a first VH/VL unit comprising at least one, at least two, or all three VL HVR sequences selected from (a) HVR-L1 comprising the amino acid sequence of SEQ ID NO: 55; (b) HVR-L2 comprising the amino acid sequence of SEQ ID NO: 56; and (c) HVR-L3 comprising the amino acid sequence of SEQ ID NO: 57; and a second VH/VL unit comprising at least one, at least two, or all three VL HVR sequences selected from (a) HVR-L1 comprising the amino acid sequence of SEQ ID NO: 18; (b) HVR-L2 comprising the amino acid sequence of SEQ ID NO: 19; and (c) HVR-L3 comprising the amino acid sequence of SEQ ID NO: 20. In some embodiments, a multispecific antibody comprises an antigen-binding domain that specifically binds to IL-17 and IL-13 where the antibody comprises a first VH/VL unit comprising at least one, at least two, or all three VL HVR sequences selected from (a) HVR-L1 comprising the amino acid sequence of SEQ ID NO: 55; (b) HVR-L2 comprising the amino acid sequence of SEQ ID NO: 56; and (c) HVR-L3 comprising the amino acid sequence of SEQ ID NO: 57; and a second VH/VL unit comprising at least one, at least two, or all three VL HVR sequences selected from (a) HVR-L1 comprising the amino acid sequence of SEQ ID NO: 34; (b) HVR-L2 comprising the amino acid sequence of SEQ ID NO: 35; and (c) HVR-L3 comprising the amino acid sequence of SEQ ID NO: 36.

In some embodiments, a multispecific antibody comprises an antigen-binding domain that specifically binds to IL-17 and IL-13 where the antibody comprises a first VH/VL unit comprising three VH HVR sequences selected from (a) HVR-H1 comprising the amino acid sequence of SEQ ID NO: 51; (b) HVR-H2 comprising the amino acid sequence of SEQ ID NO: 52 or SEQ ID NO: 53; (c) HVR-H3 comprising the amino acid sequence of SEQ ID NO: 54; and three VL HVR sequences selected from (d) HVR-L1 comprising the amino acid sequence of SEQ ID NO: 55; (e) HVR-L2 comprising the amino acid sequence of SEQ ID NO: 56; and (f) HVR-L3 comprising an amino acid sequence selected from SEQ ID NO: 57.

In some embodiments, a multispecific antibody comprises an antigen-binding domain that specifically binds to IL-17 and IL-13 where the antibody comprises a second VH/VL unit comprising three VH HVR sequences selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17; and three VL HVR sequences selected from (a) HVR-L1 comprising the amino acid sequence of SEQ ID NO: 18; (b) HVR-L2 comprising the amino acid sequence of SEQ ID NO: 19; and (c) HVR-L3 comprising the amino acid sequence of SEQ ID NO: 20.

In some embodiments, a multispecific antibody comprises an antigen-binding domain that specifically binds to IL-17 and IL-13 where the antibody comprises a second VH/VL unit comprising three VH HVR sequences selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33; and three VL HVR sequences selected from (a) HVR-L1 comprising the amino acid sequence of SEQ ID NO: 34; (b) HVR-L2 comprising the amino acid sequence of SEQ ID NO: 35; and (c) HVR-L3 comprising the amino acid sequence of SEQ ID NO: 36.

In some embodiments, a multispecific antibody comprises an antigen-binding domain that specifically binds to IL-17 and IL-13 where the antibody comprises a first VH/VL unit comprising three VH HVR sequences selected from (a) HVR-H1 comprising the amino acid sequence of SEQ ID NO: 51; (b) HVR-H2 comprising the amino acid sequence of SEQ ID NO: 52 or SEQ ID NO: 53; (c) HVR-H3 comprising the amino acid sequence of SEQ ID NO: 54; and three VL HVR sequences selected from (d) HVR-L1 comprising the amino acid sequence of SEQ ID NO: 55; (e) HVR-L2 comprising the amino acid sequence of SEQ ID NO: 56; and (f) HVR-L3 comprising an amino acid sequence selected from SEQ ID NO: 57; and a second VH/VL unit comprising three VH HVR sequences selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17; and three VL HVR sequences selected from (a) HVR-L1 comprising the amino acid sequence of SEQ ID NO: 18; (b) HVR-L2 comprising the amino acid sequence of SEQ ID NO: 19; and (c) HVR-L3 comprising the amino acid sequence of SEQ ID NO: 20.

In some embodiments, a multispecific antibody comprises an antigen-binding domain that specifically binds to IL-17 and IL-13 where the antibody comprises a first VH/VL unit comprising three VH HVR sequences selected from (a) HVR-H1 comprising the amino acid sequence of SEQ ID NO: 51; (b) HVR-H2 comprising the amino acid sequence of SEQ ID NO: 52 or SEQ ID NO: 53; (c) HVR-H3 comprising the amino acid sequence of SEQ ID NO: 54; and three VL HVR sequences selected from (d) HVR-L1 comprising the amino acid sequence of SEQ ID NO: 55; (e) HVR-L2 comprising the amino acid sequence of SEQ ID NO: 56; and (f) HVR-L3 comprising an amino acid sequence selected from SEQ ID NO: 57; and a second VH/VL unit comprising three VH HVR sequences selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33; and three VL HVR sequences selected from (a) HVR-L1 comprising the amino acid sequence of SEQ ID NO: 34; (b) HVR-L2 comprising the amino acid sequence of SEQ ID NO: 35; and (c) HVR-L3 comprising the amino acid sequence of SEQ ID NO: 36.

In various embodiments, a multispecific antibody comprises a first hemimer comprising a first VH/VL unit that binds IL-17, wherein the first hemimer comprises a knob mutation in the heavy chain constant region, and a second hemimer comprising a second VH/VL unit that binds IL-13, wherein the second hemimer comprises a hole mutation in the heavy chain constant region. In various embodiments, a multispecific antibody comprises a first hemimer comprising a first VH/VL unit that binds IL-17, wherein the first hemimer comprises a hole mutation in the heavy chain constant region, and a second hemimer comprising a second VH/VL unit that binds IL-13, wherein the second hemimer comprises a knob mutation in the heavy chain constant region. In some embodiments, a heavy chain constant region comprising a hole mutation has the sequence shown in SEQ ID NO: 68 (IgG1) or SEQ ID NO: 70 (IgG4). In some embodiments, a heavy chain constant region comprising a knob mutation has the sequence shown in SEQ ID NO: 67 (IgG1) or SEQ ID NO: 69 (IgG4). In some embodiments, a multispecific antibody comprises a first hemimer comprising a first heavy chain having the sequence of SEQ ID NO: 74 or 75 and a first light chain having the sequence of SEQ ID NO: 76, and a second hemimer comprising a second heavy chain having the sequence of SEQ ID NO: 21 or 23 and a second light chain having the sequence of SEQ ID NO: 22 or 24. In some embodiments, a multispecific antibody comprises a first hemimer comprising a first heavy chain having the sequence of SEQ ID NO: 74 or 75 and a first light chain having the sequence of SEQ ID NO: 76, and a second hemimer comprising a second heavy chain having the sequence of SEQ ID NO: 21 and a second light chain having the sequence of SEQ ID NO: 22.

In some embodiments, an anti-IL-13/IL-17 multispecific antibody according to any of the above embodiments is a monoclonal antibody, including a chimeric, humanized or human antibody. In some embodiments, an anti-IL-13/IL-17 multispecific antibody is an antibody fragment, e.g., a Fv, Fab, Fab′, scFv, diabody, or F(ab′)₂ fragment. In some embodiments, the antibody is a full length antibody, e.g., an intact IgG1 or IgG4 antibody or other antibody class or isotype as defined herein.

In some embodiments, an anti-IL-13/IL-17 multispecific antibody according to any of the above embodiments may incorporate any of the features, singly or in combination, as described in Sections 1-7 below.

### Multispecific Antibodies Comprising 39F12 or 39F12A

In some embodiments, the multispecific antibody comprises an antigen-binding domain that specifically binds to IL-17 and IL-13 where the antibody comprises a first VH/VL unit comprising a VH (heavy chain variable domain) comprising the amino acid sequence of SEQ ID NO: 58. In some embodiments, the multispecific antibody comprises an antigen-binding domain that specifically binds to IL-17 and IL-13 where the antibody comprises a first VH/VL unit comprising a VL (light chain variable domain) comprising an amino acid sequence selected from SEQ ID NOs: 59 and 60.

In some embodiments, the multispecific antibody comprises an antigen-binding domain that specifically binds to IL-17 and IL-13 where the antibody comprises a first VH/VL unit comprising a VH comprising the amino acid sequence of SEQ ID NO: 58 and a VL comprising the amino acid sequence of SEQ ID NO: 59. In some embodiments, the multispecific antibody comprises an antigen-binding domain that specifically binds to IL-17 and IL-13 where the antibody comprises a first VH/VL unit comprising a VH comprising the amino acid sequence of SEQ ID NO: 60 and a VL comprising the amino acid sequence of SEQ ID NO: 59.

In some embodiments, the multispecific antibody comprises an antigen-binding domain that specifically binds to IL-17 and IL-13 where the antibody comprises a first VH/VL unit that competes for binding to IL-17 with an antibody comprising a VH comprising the amino acid sequence of SEQ ID NO: 58 and a VL comprising the amino acid sequence of SEQ ID NO: 60.

In some embodiments, the multispecific antibody comprises an antigen-binding domain that specifically binds to IL-17 and IL-13 where the antibody comprises a second VH/VL unit comprising a VH (heavy chain variable domain) comprising the amino acid sequence of SEQ ID NO: 13 or SEQ ID NO: 11. In some embodiments, the multispecific antibody comprises an antigen-binding domain that specifically binds to IL-17 and IL-13 where the antibody comprises a second VH/VL unit comprising a VL (light chain variable domain) comprising the amino acid sequence of SEQ ID NO: 14 or SEQ ID NO: 12. In some embodiments, the multispecific antibody comprises an antigen-binding domain that specifically binds to IL-17 and IL-13 where the antibody comprises a second VH/VL unit comprising a VH comprising the amino acid sequence of SEQ ID NO: 13 or SEQ ID NO: 11 and a VL comprising the amino acid sequence of SEQ ID NO: 14 or SEQ ID NO: 12. In some embodiments, the multispecific antibody comprises an antigen-binding domain that specifically binds to IL-17 and IL-13 where the antibody comprises a second VH/VL unit that competes for binding to IL-13 with an antibody comprising a VH comprising the amino acid sequence of SEQ ID NO: 13 and a VL comprising the amino acid sequence of SEQ ID NO: 14. In some embodiments, the multispecific antibody comprises an antigen-binding domain that specifically binds to IL-17 and IL-13 where the antibody comprises a second VH/VL unit that binds an epitope of IL-13 consisting of amino acids 82 to 89 of SEQ ID NO: 1. In some embodiments, the multispecific antibody comprises an antigen-binding domain that specifically binds to IL-17 and IL-13 where the antibody comprises a second VH/VL unit that binds an epitope of IL-13 consisting of amino acids 77 to 89 of SEQ ID NO: 1.

In some embodiments, the multispecific antibody comprises an antigen-binding domain that specifically binds to IL-17 and IL-13 where the antibody comprises a second VH/VL unit comprising a VH (heavy chain variable domain) comprising the amino acid sequence of SEQ ID NO: 30. In some embodiments, the multispecific antibody comprises an antigen-binding domain that specifically binds to IL-17 and IL-13 where the antibody comprises a second VH/VL unit comprising a VL (light chain variable domain) comprising the amino acid sequence of SEQ ID NO: 29. In some embodiments, the multispecific antibody comprises an antigen-binding domain that specifically binds to IL-17 and IL-13 where the antibody comprises a second VH/VL unit comprising a VH comprising the amino acid sequence of SEQ ID NO: 30 and a VL comprising the amino acid sequence of SEQ ID NO: 29. In some embodiments, the multispecific antibody comprises an antigen-binding domain that specifically binds to IL-17 and IL-13 where the antibody comprises a second VH/VL unit that competes for binding to IL-13 with an antibody comprising a VH comprising the amino acid sequence of SEQ ID NO: 30 and a VL comprising the amino acid sequence of SEQ ID NO: 29.

In some embodiments, the multispecific antibody comprises an antigen-binding domain that specifically binds to IL-17 and IL-13 where the antibody comprises a first VH/VL unit comprising a first VH comprising the amino acid sequence of SEQ ID NO: 58 and a first VL comprising the amino acid sequence of SEQ ID NO: 59 or SEQ ID NO: 60; and comprises a second VH/VL unit comprising a second VH comprising the amino acid sequence of SEQ ID NO: 13 or SEQ ID NO: 11 and a second VL comprising the amino acid sequence of SEQ ID NO: 14 or SEQ ID NO: 12.

In some embodiments, the multispecific antibody comprises an antigen-binding domain that specifically binds to IL-17 and IL-13 where the antibody comprises a first VH/VL unit comprising a first VH comprising the amino acid sequence of SEQ ID NO: 58 and a first VL comprising the amino acid sequence of SEQ ID NO: 59 or SEQ ID NO: 60; and comprises a second VH/VL unit comprising a second VH comprising the amino acid sequence of SEQ ID NO: 30 and a second VL comprising the amino acid sequence of SEQ ID NO: 29.

In some embodiments, the multispecific antibody comprises an antigen-binding domain that specifically binds to IL-17 and IL-13 wherein the antibody comprises a first VH/VL unit comprising a VH having at least 90%, 91%, 92%, 93%, 94%, 95%, 96%, 97%, 98%, 99%, or 100% sequence identity to the amino acid sequence of SEQ ID NO: 58 and a first VL having at least 90%, 91%, 92%, 93%, 94%, 95%, 96%, 97%, 98%, 99%, or 100% sequence identity to the amino acid sequence of SEQ ID NO: 59 or SEQ ID NO: 60. In certain embodiments, a total of 1 to 10 amino acids have been substituted, inserted and/or deleted in the sequences above. In certain embodiments, substitutions, insertions, or deletions occur in regions outside the HVRs (i.e., in the FRs).

In some embodiments, the multispecific antibody comprises an antigen-binding domain that specifically binds to IL-17 and IL-13 where the antibody comprises a second VH/VL unit comprising a VH having at least 90%, 91%, 92%, 93%, 94%, 95%, 96%, 97%, 98%, 99%, or 100% sequence identity to the amino acid sequence of SEQ ID NO: 13 and a VL having at least 90%, 91%, 92%, 93%, 94%, 95%, 96%, 97%, 98%, 99%, or 100% sequence identity to the amino acid sequence of SEQ ID NO: 14. In some embodiments, the multispecific antibody comprises an antigen-binding domain that specifically binds to IL-17 and IL-13 where the antibody comprises a second VH/VL unit comprising a VH having at least 90%, 91%, 92%, 93%, 94%, 95%, 96%, 97%, 98%, 99%, or 100% sequence identity to the amino acid sequence of SEQ ID NO: 30 and a VL having at least 90%, 91%, 92%, 93%, 94%, 95%, 96%, 97%, 98%, 99%, or 100% sequence identity to the amino acid sequence of SEQ ID NO: 29. In certain embodiments, a total of 1 to 10 amino acids have been substituted, inserted and/or deleted in the sequences above. In certain embodiments, substitutions, insertions, or deletions occur in regions outside the HVRs (i.e., in the FRs).

In some embodiments, the multispecific antibody comprises an antigen-binding domain that specifically binds to IL-17 and IL-13 wherein the antibody comprises a first VH/VL unit comprising a first VH having at least 90%, 91%, 92%, 93%, 94%, 95%, 96%, 97%, 98%, 99%, or 100% sequence identity to the amino acid sequence of SEQ ID NO: 58 and a first VL having at least 90%, 91%, 92%, 93%, 94%, 95%, 96%, 97%, 98%, 99%, or 100% sequence identity to the amino acid sequence of SEQ ID NO: 59 or SEQ ID NO: 60; and a second VH/VL unit comprising a second VH having at least 90%, 91%, 92%, 93%, 94%, 95%, 96%, 97%, 98%, 99%, or 100% sequence identity to the amino acid sequence of SEQ ID NO: 13 and a second VL having at least 90%, 91%, 92%, 93%, 94%, 95%, 96%, 97%, 98%, 99%, or 100% sequence identity to the amino acid sequence of SEQ ID NO: 14. In certain embodiments, a total of 1 to 10 amino acids have been substituted, inserted and/or deleted in the sequences above. In certain embodiments, substitutions, insertions, or deletions occur in regions outside the HVRs (i.e., in the FRs).

In some embodiments, the multispecific antibody comprises an antigen-binding domain that specifically binds to IL-17 and IL-13 wherein the antibody comprises a first VH/VL unit comprising a first VH having at least 90%, 91%, 92%, 93%, 94%, 95%, 96%, 97%, 98%, 99%, or 100% sequence identity to the amino acid sequence of SEQ ID NO: 58 and a first VL having at least 90%, 91%, 92%, 93%, 94%, 95%, 96%, 97%, 98%, 99%, or 100% sequence identity to the amino acid sequence of SEQ ID NO: 59 or SEQ ID NO: 60; and a second VH/VL unit comprising a second VH having at least 90%, 91%, 92%, 93%, 94%, 95%, 96%, 97%, 98%, 99%, or 100% sequence identity to the amino acid sequence of SEQ ID NO: 30 and a second VL having at least 90%, 91%, 92%, 93%, 94%, 95%, 96%, 97%, 98%, 99%, or 100% sequence identity to the amino acid sequence of SEQ ID NO: 29. In certain embodiments, a total of 1 to 10 amino acids have been substituted, inserted and/or deleted in the sequences above. In certain embodiments, substitutions, insertions, or deletions occur in regions outside the HVRs (i.e., in the FRs).

In some embodiments, a multispecific antibody comprises an antigen-binding domain that specifically binds to IL-17 and IL-13 where the antibody comprises a first VH/VL unit comprising at least one, two, three, four, five, or six HVRs selected from (a) HVR-H1 comprising the amino acid sequence of SEQ ID NO: 61; (b) HVR-H2 comprising the amino acid sequence of SEQ ID NO: 62; (c) HVR-H3 comprising the amino acid sequence of SEQ ID NO: 63; (d) HVR-L1 comprising the amino acid sequence of SEQ ID NO: 64; (e) HVR-L2 comprising the amino acid sequence of SEQ ID NO: 65; and (f) HVR-L3 comprising the amino acid sequence of SEQ ID NO: 66.

In some embodiments, a multispecific antibody comprises an antigen-binding domain that specifically binds to IL-17 and IL-13 where the antibody comprises a second VH/VL unit comprising at least one, two, three, four, five, or six HVRs selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17; (d) HVR-L1 comprising the amino acid sequence of SEQ ID NO: 18; (e) HVR-L2 comprising the amino acid sequence of SEQ ID NO: 19; and (f) HVR-L3 comprising the amino acid sequence of SEQ ID NO: 20. In some embodiments, a multispecific antibody comprises an antigen-binding domain that specifically binds to IL-17 and IL-13 where the antibody comprises a second VH/VL unit comprising at least one, two, three, four, five, or six HVRs selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33; (d) HVR-L1 comprising the amino acid sequence of SEQ ID NO: 34; (e) HVR-L2 comprising the amino acid sequence of SEQ ID NO: 35; and (f) HVR-L3 comprising the amino acid sequence of SEQ ID NO: 36.

In some embodiments, a multispecific antibody comprises an antigen-binding domain that specifically binds to IL-17 and IL-13 where the antibody comprises a first VH/VL unit comprising at least one, two, three, four, five, or six HVRs selected from (a) HVR-H1 comprising the amino acid sequence of SEQ ID NO: 61; (b) HVR-H2 comprising the amino acid sequence of SEQ ID NO: 62; (c) HVR-H3 comprising the amino acid sequence of SEQ ID NO: 63; (d) HVR-L1 comprising the amino acid sequence of SEQ ID NO: 64; (e) HVR-L2 comprising the amino acid sequence of SEQ ID NO: 65; and (f) HVR-L3 comprising the amino acid sequence of SEQ ID NO: 66; and a second VH/VL unit comprising at least one, two, three, four, five, or six HVRs selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17; (d) HVR-L1 comprising the amino acid sequence of SEQ ID NO: 18; (e) HVR-L2 comprising the amino acid sequence of SEQ ID NO: 19; and (f) HVR-L3 comprising the amino acid sequence of SEQ ID NO: 20.

In some embodiments, a multispecific antibody comprises an antigen-binding domain that specifically binds to IL-17 and IL-13 where the antibody comprises a first VH/VL unit comprising at least one, two, three, four, five, or six HVRs selected from (a) HVR-H1 comprising the amino acid sequence of SEQ ID NO: 61; (b) HVR-H2 comprising the amino acid sequence of SEQ ID NO: 62; (c) HVR-H3 comprising the amino acid sequence of SEQ ID NO: 63; (d) HVR-L1 comprising the amino acid sequence of SEQ ID NO: 64; (e) HVR-L2 comprising the amino acid sequence of SEQ ID NO: 65; and (f) HVR-L3 comprising the amino acid sequence of SEQ ID NO: 66; and a second VH/VL unit comprising at least one, two, three, four, five, or six HVRs selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33; (d) HVR-L1 comprising the amino acid sequence of SEQ ID NO: 34; (e) HVR-L2 comprising the amino acid sequence of SEQ ID NO: 35; and (f) HVR-L3 comprising the amino acid sequence of SEQ ID NO: 36.

In some embodiments, a multispecific antibody comprises an antigen-binding domain that specifically binds to IL-17 and IL-13 where the antibody comprises a first VH/VL unit comprising at least one, at least two, or all three VH HVR sequences selected from (a) HVR-H1 comprising the amino acid sequence of SEQ ID NO: 61; (b) HVR-H2 comprising the amino acid sequence of SEQ ID NO: 62; (c) HVR-H3 comprising the amino acid sequence of SEQ ID NO: 63.

In some embodiments, a multispecific antibody comprises an antigen-binding domain that specifically binds to IL-17 and IL-13 where the antibody comprises a second VH/VL unit comprising at least one, at least two, or all three VH HVR sequences selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17. In some embodiments, a multispecific antibody comprises an antigen-binding domain that specifically binds to IL-17 and IL-13 where the antibody comprises a second VH/VL unit comprising at least one, at least two, or all three VH HVR sequences selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33.

In some embodiments, a multispecific antibody comprises an antigen-binding domain that specifically binds to IL-17 and IL-13 where the antibody comprises a first VH/VL unit comprising at least one, at least two, or all three VH HVR sequences selected from (a) HVR-H1 comprising the amino acid sequence of SEQ ID NO: 61; (b) HVR-H2 comprising the amino acid sequence of SEQ ID NO: 62; (c) HVR-H3 comprising the amino acid sequence of SEQ ID NO: 63; and a second VH/VL unit comprising at least one, at least two, or all three VH HVR sequences selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17. In some embodiments, a multispecific antibody comprises an antigen-binding domain that specifically binds to IL-17 and IL-13 where the antibody comprises a first VH/VL unit comprising at least one, at least two, or all three VH HVR sequences selected from (a) HVR-H1 comprising the amino acid sequence of SEQ ID NO: 61; (b) HVR-H2 comprising the amino acid sequence of SEQ ID NO: 62; (c) HVR-H3 comprising the amino acid sequence of SEQ ID NO: 63; and a second VH/VL unit comprising at least one, at least two, or all three VH HVR sequences selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33.

In some embodiments, a multispecific antibody comprises an antigen-binding domain that specifically binds to IL-17 and IL-13 where the antibody comprises a first VH/VL unit comprising at least one, at least two, or all three VL HVR sequences selected from (a) HVR-L1 comprising the amino acid sequence of SEQ ID NO: 64; (b) HVR-L2 comprising the amino acid sequence of SEQ ID NO: 65; and (c) HVR-L3 comprising the amino acid sequence of SEQ ID NO: 66.

In some embodiments, a multispecific antibody comprises an antigen-binding domain that specifically binds to IL-17 and IL-13 where the antibody comprises a second VH/VL unit comprising at least one, at least two, or all three VL HVR sequences selected from (a) HVR-L1 comprising the amino acid sequence of SEQ ID NO: 18; (b) HVR-L2 comprising the amino acid sequence of SEQ ID NO: 19; and (c) HVR-L3 comprising the amino acid sequence of SEQ ID NO: 20. In some embodiments, a multispecific antibody comprises an antigen-binding domain that specifically binds to IL-17 and IL-13 where the antibody comprises a second VH/VL unit comprising at least one, at least two, or all three VL HVR sequences selected from (a) HVR-L1 comprising the amino acid sequence of SEQ ID NO: 34; (b) HVR-L2 comprising the amino acid sequence of SEQ ID NO: 35; and (c) HVR-L3 comprising the amino acid sequence of SEQ ID NO: 36.

In some embodiments, a multispecific antibody comprises an antigen-binding domain that specifically binds to IL-17 and IL-13 where the antibody comprises a first VH/VL unit comprising at least one, at least two, or all three VL HVR sequences selected from (a) HVR-L1 comprising the amino acid sequence of SEQ ID NO: 64; (b) HVR-L2 comprising the amino acid sequence of SEQ ID NO: 65; and (c) HVR-L3 comprising the amino acid sequence of SEQ ID NO: 66; and a second VH/VL unit comprising at least one, at least two, or all three VL HVR sequences selected from (a) HVR-L1 comprising the amino acid sequence of SEQ ID NO: 18; (b) HVR-L2 comprising the amino acid sequence of SEQ ID NO: 19; and (c) HVR-L3 comprising the amino acid sequence of SEQ ID NO: 20. In some embodiments, a multispecific antibody comprises an antigen-binding domain that specifically binds to IL-17 and IL-13 where the antibody comprises a first VH/VL unit comprising at least one, at least two, or all three VL HVR sequences selected from (a) HVR-L1 comprising the amino acid sequence of SEQ ID NO: 64; (b) HVR-L2 comprising the amino acid sequence of SEQ ID NO: 65; and (c) HVR-L3 comprising the amino acid sequence of SEQ ID NO: 66; and a second VH/VL unit comprising at least one, at least two, or all three VL HVR sequences selected from (a) HVR-L1 comprising the amino acid sequence of SEQ ID NO: 34; (b) HVR-L2 comprising the amino acid sequence of SEQ ID NO: 35; and (c) HVR-L3 comprising the amino acid sequence of SEQ ID NO: 36.

In some embodiments, a multispecific antibody comprises an antigen-binding domain that specifically binds to IL-17 and IL-13 where the antibody comprises a first VH/VL unit comprising three VH HVR sequences selected from (a) HVR-H1 comprising the amino acid sequence of SEQ ID NO: 61; (b) HVR-H2 comprising the amino acid sequence of SEQ ID NO: 62; (c) HVR-H3 comprising the amino acid sequence of SEQ ID NO: 63; and three VL HVR sequences selected from (d) HVR-L1 comprising the amino acid sequence of SEQ ID NO: 64; (e) HVR-L2 comprising the amino acid sequence of SEQ ID NO: 65; and (f) HVR-L3 comprising an amino acid sequence selected from SEQ ID NO: 66.

In some embodiments, a multispecific antibody comprises an antigen-binding domain that specifically binds to IL-17 and IL-13 where the antibody comprises a second VH/VL unit comprising three VH HVR sequences selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17; and three VL HVR sequences selected from (a) HVR-L1 comprising the amino acid sequence of SEQ ID NO: 18; (b) HVR-L2 comprising the amino acid sequence of SEQ ID NO: 19; and (c) HVR-L3 comprising the amino acid sequence of SEQ ID NO: 20.

In some embodiments, a multispecific antibody comprises an antigen-binding domain that specifically binds to IL-17 and IL-13 where the antibody comprises a second VH/VL unit comprising three VH HVR sequences selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33; and three VL HVR sequences selected from (a) HVR-L1 comprising the amino acid sequence of SEQ ID NO: 34; (b) HVR-L2 comprising the amino acid sequence of SEQ ID NO: 35; and (c) HVR-L3 comprising the amino acid sequence of SEQ ID NO: 36.

In some embodiments, a multispecific antibody comprises an antigen-binding domain that specifically binds to IL-17 and IL-13 where the antibody comprises a first VH/VL unit comprising three VH HVR sequences selected from (a) HVR-H1 comprising the amino acid sequence of SEQ ID NO: 61; (b) HVR-H2 comprising the amino acid sequence of SEQ ID NO: 62; (c) HVR-H3 comprising the amino acid sequence of SEQ ID NO: 63; and three VL HVR sequences selected from (d) HVR-L1 comprising the amino acid sequence of SEQ ID NO: 64; (e) HVR-L2 comprising the amino acid sequence of SEQ ID NO: 65; and (f) HVR-L3 comprising an amino acid sequence selected from SEQ ID NO: 66; and a second VH/VL unit comprising three VH HVR sequences selected from (a) HVR-H1 comprising the amino acid sequence of SEQ ID NO: 15; (b) HVR-H2 comprising the amino acid sequence of SEQ ID NO: 16; (c) HVR-H3 comprising the amino acid sequence of SEQ ID NO: 17; and three VL HVR sequences selected from (a) HVR-L1 comprising the amino acid sequence of SEQ ID NO: 18; (b) HVR-L2 comprising the amino acid sequence of SEQ ID NO: 19; and (c) HVR-L3 comprising the amino acid sequence of SEQ ID NO: 20.

In some embodiments, a multispecific antibody comprises an antigen-binding domain that specifically binds to IL-17 and IL-13 where the antibody comprises a first VH/VL unit comprising three VH HVR sequences selected from (a) HVR-H1 comprising the amino acid sequence of SEQ ID NO: 61; (b) HVR-H2 comprising the amino acid sequence of SEQ ID NO: 62; (c) HVR-H3 comprising the amino acid sequence of SEQ ID NO: 63; and three VL HVR sequences selected from (d) HVR-L1 comprising the amino acid sequence of SEQ ID NO: 64; (e) HVR-L2 comprising the amino acid sequence of SEQ ID NO: 65; and (f) HVR-L3 comprising an amino acid sequence selected from SEQ ID NO: 66; and a second VH/VL unit comprising three VH HVR sequences selected from (a) HVR-H1 comprising the amino acid sequence of SEQ ID NO: 31; (b) HVR-H2 comprising the amino acid sequence of SEQ ID NO: 32; (c) HVR-H3 comprising the amino acid sequence of SEQ ID NO: 33; and three VL HVR sequences selected from (a) HVR-L1 comprising the amino acid sequence of SEQ ID NO: 34; (b) HVR-L2 comprising the amino acid sequence of SEQ ID NO: 35; and (c) HVR-L3 comprising the amino acid sequence of SEQ ID NO: 36.

In various embodiments, a multispecific antibody comprises a first hemimer comprising a first VH/VL unit that binds IL-17, wherein the first hemimer comprises a knob mutation in the heavy chain constant region, and a second hemimer comprising a second VH/VL unit that binds IL-13, wherein the second hemimer comprises a hole mutation in the heavy chain constant region. In various embodiments, a multispecific antibody comprises a first hemimer comprising a first VH/VL unit that binds IL-17, wherein the first hemimer comprises a hole mutation in the heavy chain constant region, and a second hemimer comprising a second VH/VL unit that binds IL-13, wherein the second hemimer comprises a knob mutation in the heavy chain constant region. In some embodiments, a heavy chain constant region comprising a hole mutation has the sequence shown in SEQ ID NO: 68 (IgG1) or SEQ ID NO: 70 (IgG4). In some embodiments, a heavy chain constant region comprising a knob mutation has the sequence shown in SEQ ID NO: 67 (IgG1) or SEQ ID NO: 69 (IgG4). In some embodiments, a multispecific antibody comprises a first hemimer comprising a first heavy chain having the sequence of SEQ ID NO: 77 and a first light chain having the sequence of SEQ ID NO: 78 or 79, and a second hemimer comprising a second heavy chain having the sequence of SEQ ID NO: 21 or 23 and a second light chain having the sequence of SEQ ID NO: 22 or 24. In some embodiments, a multispecific antibody comprises a first hemimer comprising a first heavy chain having the sequence of SEQ ID NO: 77 and a first light chain having the sequence of SEQ ID NO: 78 or 79 and a second hemimer comprising a second heavy chain having the sequence of SEQ ID NO: 21 and a second light chain having the sequence of SEQ ID NO: 22.

In some embodiments, an anti-IL-13/IL-17 multispecific antibody according to any of the above embodiments is a monoclonal antibody, including a chimeric, humanized or human antibody. In some embodiments, an anti-IL-13/IL-17 multispecific antibody is an antibody fragment, e.g., a Fv, Fab, Fab′, scFv, diabody, or F(ab′)₂ fragment. In some embodiments, the antibody is a full length antibody, e.g., an intact IgG1 or IgG4 antibody or other antibody class or isotype as defined herein.

In some embodiments, an anti-IL-13/IL-17 multispecific antibody according to any of the above embodiments may incorporate any of the features, singly or in combination, as described in Sections 1-7 below.

### 1. Antibody Affinity

In certain embodiments, an antibody provided herein has a dissociation constant (Kd) for an antigen of ≦1 μM, ≦100 nM, ≦10 nM, ≦1 nM, ≦0.1 nM, ≦0.01 nM, or ≦0.001 nM (e.g. 10⁻⁸ M or less, e.g. from 10⁻⁸ M to 10⁻¹³ M, e.g., from 10⁻⁹ M to 10⁻¹³ M).

In some embodiments, Kd is measured by a radiolabeled antigen binding assay (RIA). In some embodiments, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to some embodiments, Kd is measured using a BIACORE® surface plasmon resonance assay. For example, an assay using a BIACORE®-2000 or a BIACORE ®-3000 (BIAcore, Inc., Piscataway, N.J.) is performed at 25° C. with immobilized antigen CM5 chips at ˜10 response units (RU). In some embodiments, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (∥0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensograms. The equilibrium dissociation constant (Kd) is calculated as the ratio k_{off}/k_{on.} See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### 3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies described herein may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for IL-17 and the other is for IL-13. In certain embodiments, one of the binding specificities is for IL-17A homodimer, IL-17F homodimer, and IL-17AF heterodimer, and the other is for IL-13. Bispecific antibodies may also be used to localize cytotoxic agents to cells. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168; U.S. Publication No. 2011/0287009). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers or coiled coils to produce bispecific antibodies (see, e.g., Kostelny et al., J. Immunol., 148(5):1547-1553 (1992) and WO2011/034605); using a furin cleavable tether between a C_{L} domain and a V_{H} domain in a single VH/VL unit (see, e.g., WO2013/119966 and WO2013/055958); using “diabody” technology for making bispecific antibody fragments (see, e.g., Hollinger et al., Proc. Natl. Acad. Sci. USA, 90:6444-6448 (1993)); using immunoglobulin domain crossover for making bispecific antibodies (see, e.g., WO2009/080251); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds, for example, to IL-17 as well as another, different antigen, such as IL-13 (see, US 2008/0069820, for example). The DAF bispecific antibody format eliminates the problem of chain mispairing, a problem often encountered in bispecific antibodies, and yet maintains the natural antibody format.

Knobs into Holes

The use of knobs into holes as a method of producing multispecific antibodies is described, e.g., in U.S. Pat. No. 5,731,168, WO2009/089004, US2009/0182127, US2011/0287009, Marvin and Zhu, Acta Pharmacol. Sin. (2005) 26(6):649-658, and Kontermann (2005) Acta Pharmacol. Sin., 26:1-9. A brief nonlimiting discussion is provided below.

A “protuberance” refers to at least one amino acid side chain which projects from the interface of a first polypeptide and is therefore positionable in a compensatory cavity in the adjacent interface (i.e. the interface of a second polypeptide) so as to stabilize the heteromultimer, and thereby favor heteromultimer formation over homomultimer formation, for example. The protuberance may exist in the original interface or may be introduced synthetically (e.g. by altering nucleic acid encoding the interface). In some embodiments, nucleic acid encoding the interface of the first polypeptide is altered to encode the protuberance. To achieve this, the nucleic acid encoding at least one “original” amino acid residue in the interface of the first polypeptide is replaced with nucleic acid encoding at least one “import” amino acid residue which has a larger side chain volume than the original amino acid residue. It will be appreciated that there can be more than one original and corresponding import residue. The side chain volumes of the various amino residues are shown, for example, in Table 1 of US2011/0287009 or Table 1 of U.S. Pat. No. 7,642,228.

In some embodiments, import residues for the formation of a protuberance are naturally occurring amino acid residues selected from arginine (R), phenylalanine (F), tyrosine (Y) and tryptophan (W). In some embodiments, an import residue is tryptophan or tyrosine. In some embodiment, the original residue for the formation of the protuberance has a small side chain volume, such as alanine, asparagine, aspartic acid, glycine, serine, threonine or valine. See e.g., U.S. Pat. No. 7,642,228.

A “cavity” refers to at least one amino acid side chain which is recessed from the interface of a second polypeptide and therefore accommodates a corresponding protuberance on the adjacent interface of a first polypeptide. The cavity may exist in the original interface or may be introduced synthetically (e.g. by altering nucleic acid encoding the interface). In some embodiments, nucleic acid encoding the interface of the second polypeptide is altered to encode the cavity. To achieve this, the nucleic acid encoding at least one “original” amino acid residue in the interface of the second polypeptide is replaced with DNA encoding at least one “import” amino acid residue which has a smaller side chain volume than the original amino acid residue. It will be appreciated that there can be more than one original and corresponding import residue. In some embodiments, import residues for the formation of a cavity are naturally occurring amino acid residues selected from alanine (A), serine (S), threonine (T) and valine (V). In some embodiments, an import residue is serine, alanine or threonine. In some embodiments, the original residue for the formation of the cavity has a large side chain volume, such as tyrosine, arginine, phenylalanine or tryptophan.

The protuberance is “positionable” in the cavity which means that the spatial location of the protuberance and cavity on the interface of a first polypeptide and second polypeptide respectively and the sizes of the protuberance and cavity are such that the protuberance can be located in the cavity without significantly perturbing the normal association of the first and second polypeptides at the interface. Since protuberances such as Tyr, Phe and Trp do not typically extend perpendicularly from the axis of the interface and have preferred conformations, the alignment of a protuberance with a corresponding cavity may, in some instances, rely on modeling the protuberance/cavity pair based upon a three-dimensional structure such as that obtained by X-ray crystallography or nuclear magnetic resonance (NMR). This can be achieved using widely accepted techniques in the art.

In some embodiments, a knob mutation in an IgG1 constant region is T366W. In some embodiments, a hole mutation in an IgG1 constant region comprises one or more mutations selected from T366S, L368A and Y407V. In some embodiments, a hole mutation in an IgG1 constant region comprises T366S, L368A and Y407V. SEQ ID NO: 67 shows an exemplary IgG1 constant region with a knob mutation and SEQ ID NO: 68 shows an exemplary IgG1 constant region with a hole mutation.

In some embodiments, a knob mutation in an IgG4 constant region is T366W. In some embodiments, a hole mutation in an IgG4 constant region comprises one or more mutations selected from T366S, L368A, and Y407V. In some embodiments, a hole mutation in an IgG4 constant region comprises T366S, L368A, and Y407V. SEQ ID NO: 69 shows an exemplary IgG4 constant region with a knob mutation and SEQ ID NO: 70 shows an exemplary IgG4 constant region with a hole mutation. See e.g., U.S. Pat. No. 7,642,228.

### 7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “conservative substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Conservative |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments, amino acid substitutions can be introduced to alter or eliminate one or more post-translational modifications of the immunoglobulin molecule or to improve antibody production yield. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody provided herein may be made in order to create antibody variants with certain improved properties.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose can be determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, and antibody constant region, such as a heavy chain constant region, comprises a knob mutation and/or a hole mutation to facilitate formation of a multispecific antibody. Nonlimiting exemplary knob mutations and hole mutations, and knob-into-hole technology generally, are described, for example, in U.S. Pat. No. 5,731,168, WO2009/089004, US2009/0182127, US2011/0287009, Marvin and Zhu, Acta Pharmacol. Sin. (2005) 26(6):649-658, and Kontermann (2005) Acta Pharmacol. Sin., 26:1-9. Certain nonlimiting exemplary knob mutations and hole mutations are discussed herein.

In certain embodiments, an antibody variant that possesses some but not all effector functions is provided, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, e.g., Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 (D) and 297 (N) to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

In some embodiments, an antibody constant region comprises more than one of the mutations discussed herein (for example, a knob and/or hole mutation and/or a mutation that increases stability and/or a mutation that decreases ADCC, etc.).

### Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In some embodiments, conjugates of an antibody and nonproteinaceous moiety that may be selectively heated by exposure to radiation are provided. In some embodiments, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### Recombinant Methods and Compositions

Antibodies may be produced using recombinant methods and compositions, e.g., as described in U.S. Pat. No. 4,816,567. In some embodiments, isolated nucleic acid encoding an anti-IL-17 antibody described herein is provided. In some embodiments, isolated nucleic acid encoding an anti-IL-13 antibody described herein is provided. In some embodiments, isolated nucleic acid encoding an anti-IL-13/IL-17 bispecific antibody described herein is provided. Such nucleic acids may encode an amino acid sequence comprising the VL and/or an amino acid sequence comprising the VH of the antibody (e.g., the light and/or heavy chains of the antibody). In some embodiments, one or more vectors (e.g., expression vectors) comprising such nucleic acid are provided. In some embodiments, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody.

In some embodiments, the host cell is eukaryotic, e.g. a Chinese Hamster Ovary (CHO) cell or lymphoid cell (e.g., Y0, NS0, Sp20 cell). In some embodiments, a method of making an antibody is provided, wherein the method comprises culturing a host cell comprising nucleic acid encoding the antibody, as provided above, under conditions suitable for expression of the antibody, and optionally recovering the antibody from the host cell (or host cell culture medium).

In some embodiments, a method of making a multispecific antibody is provided, wherein the method comprises culturing in a host cell comprising nucleic acid encoding the multispecific antibody under conditions suitable for expression of the antibody, and optionaly recovering the multispecific antibody from the host cell (or host cell culture medium). In some embodiments, a method of making a multispecific antibody is provided, wherein the method comprises culturing a first host cell comprising nucleic acid encoding a first VH/VL unit of the multispecific antibody (including constant region, if any, sometimes referred to as a “hemimer” or “half-antibody”) under conditions suitable for expression of the first VH/VL unit, and optionaly recovering the first VH/VL unit from the host cell (or host cell culture medium), and culturing a second host cell comprising nucleic acid encoding a second VH/VL unit of the multispecific antibody (including constant region, if any) under conditions suitable for expression of the second VH/VL unit, and optionaly recovering the second VH/VL unit from the host cell (or host cell culture medium). In some embodiments, the method further comprises assembling the multispecific antibody from an isolated first VH/VL unit and an isolated second VH/VL unit. Such assembly may comprise, in some embodiments, a redox step to form intramolecular disulfides between the two VH/VL units (or hemimers or half antibodies). Nonlimiting exemplary methods of producing multispecific antibodies are described, e.g., in US 2011/0287009, US 2007/0196363, US2007/0178552, U.S. Pat. No. 5,731,168, WO 96/027011, WO 98/050431, WO 2013/055958, WO 2011/133886, and Zhu et al., 1997, Protein Science 6:781-788. A nonlimiting exemplary method is also described in the examples below.

For recombinant production of an anti-IL-17 antibody, anti-IL-13 antibody, or anti-IL-13/IL-17 bispecific antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frupperda cells.

Plant cell cultures can also be utilized as hosts. See, e.g., U.S. Pat. Nos. 5,959,177; 6,040,498; 6,420,548; 7,125,978; and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Exemplary Assays

### Binding Assays and Other Assays

In some embodiments, an antibody provided herein is tested for its antigen binding activity, e.g., by known methods such as ELISA, Western blot, etc.

In some embodiments, competition assays may be used to identify an antibody that competes with an IL-17 antibody described herein for binding to IL-17. In some embodiments, competition assays may be used to identify an antibody that competes with an anti-IL-13/IL-17 bispecific antibody described herein for binding to IL-17 and/or IL-13. In some embodiments, the IL-17 is IL-17A. In some embodiments, the IL-17 is IL-17AF heterodimer. In some embodiments, the IL-17 is IL-17F. In certain embodiments, such a competing antibody binds to the same epitope (e.g., a linear or a conformational epitope) that is bound by an antibody that comprises a VH amino acid sequence comprising SEQ ID NO: 39 and a VL amino acid sequence comprising SEQ ID NO: 38 for binding IL-17. In certain embodiments, such a competing antibody binds to the same epitope (e.g., a linear or a conformational epitope) that is bound by an antibody that comprises a VH amino acid sequence comprising SEQ ID NO: 13 and a VL amino acid sequence comprising SEQ ID NO: 14 for binding IL-13. In certain embodiments, such a competing antibody binds to the same epitope (e.g., a linear or a conformational epitope) that is bound by an antibody that comprises a VH amino acid sequence comprising SEQ ID NO: 30 and a VL amino acid sequence comprising SEQ ID NO: 29 for binding IL-13. In certain embodiments, such a competing antibody is a bispecific antibody that binds to the same epitope (e.g., a linear or a conformational epitope) that is bound by an antibody that comprises a VH amino acid sequence comprising SEQ ID NO: 39 and a VL amino acid sequence comprising SEQ ID NO: 38 for binding IL-17, and binds to the same epitope (e.g., a linear or a conformational epitope) that is bound by an antibody that comprises a VH amino acid sequence comprising SEQ ID NO: 13 and a VL amino acid sequence comprising SEQ ID NO: 14 for binding IL-13. In certain embodiments, such a competing antibody binds to at least 1, 2, 3, 4, 5, 6, 7, 8, 9, or more or all of the amino acid residues of the epitopes. In certain embodiments, such a competing bispecific antibody reduces binding of the bispecific antibody comprising a VH amino acid sequence comprising SEQ ID NO: 39 and a VL amino acid sequence comprising SEQ ID NO: 38 and comprising a VH amino acid sequence comprising SEQ ID NO: 13 and a VL amino acid sequence comprising SEQ ID NO: 14 to IL-13 and/or IL-17 by 10%, 20%, 30%, 40%, 50%, 60%, 70%, 80%, 90% or 100%. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized IL-17 is incubated in a solution comprising a first labeled antibody that binds to IL-17 (e.g., an antibody that comprises a VH amino acid sequence comprising SEQ ID NO: 39 and a VL amino acid sequence comprising SEQ ID NO: 38 (or the corresponding CDRs comprising the amino acid sequences comprising SEQ ID NOS: 40, 43, 44, 45, 46, 47) and a second unlabeled antibody that is being tested for its ability to compete with the first antibody for binding to IL-17. The second antibody may be present in a hybridoma supernatant. As a control, immobilized IL-17 is incubated in a solution comprising the first labeled antibody but not the second unlabeled antibody. After incubation under conditions permissive for binding of the first antibody to IL-17, excess unbound antibody is removed, and the amount of label associated with immobilized IL-17 is measured. If the amount of label associated with immobilized IL-17 is substantially reduced in the test sample relative to the control sample, then that indicates that the second antibody is competing with the first antibody for binding to IL-17. See Harlow and Lane (1988) Antibodies: A Laboratory Manual ch. 14 (Cold Spring Harbor Laboratory, Cold Spring Harbor, N.Y.).

In a further exemplary competition assay, immobilized IL-13 is incubated in a solution comprising a first labeled antibody that binds to IL-13 (e.g., an antibody that comprises a VH amino acid sequence comprising SEQ ID NO: 13 and a VL amino acid sequence comprising SEQ ID NO: 14 (or the corresponding CDRs comprising the amino acid sequences comprising SEQ ID NOs: 15, 16, 17, 18, 19, 20), or an antibody that comprises a VH amino acid sequence comprising SEQ ID NO: 30 and a VL amino acid sequence comprising SEQ ID NO: 29 (or the corresponding CDRs comprising the amino acid sequences comprising SEQ ID NOs: 31, 32, 33, 34, 45, 36) and a second unlabeled antibody that is being tested for its ability to compete with the first antibody for binding to IL-13. The second antibody may be present in a hybridoma supernatant. As a control, immobilized IL-13 is incubated in a solution comprising the first labeled antibody but not the second unlabeled antibody. After incubation under conditions permissive for binding of the first antibody to IL-13, excess unbound antibody is removed, and the amount of label associated with immobilized IL-13 is measured. If the amount of label associated with immobilized IL-13 is substantially reduced in the test sample relative to the control sample, then that indicates that the second antibody is competing with the first antibody for binding to IL-13.

### Activity Assays

In some embodiments, assays are provided for identifying anti-IL-17 antibodies and anti-IL-13/IL-17 bispecific antibodies having biological activity. Biological activity may include, e.g., inhibition of IL-17AA, IL-17AF, and/or IL-17FF binding to IL17Ra and/or Rc; inhibition of IL-17AA-, IL-17AF-, and/or IL-17FF-induced cell proliferation; inhibition of IL-17AA-, IL-17AF-, and/or IL-17FF-induced G-CSF expression; inhibition of IL-17AA-, IL-17AF-, and/or IL-17FF-induced CXCL1, CXCL2, or CXCL3 expression; inhibition of IL-17AA-, IL-17AF-, and/or IL-17FF-induced IL-6 or IL-8 expression; inhibition of IL-17AA-, IL-17AF-, and/or IL-17FF-induced NF-κB expression, activity in inhibiting asthma; and activity in inhibiting idiopathic pulmonary fibrosis (IPF); inhibition of IL-17AA-, IL-17AF-, and/or IL-17FF-induced neutrophil recruitment. In some embodiments, biological activities include, e.g., inhibition of IL-13 binding to an IL-13 receptor (for example, a heterodimeric receptor comprising IL-4Rα and IL-13Rα1), inhibition of IL-13-induced STAT6 phosphorylation, inhibition of IL-13-induced CCL26 expression, inhibition of IL-13-induced cell proliferation, inhibition of IL-13-induced class switching of B cells to IgE, inhibition of IL-13-induced mucus production, activity in inhibiting asthma, activity in inhibiting IPF, and inhibition of IL-13-induced eosinophil recruitment. In some embodiments, biological activities include, e.g., inhibition of IL-13-induced STAT6 phosphorylation, inhibition of IL-13-induced cell proliferation, inhibition of IL-13-induced class switching of B cells to IgE, inhibition of IL-13-induced mucus production, activity in asthma, and activity in IPF, in each case without inhibition of IL-13 binding to an IL-13 receptor (for example, a heterodimeric receptor comprising IL-4Rα and IL-13Rα1). Antibodies having such biological activity in vivo and/or in vitro are also provided. Nonlimiting exemplary assays for testing for such biological activities are described herein and/or are known in the art.

### Immunoconjugates

In some embodiments, immunoconjugates comprising an anti-IL-13/IL-17 bispecific antibody conjugated to one or more cytotoxic agents is provided. Nonlimiting exemplary such cytotoxic agents include chemotherapeutic agents or drugs, growth inhibitory agents, toxins (e.g., protein toxins, enzymatically active toxins of bacterial, fungal, plant, or animal origin, or fragments thereof), and radioactive isotopes.

In some embodiments, an immunoconjugate is an antibody-drug conjugate (ADC) in which an antibody is conjugated to one or more drugs, including but not limited to a maytansinoid (see, e.g., U.S. Pat. Nos. 5,208,020, 5,416,064 and European Patent EP 0 425 235 B1); an auristatin such as monomethylauristatin drug moieties DE and DF (MMAE and MMAF) (see, e.g., U.S. Pat. Nos. 5,635,483 and 5,780,588, and 7,498,298); a dolastatin; a calicheamicin or derivative thereof (see, e.g., U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see, e.g., Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In some embodiments, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In some embodiments, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, R¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See, e.g., WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SLAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### Methods and Compositions for Diagnostics and Detection

In certain embodiments, any of the anti-IL-13/IL-17 bispecific antibodies provided herein is useful for detecting the presence of IL-17 and/or IL-13 in a biological sample. The term “detecting” as used herein encompasses quantitative or qualitative detection. In certain embodiments, a biological sample comprises a cell or tissue, such as serum, plasma, nasal swabs, bronchoalveolar lavage fluid, and sputum.

In some embodiments, an anti-IL-13/IL-17 bispecific antibody for use in a method of diagnosis or detection is provided. In a further aspect, a method of detecting the presence of IL-17 and/or IL-13 in a biological sample is provided. In certain embodiments, the method comprises contacting the biological sample with an anti-IL-13/IL-17 bispecific antibody as described herein under conditions permissive for binding of the anti-IL-13/IL-17 bispecific antibody to IL-17 and/or IL-13, and detecting whether a complex is formed between the anti-IL-13/IL-17 bispecific antibody and IL-17 and/or IL-13. Such method may be an in vitro or in vivo method. In some embodiments, an anti-IL-13/IL-17 bispecific antibody is used to select subjects eligible for therapy with an anti-IL-13/IL-17 bispecific antibody, or any other TH2 pathway inhibitor, e.g. where IL-17 and/or IL-13 is a biomarker for selection of patients.

Exemplary disorders that may be diagnosed using an anti-IL-13/IL-17 bispecific antibody are provided herein.

In certain embodiments, labeled anti-IL-13/IL-17 bispecific antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### Pharmaceutical Formulations

Pharmaceutical formulations of an anti-IL-13/IL-17 bispecific antibody as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In some embodiments, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide a controller and/or TH2 pathway inhibitor with the anti-IL-13/IL-17 bispecific antibody. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### Therapeutic Methods and Compositions

Any of the anti-IL-13/IL-17 bispecific antibodies provided herein may be used in therapeutic methods.

In certain embodiments, the invention provides method of treating eosinophilic and neutrophilic inflammation or disorder in a patient in need thereof. Eosinophilic inflammation is associated with a variety of illnesses, both allergic and non-allergic (Gonlugur (2006) Immunol. Invest. 35(1):29-45). Inflammation is a restorative response of living tissues to injury. A characteristic of inflammatory reactions is the accumulation of leukocytes in injured tissue due to certain chemicals produced in the tissue itself. Eosinophil leukocytes accumulate in a wide variety of conditions such as allergic disorders, helminthic infections, and neoplastic diseases (Kudlacz et al., (2002) Inflammation 26: 111-119). Eosinophil leukocytes, a component of the immune system, are defensive elements of mucosal surfaces. They respond not only to antigens but to parasites, chemicals, and trauma. It has been found that tissue eosinophil and tissue neutrophil counts are positively correlated with serum periostin levels. See, e.g., US 2012/0156194. Patients with eosinophilic inflammation can be identified, in some embodiments, by measuring total serum periostin levels, for example, as described in US 2012/0156194.

As shown herein, IL-17 and neutrophilic inflammation are positively correlated with IL-13 and eosinophilic inflammation in severe uncontrolled asthma. Lung IL-17 is also associated with tissue neutrophil levels in moderate-to-severe asthma in individuals taking inhaled corticosteroids. Further, the IL-17 levels correlate with serum periostin levels.

It was previously reported by Busse et al. that blockade of the IL-17A and IL-17F pathways by an anti-IL-17RA antibody did not produce a treatment effect in subjects with asthma. Busse et al., 2013, Am. J. Respir. Crit. Care Med. 188:1294. The author noted that only in the high-bronchodilator reversibility subgroup was an ACQ change with nominal significance observed, while the significance of the results of the high-reversibility subgroup analysis remains uncertain.

In contrast, the current invention provides methods of treating asthma or a respiratory disorder comprising administering to an individual in need thereof an anti-IL-13/IL-17 bispecific antibody wherein the individual has been determined to have elevated eosinophil count or elevated levels of serum periostin as compared to a reference or control level. Periostin is a Th2 biomarker and patients with elevated levels of periostin are likely to have IL-13-mediated diseases and are likely IL-13-high patient population. As described herein, the therapeutic effect of lebrikizumab, an anti-IL-13 therapeutic antibody, in the periostin-high patient population is heterogeneous. The eosinophil-high (periostin-high) patient population could be further divided to neutrophil-high and neutrophil-low subgroups and that lebrikizumab was more efficacious for the eosinophil-high and neutrophil-low subgroup and less efficacious for the eosinophil-high and neutrophil-high subgroup.

Accordingly, in some embodiments, the invention provides methods for treating asthma or a respiratory disease in an individual comprising administering to the individual an effective amount of an anti-IL13/IL-17 bispecific antibody described herein. In some embodiments, the asthma is moderate to severe asthma. In some embodiments, the individual has high serum periostin. In some embodiments, the individual has an elevated serum periostin as compared to a control or reference level. In some embodiments, the individual further optionally has elevated levels of at least one of CXCL1, IL8, CXCL2, CXCL3, and CSF3. In some embodiments, the individual has elevated serum periostin and elevated levels of CXCL1 as compared to a control or reference level.

In some embodiments, an anti-IL-13/IL-17 bispecific antibody for use in a method of treating an eosinophilic disorder, a neutrophilic disorder, an IL-13 mediated disorder, an IL-17 mediated disorder, and/or a respiratory disorder in an individual is provided. In some embodiments, the method comprises administering to the individual an effective amount of an antibody described herein.

In some such embodiments, a method further comprises administering to the individual a TH2 pathway inhibitor. In some embodiments, the TH2 pathway inhibitor inhibits at least one target selected from ITK, BTK, IL-9, IL-5, IL-13, IL-4, OX40L, TSLP, IL-25, IL-33, IgE, IL-9 receptor, IL-5 receptor, IL-4 receptor alpha, IL-13receptoralpha1 (IL-13Rα1), IL-13receptoralpha2 (IL-13Rα2), OX40, TSLP-R, IL-7Ralpha, IL-17RB, ST2, CCR3, CCR4, CRTH2, FcepsilonRI, FcepsilonRII/CD23, Flap, Syk kinase; CCR4, TLR9, CCR3, IL5, IL3, and GM-CSF. In some embodiments, methods of treating moderate to severe asthma are provided. In some embodiments, methods of treating idiopathic pulmonary fibrosis are provided. In some embodiments, methods of treating an individual with high or elevated serum periostin as compared to a control or reference level are provided. In some embodiments, methods of treating periostin-high asthma are provided. Methods of determining serum periostin levels are provided, for example, in US2012/0156194.

In some embodiments, methods of treating asthma or a respiratory disorder in an individual are provided, wherein the asthma or respiratory disorder are uncontrolled on a corticosteroid. Nonlimiting exemplary corticosteroids include inhaled corticosteroids, such as beclomethasone dipropionate (e.g., Qvar®), budesonide (e.g., Pulmicort®), budesonide/formoterol fumarate dehydrate (e.g., Symbicort®), flunisolide (e.g., Aerobid®), fluticasone propionate (e.g., Flovent®, Flonase®), fluticasone propionate and salmeterol (e.g., Advair®), and triamcinolone acetonide (e.g., Azmacort®). In some embodiments, the patient is also treated with a second controller. The second controller, in some embodiments, may be a long-acting bronchial dialator (LABD). Nonlimiting exemplary long-acting bronchial dilators include long-acting beta-2 agonist (LABA), leukotriene receptor antagonist (LTRA), long-acting muscarinic antagonist (LAMA), theophylline, and oral corticosteroids (OCS). Nonlimiting exemplary LABDs include budesonide/formoterol fumarate dehydrate (e.g., Symbicort®), fluticasone propionate and salmeterol (e.g., Advair®), arformoterol tartrate (e.g., Brovana®), formoterol fumarate (e.g., Foradil®, Performist®), and salmeterol xinafoate (e.g., Serevent®). In certain embodiments, the method further comprises administering to the patient a corticosteroid.

In certain embodiments, an anti-IL-13/IL-17 bispecific antibody for use as a medicament is provided. In certain embodiments, an anti-IL-13/IL-17 bispecific antibody for use in treating asthma, IPF, a respiratory disorder, an eosinophilic disorder, a neutrophilic disorder, an IL-13 mediated disorder, or an IL-17 mediated disorder is provided. In certain embodiments, an anti-IL-13/IL-17 bispecific antibody for use in a method of treatment is provided. In certain embodiments, an anti-IL-13/IL-17 bispecific antibody is provided for use in a method of treating an individual having asthma, a respiratory disorder, an eosinophilic disorder, a neutrophilic disorder, an IL-13 mediated disorder, or an IL-17 mediated disorder comprising administering to the individual an effective amount of the anti-IL-13/IL-17 bispecific antibody. In one such embodiment, the method further comprises administering to the individual an effective amount of at least one additional therapeutic agent, e.g., as described below.

An “individual” or “patient” according to any of the above embodiments is preferably a human. In certain embodiments, the individual or patient is in need for treatment for asthma or a respiratory disorder or is of high risk of developing asthma or a respiratory disorder. In certain embodiments, the asthma patient shows high levels of periostin expression. In certain embodiments, the asthma patient shows elevated levels of periostin as compared to a control or reference level.

In certain embodiments, the patient suffers from moderate to severe asthma.

In certain embodiments, a patient suffering from an eosinophilic inflammation or disorder may exhibit elevated level of one or more of the eosinophilic signature genes as described in 61/894,831, filed on Oct. 23, 2013, and PCT/US14/61759, filed on Oct. 22, 2014, entitled “Methods of Diagnosing and Treating Eosinophilic Disorders”, incorporated herein by reference in its entirety. In certain embodiments, the patient is identified as an Eosinophilic Inflammation Positive (EIP) patient that shows elevated periostin levels and/or elevated levels of one or more selected from CSF1, MEIS2, LGALS12, IDO1, THBS4, OLIG2, ALOX15, SIGLEC8, CCL23, PYROXD2, HSD3B7, SORD, ASB2, CACNG6, GPR44, MGAT3, SLC47A1, SMPD3, CCR3, CLC, CYP4F12, and ABTB2, as compared to a control patient. See US 2012/0156194, incorporated herein by reference in its entirety. Alternatively or additionally, the patient may exhibit elevated levels of one or more of the neutrophilic signature genes such as CXCR1, CXCR2, neutrophil elastase, or CEACAM6.

Among noninvasive biomarkers of the Th2-driven/eosinophilic asthma subphenotype are serum periostin, fractional exhaled nitric oxide (FeNO), and peripheral blood eosinophil count. See Arron et al. (2013) Adv Pharmacol 66: 1-49. In certain embodiments, patients suffering from eosinophilic asthma show high level or elevated level of total serum or plasma periostin, as compared to a control or reference level. In certain embodiments, an EIP patient refers to a patient who had been tested for serum or plasma periostin level, wherein the serum or plasma periostin level is equal to or more than the medium or mean serum or plasma periostin level of a patient population (may also referred to as high periostin). In certain embodiments, the patient who had been tested for serum or plasma periostin level using for example an ELISA or a sandwitch immunoassay as described herein, would have Total Periostin levels of 20 ng/ml or higher (Eosinophilic Positive). In certain embodiments, the patient would have Total Periostin levels of 50 ng/ml or higher. According to certain embodiments, the Total Periostin levels in a patient who is EIP can be selected from the group consisting of 21 ng/ml or higher, 22 ng/ml or higher, 23 ng/ml or higher, 24 ng/ml or higher, 25 ng/ml or higher, 26 ng/ml or higher, 27 ng/ml or higher, 28 ng/ml or higher, 29 ng/ml or higher, 30 ng/ml or higher, 31 ng/ml or higher, 32 ng/ml or higher, 33 ng/ml or higher, 34 ng/ml or higher, 35 ng/ml or higher, 36 ng/ml or higher, 37 ng/ml or higher, 38 ng/ml or higher, 39 ng/ml or higher, 40 ng/ml or higher, 41 ng/ml or higher, 42 ng/ml or higher, 43 ng/ml or higher, 44 ng/ml or higher, 45 ng/ml or higher, 46 ng/ml or higher, 47 ng/ml or higher, 48 ng/ml or higher, 49 ng/ml or higher, 50 ng/ml or higher, 51 ng/ml or higher, 52 ng/ml or higher, 53 ng/ml or higher, 54 ng/ml or higher, 55 ng/ml or higher, 56 ng/ml or higher, 57 ng/ml or higher, 58 ng/ml or higher, 59 ng/ml or higher, 60 ng/ml or higher, 61 ng/ml or higher, 62 ng/ml or higher, 63 ng/ml or higher, 64 ng/ml or higher, 65 ng/ml or higher, 66 ng/ml or higher, 67 ng/ml or higher, 68 ng/ml or higher, 69 ng/ml or higher and 70 ng/ml or higher in the serum or plasma. It should be understood that the EIP Status represents the state of the patient, and is not dependent on the type of assay used to determine the status. Thus, other Eosinophilic Inflammation Diagnostic Assays, including other periostin assays such as the ELISA assay and the ELECSYS® periostin assay shown in US2012/0156194, can be used or developed to be used to test for Eosinophilic Inflammation Status and measure Total Periostin levels. See also Jia et al., 2012, J. Allergy Clin. Immunol. 130:647-654, and US2012/0156194, which are hereby incorporated by reference in their entireties. Exemplary Total Periostin assay procedures are shown below.

The Example 4 of US2012/0156194 (incorporated herein by reference in its entirety) provides a periostin capture ELISA assay (the E4 assay) that is very sensitive (sensitivity 1.88 ng/ml). The antibodies recognize periostin isoforms 1-4 at nM affinity.

The E4 assay: Dilute 80 uL of purified monoclonal antibody, 25D4 (Coat Antibody, SEQ ID NOs: 121 (VH) and 122 (VL) expressed from a hybridoma or a CHO cell line) with phosphate buffered saline to a final concentration of 2 ug/mL. Coat microtiter plates overnight, covered, at 2-8° C. with Coat Antibody 100 μL per well. Wash plate three times with 400 μL wash buffer (PBS/0.05% Tween (polysorbate 20) per well per cycle of wash buffer at room temperature. Add 200 μL per well of blocking buffer to plate. Incubate covered plate at room temp with shaking for 1.5 hours.

Prepare rhuPeriostin standard curve (Standard Stock of rhuPeriostin=rhuPeriostin isoform 1, R&D systems #3548-F2, 5.25 ng/ml, in Assay Diluent (PBS/0.5% bovine serum albumin (BSA)/0.05% polysorbate 20/0.05% ProClin300, pH7.4). Standard curve diluent=PBS/0.5% BSA/0.05% polysorbate 20, 0.05% ProClin300, pH 7.4. For example:

| Std | |
|---|---|
| conc | |
| (pg/mL) | Procedure |
| 600 | 80 μL rhuPeriostin, 5.25 ng/ml in Assay Diluent + 620 μL |
| | standard curve diluent |
| 300 | 300 μL 600 pg/mL rhuPeriostin + 300 μL standard curve diluent |
| 150 | 300 μL 300 pg/mL rhuPeriostin + 300 μL standard curve diluent |
| 75 | 300 μL 150 pg/mL rhuPeriostin + 300 μL standard curve diluent |
| 37.5 | 300 μL 75 pg/mL rhuPeriostin + 300 μL standard curve diluent |
| 18.75 | 300 μL 37.5 pg/mL rhuPeriostin + 300 μL standard curve diluent |
| 9.38 | 300 μL 18.75 pg/mL rhuPeriostin + 300 μL standard curve |
| | diluent |
| 0 | standard curve diluent |

Prepare Controls and samples. Three controls: Spike Source Control (rhuPeriostin full length, isoform 1, R&D Systems #3548-F2), Normal Matrix Control (normal human serum pool, Bioreclamation, Inc.), High Matrix Control (normal human serum pool, plus 100 ng/ml rhuPeriostin spike). For example,

10 μL Control (or sample) serum+1.99 mL sample/control diluent=1:200
300 μL 1:200 dilution+300 μL sample/control diluent=1:400
300 μL 1:400 dilution+300 μL sample/control diluent=1:800
300 μL 1:800 dilution+300 μL sample/control diluent=1:1600
Each dilution is run in singlicate

Construct Matrix Controls using a normal human serum pool. Use unspiked pooled human serum as the Normal Control. Generate the High Control by spiking 100 ng/mL rhuPOSTN into the pooled serum as described above. Compute mean, standard deviation (SD), and % coefficient of variance (CV, expressed in percent) for the four dilutions for each control on every plate. CV is Quantifies magnitude of variance in replicate measurements with respect to mean of replicates. % CV=100*(SD/mean). Evaluate these mean concentrations across all plates to determine inter-plate precision. This control table is then used to define the Normal and High Control pass/fail criteria, setting allowable variance to ±20% of the mean concentration for each control

Wash plate three times with 400 μL per well per cycle of wash buffer (PBS/0.05% polysorbate 20). Add diluted standards (duplicate wells), controls (all four dilutions), and samples (all four dilutions) to plate, 100 μL per well. Incubate plate covered, at room temperature with shaking for 2 hours at room temp. Dilute 80 uL detection MAb stock I (biotinylated murine anti-human periostin, MAb 23B9 (VH: SEQ ID NO:123, VL: SEQ ID NO:124, 7.5 ug/ml in Assay Diluent) to 12 mL with Assay Diluent=50 ng/mL. Wash plate four times with 400 μL per well per cycle of wash buffer. Add diluted detection MAb to plate, 100 μL per well. Incubate covered plate at room temp for one hour with shaking. Dilute 80 uL streptavidin-HRP stock I (AMDEX streptavidin-HRP, GE Healthcare #RPN4401, approximately 1 mg/ml) diluted 1:80 in Assay Diluent to 12 mL with Assay Diluent=1:12 k. Wash plate four times with 400 μL per well per cycle of wash buffer. Add diluted streptavidin-HRP to plate, 100 μL per well. Incubate covered plate at room temp for 45 min. with shaking. Bring Kirkegaard and Perry (KPL) two-step TMB reagents to room temp; do not combine. Wash plate four times with 400 μL per well per cycle of wash buffer. Mix equal volumes of KPL TMB substrate components and add to plate, 100 μL per well. Incubate plate for 20 minutes at room temperature with shaking. Add 1 M phosphoric acid to plate, 100 μL per well. Read plate using 450 nm read wavelength and 650 nm reference wavelength.

A periostin assay using antibodies against isoform 1 (not Total Periostin) was tested on different asthma patient samples using a similar antibody capture format. Preliminary results indicate that periostin isoform 1 is not as robust as a marker for TH2 inflammation as Total Periostin (data not shown).

Alternatively, the quantitative detection of Total Periostin is assessed in an automated Roche cobas e601 ELECSYS® analyzer (Roche Diagnostics GmbH) (the ELECSYS® periostin assay). See Example 7 of US2012/0156194, incorporated herein by reference in its entirety. The test is carried out in the sandwich format wherein the analyte periostin is sandwiched between two monoclonal antibodies binding to two different epitopes on periostin. One antibody is biotinylated and enables the capture of the immuno complex to streptavidin-coated magnetic beads. The second antibody bears a complexed ruthenium cation as the signaling moiety that allows a voltage dependent electrochemiluminescent detection of the bound immuno complex. Exemplary reagents used are shown as follows:
Beads (M): Streptavidin-coated magnetic microparticles 0.72 mg/mL; preservative.
Reagent 1 (R1): Anti-periostin-antibody˜biotin:
   This purified mouse monoclonal-antibody corresponds to the coating antibody 25D4 according to example 4 of US2012/0156194 and is used in biotinylated form >1.0 mg/L; TRIS buffer>100 mmol/L, pH 7.0; preservative.
Reagent 2 (R2): Anti-periostin-antibody˜Ru(bpy):
   This purified mouse monoclonal anti-periostin antibody corresponds to the detection antibody 23B9 according to example 4 of US2012/0156194 and is used in labeled form (labeled with a (Tris(2,2′-bipyridyl)ruthenium(II)-complex (Ru(bpy)) complex)>1.0 mg/L; TRIS buffer>100 mmol/L, pH 7.0; preservative.

The immunoassay can be carried out using two incubations. In the first incubation of about 9 minutes periostin in 20 μL of sample and the biotinylated monoclonal anti-periostin antibody (R1) form a complex. In the second incubation step for further 9 minutes ruthenylated monoclonal anti-periostin antibody (R2) and streptavidin-coated microparticles (M) are added to the vial of the first incubation so that a 3-membered sandwich complex is formed and becomes bound to the solid phase (microparticles) via the interaction of biotin and streptavidin.

The reaction mixture is aspirated into the measuring cell where the microparticles are magnetically captured onto the surface of a platinum electrode. Unbound substances are washed away and the cell flushed with ProCell, a reagent containing Tripropylamine. Application of a voltage to the electrode then induces a chemiluminescent emission which is measured by a photomultiplier.

Results are determined via an instrument-specific calibration curve which is generated by 2-point calibration and a master curve provided via the reagent barcode. Calibrator 1 is analyte free, whereas calibrator 2 contains 50 ng/mL recombinant human periostin in a buffered matrix. To verify calibration, two controls with approximately 30 and 80 ng/mL periostin are employed.

The term “Total Periostin” as used herein refers to at least isoforms 1, 2, 3 and 4 of periostin. Human periostin isoforms 1, 2, 3 and 4 are known in the art as comprising the following amino acid sequences: NP_006466 (SEQ ID NO:109); NP_001129406 (SEQ ID NO:110), NP_001129407 (SEQ ID NO:111), and NP_001129408 (SEQ ID NO:112), respectively, according to the NCBI database, and isoform 5 and has been partially sequenced. Isoform 5 comprises the amino acid sequence of SEQ ID NO:113. In one embodiment, the isoforms of periostin are human periostins. In a further embodiment, the term Total Periostin includes isoform 5 of human periostin in addition to isoforms 1-4. In another embodiment, Total Periostin is Total Serum Periostin or Total Plasma Periostin (i.e., Total Periostin from a serum sample obtained from whole blood or a plasma sample obtained from whole blood, respectively, the whole blood obtained from a patient). In certain embodiments, Total Periostin is measured by the E4 assay or the ELECSYS® assay.

In some embodiments, use of an anti-IL-13/IL-17 bispecific antibody in the manufacture or preparation of a medicament is provided. In one embodiment, the medicament is for treatment of asthma, a respiratory disorder, an eosinophilic disorder, an IL-13 mediated disorder, or an IL-17 mediated disorder. In a further embodiment, the medicament is for use in a method of treating asthma, IPF, a respiratory disorder, an eosinophilic disorder, a neutrophilic disorder, an IL-13 mediated disorder, or an IL-17 mediated disorder comprising administering to an individual having asthma, a respiratory disorder, an eosinophilic disorder, an IL-13 mediated disorder, or an IL-17 mediated disorder an effective amount of the medicament. In one such embodiment, the method further comprises administering to the individual an effective amount of at least one additional therapeutic agent, e.g., as described below.

In some embodiments, pharmaceutical formulations comprising any of the anti-IL-13/IL-17 bispecific antibodies described herein are provided, e.g., for use in any of the above therapeutic methods. In some embodiments, a pharmaceutical formulation comprises any of the anti-IL-13/IL-17 bispecific antibodies provided herein and a pharmaceutically acceptable carrier. In some embodiments, a pharmaceutical formulation comprises any of the anti-IL-13/IL-17 bispecific antibodies provided herein and at least one additional therapeutic agent, e.g., as described below.

Antibodies provided herein can be used either alone or in combination with other agents in a therapy. For instance, an antibody provided herein may be co-administered with at least one additional therapeutic agent. In certain embodiments, an additional therapeutic agent is a TH2 inhibitor and/or a TH17 inhibitor. In certain embodiments, an additional therapeutic is a controller of asthma inflammation, such as a corticosteroid, leukotriene receptor antagonist, LABA, corticosteroid/LABA combination composition, theophylline, cromolyn sodium, nedocromil sodium, omalizumab, LAMA, MABA (e.g., bifunctional muscarinic antagonist-beta2 Agonist), 5-Lipoxygenase Activating Protein (FLAP) inhibitor, or enzyme PDE-4 inhibitor.

Such combination therapies noted above encompass combined administration (where two or more therapeutic agents are included in the same or separate formulations), and separate administration, in which case, administration of the anti-IL-13/IL-17 bispecific antibody can occur prior to, simultaneously, and/or following, administration of the additional therapeutic agent or agents. In some embodiments, administration of the anti-IL-13/IL-17 bispecific antibody and administration of an additional therapeutic agent occur within about one month, or within about one, two or three weeks, or within about one, two, three, four, five, or six days, of each other.

In some embodiments, an anti-IL-13/IL-17 bispecific antibody is used in treating cancer, such as glioblastoma or non-Hodgkin's lymphoma. In some embodiments, antibodies provided herein can also be used in combination with radiation therapy.

An anti-IL-13/IL-17 bispecific antibody (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

An anti-IL-13/IL-17 bispecific antibody would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an anti-IL-13/IL-17 bispecific antibody (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. One skilled in the art can determine a suitable dose of an antibody depending on the type and severity of the disease. Nonlimiting exemplary dosing for anti-IL-13 antibodies is described, e.g., in PCT Publication No. WO 2012/083132. General guidance for dosing of antibodies can be found, for example, in Bai et al., Clinical Pharmacokinetics, 51: 119-135 (2012) and Deng et al., Expert Opin. Drug Metab. Toxicol. 8(2):141-160 (2012). The progress of the antibody therapy may be monitored by conventional techniques and assays.

It is understood that any of the above formulations or therapeutic methods may be carried out using an immunoconjugate in place of or in addition to an anti-IL-13/IL-17 bispecific antibody.

### Articles of Manufacture

In some embodiments, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an anti-IL-13/IL-17 bispecific antibody. The label or package insert indicates that the composition is used for treating the condition of choice. Moreover, the article of manufacture may comprise (a) a first container with a composition contained therein, wherein the composition comprises an anti-IL-13/IL-17 bispecific antibody; and (b) a second container with a composition contained therein, wherein the composition comprises a further cytotoxic or otherwise therapeutic agent. In some embodiments, the article of manufacture may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

All embodiments disclosed herein can be combined with each other unless the context clearly dictates otherwise.

It is understood that any of the above articles of manufacture may include an immunoconjugate in place of or in addition to an anti-IL-13/IL-17 bispecific antibody.

### EXAMPLES

The following are examples of methods and compositions of the invention. It is understood that various other embodiments may be practiced, given the general description provided above.

### Example 1

### IL-17AF and Neutrophilic Inflammation Co-Exist and are Correlated with IL-13 and Eosinophilic Inflammation in Severe Uncontrolled Asthma

The BOBCAT study is a multicenter observational study designed to characterize the relationships between the indices of airway inflammation and noninvasive biomarkers in a cohort of asthma patients. The BOBCAT cohort has been described previously with respect to blood periostin level as an indicator of airway eosinophilia. See Jia et al., 2012, J. Allergy Clin Immunol, 130: 647-654. Study participants were recruited from 18 centers in Canada, US, and Europe. Institutional review boards at each study site approved the protocol, and all subjects provided written informed consent.

Two endobronchial biopsies per patient were fixed in formalin and embedded in a single block of paraffin for subsequent sectioning and staining as previously described. See, e.g., Hauber et al., 2003, J. Allergy Clin. Immunology 112:58-63; Al-Ramli, 2009, J. Allergy Clin Immunol, 123: 1185-1187; Prefontaine et al., 2009, J. Immunol., 183: 5094-5103; Letuve et al., 2006, J. Allergy Clin Immunol, 117: 590-596; Shikotra et al., 2012, J. Allergy Clin Immunol, 129: 104-111. Cells (eosinophils, neutrophils, or positively-staining cells for cytokines as indicated) were counted and expressed as an absolute number per mm² of biopsy tissue.

For all analyses, the distributions of granulocyte levels and positively-staining cells by immunohistochemistry (IHC) in various compartments of the lung were characterized using descriptive statistics as appropriate. Correlations between positively-staining cells are given as Spearman's rank order correlations.

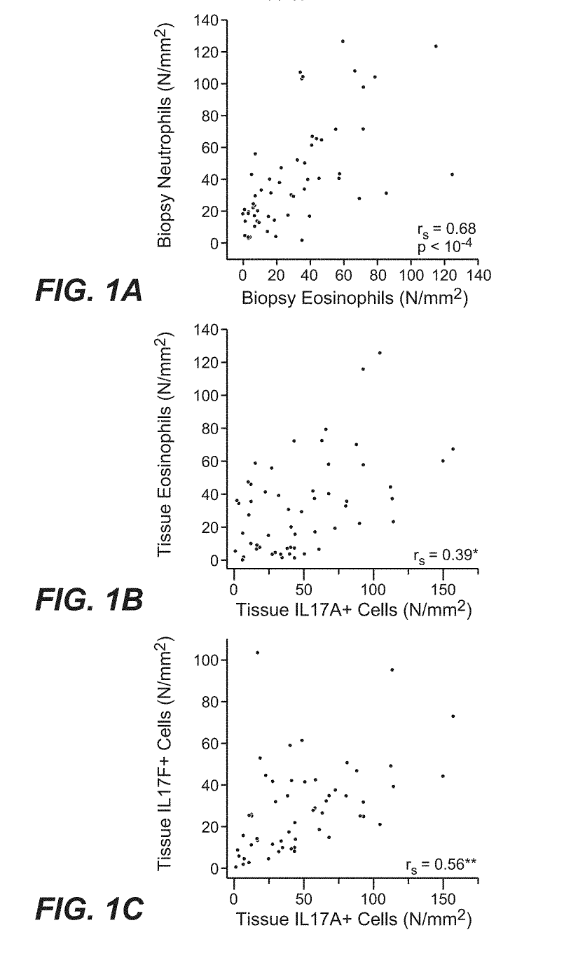

Previous reports have used immunohistochemistry (IHC) to analyze levels of canonical Th17 cytokines IL-17A and IL-17F in bronchial biopsy tissue from severe asthmatics as compared to healthy controls and/or mild asthmatics. See, e.g., Doe et al., 2010, Chest, 138: 1140-1147; Al-Ramli, 2009, J. Allergy Clin Immunol, 123: 1185-1187. IHC for these cytokines was performed in biopsies from the BOBCAT study and examined for the distribution and relationships between the cytokines and granulocytes in matched biopsy tissue. See FIG. 1. Each measurement was detectable across a range of values within the cohort. Significant positive correlations were observed between tissue neutrophil and eosinophil counts (FIG. 1A, rS=0.68, P=4.2×10⁻⁹; see Anon et al, 2014, Eur Respir J, 43:627). In this application, we believe that we show for the first time that in endobronchial biopsies eosinophil counts and the number of cells staining for IL-17A correlate (FIG. 1B, rS=0.39, P=3.2×10⁻³). We also show that in endobronchial biopsies cells staining for IL-17A and IL-17F correlate (FIG. 1C, rS=0.56, P=9.0×10⁻⁶). Previously, airway eosinophils have been shown to positively correlate with serum periostin, blood eosinophils, and exhaled nitric oxide (FeNO). See WO2009/124090 and Jia et al, 2012, J. Allergy Clin Immunol, 130:647-54. In summary, the studies presented here clearly show that in human lung tissue biopsy, tissue eosinophils positively correlated with IL17A⁺ cells, and IL17A⁺ cells positively correlated with IL17F⁺ cells.

### Example 2

### Lung IL-17AF is Associated with Tissue Neutrophils in Moderate-to-Severe Asthma on Inhaled Corticosteroids (ICS)

Asthmatics for the UK Study were recruited and clinical assessments made at the University of Leicester and Queens University of Belfast as described previously. See Shikotra et al., 2012, J. Allergy Clin Immunol, 129: 104-111.

Two endobronchial biopsies per patient were fixed in formalin and embedded in a single block of paraffin for subsequent sectioning and staining as previously described See, e.g., Hauber et al., 2003, J. Allergy Clin. Immunology 112:58-63; Al-Ramli, 2009, J. Allergy Clin Immunol, 123: 1185-1187; Prefontaine et al., 2009, J. Immunol., 183: 5094-5103; Letuve et al., 2006, J. Allergy Clin Immunol, 117: 590-596; Shikotra et al., 2012, J. Allergy Clin Immunol, 129: 104-111. Cells (eosinophils, neutrophils, or positively-staining cells for cytokines as indicated) were counted and expressed as an absolute number per mm² of biopsy tissue.

Primary normal human bronchial epithelial (NHBE) cells were purchased from Lonza (Walkersville, Md.). 6.5 mm diameter 0.4 μM pore density transwell plates from Corning Life Sciences (Corning, N.Y.), were collagen coated using 100 μg/ml PureCol from Advanced BioMatrix (San Diego, Calif.). NHBE were seeded in transwells and maintained in serum-free bronchial epithelial cell growth medium (BEGM, Lonza) for 96 hours or until confluent. The apical media was then removed, and cells were fed basolaterally with Pneumacult complete air liquid interface (ALI) medium (Stem Cell) and differentiated for a period of 21 days.

TaqMan Gene Expression Assays (Applied Biosystems, Foster City, Calif.) were purchased and conducted per the manufacturer's instructions for IL-17A (id: Hs99999082_m1) and IL-17F (id: Hs00369400_m1). Relative expression levels were determined by the 2^{(-ΔΔCT)} method, as described in Applied Biosystems User Bulletin No. 2 (P/N 4303859). Expression levels below the Limit of Quantification (LOQ) were those whose target gene CT (cycle threshold) values were greater than or equal to the lesser value of: 1) 40 or 2) the average CT value of mock RT (reverse transcribed) negative control.

RNA was amplified (MessageAmp II, Ambion, Austin, Tex.) for Agilent (Santa Clara, Calif.) two-color Whole Human Genome 4×44 k gene expression microarrays and performed per manufacturers' instructions, as described in Shikotra et al., 2012, J. Allergy Clin Immunol, 129: 104-111.

For all analyses, the distributions of granulocyte levels and positively staining cells by immunohistochemistry (IHC) in various compartments of the lung were characterized using descriptive statistics as appropriate.

To examine the effects of IL-17A in an in vitro system relevant to the airway, NHBE cells were grown at an air-liquid interface (ALI), which promotes the differentiation of bronchial epithelial cells into a mucociliary pseudostratified epithelium. NHBE cells cultured at ALI were stimulated with IL-17A (10 ng/mL) and TNFα (10 ng/mL) for 24 hours prior to isolation of RNA. Analyses of gene expression microarrays were conducted, and among differentially expressed genes, IL-17A+TNFα stimulation were found to upregulate certain neutrophil-associated genes. See Table 2.

TABLE 2
Neutrophil-associated gene expression in IL-17A + TNFα
stimulated NHBE cells

| | GENE | log₂ | | adjusted |
|---|---|---|---|---|
| GENE NAME | SYMBOL | FC | P-value | P-value |
| chemokine (C—X—C motif) | CXCL1 | 1.54 | 9.29E−05 | 4.12E−03 |
| ligand 1 (melanoma growth | | | | |
| stimulating activity, alpha) | | | | |
| interleukin 8 | IL8 | 1.14 | 4.26E−04 | 0.01 |
| chemokine (C—X—C motif) | CXCL2 | 1.08 | 6.14E−03 | 0.07 |
| ligand 2 | | | | |
| chemokine (C—X—C motif) | CXCL3 | 1.11 | 7.94E−03 | 0.08 |
| ligand 3 | | | | |
| colony stimulating factor 3 | CSF3 | 1.07 | 0.02 | 0.16 |
| (granulocyte) | | | | |

CSF3 encodes granulocyte colony-stimulating factor, a cytokine involved in neutrophil differentiation from hematopoietic precursors. CXCL1, 2, 3, and 8 encode chemokines that bind to CXCR1 and CXCR2, which are receptors expressed on differentiated neutrophils, promoting neutrophil migration.

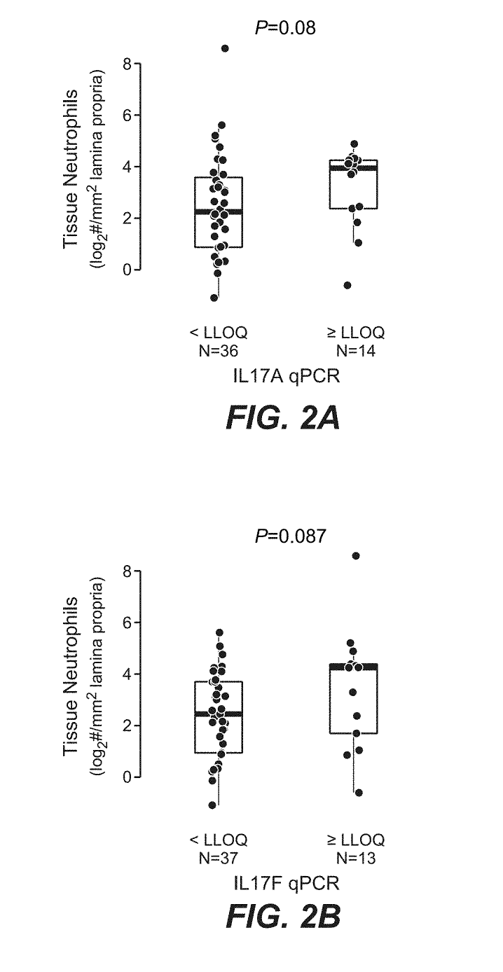

Expression of IL-17A and IL-17F mRNA in bronchial biopsy tissue from the UK cohort was evaluated by qPCR. The expression levels for a majority of samples tested were not detectable: 72% (44 of 61) and 75% (46 of 61) were <Lower Limit of Quantification (LLOQ). We compared available matching tissue neutrophil counts obtained by IHC (50 out of 61 cases) in samples with undetectable (<LLOQ) versus detectable (>LLOQ) IL-17A and IL-17F expression and observed marginally significant elevations. See FIG. 2A (IL-17A; P=0.08) and FIG. 2B (IL-17F; P=0.087). In the UK Cohort, correlation exist between tissue neutrophil counts by IHC and IL-17A and IL-17F expression by qPCR, i.e., IHC samples showing detectable neutrophil counts also show high IL-17A or IL-17F expression by qPCR.

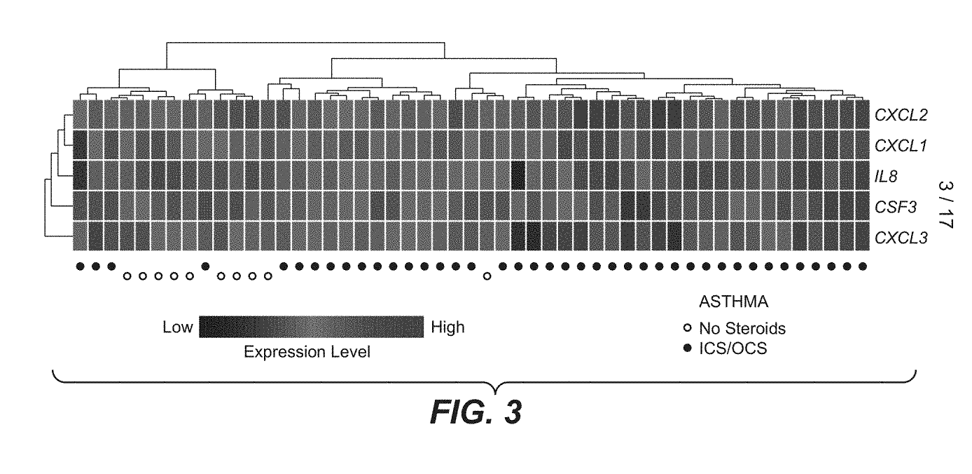

The gene expression of certain IL-17-inducible, neutrophil-associated genes (CXCL1/2/3, IL8, CSF3) were examined by 2-way hierarchical clustering of bronchial biopsy microarray data. See FIG. 3. It was found that the genes were intercorrelated in individual patient samples; that is, subjects could be identified who expressed relatively elevated levels of all of the genes. The subjects with the highest coordinate expression of the genes all had moderate to severe asthma and were taking inhaled corticosteroids (ICS). Further, the set of genes correlated with tissue neutrophils in the UK Cohort (Spearman ρ=0.3902; p=0.0117). The results indicate that elevated neutrophil counts, elevated IL-17A and IL-17F expression, and elevated neutrophil-associated genes expression were observed in a subset of patients taking ICS.

### Example 3

### IL-17AF Biomarkers are Associated with High Serum Periostin

The BOBCAT study and UK Study are described in Examples 1 and 2, respectively.

TaqMan Gene Expression Assays (Applied Biosystems, Foster City, Calif.) were purchased and conducted per the manufacturer's instructions for IL-17A (id: Hs99999082_m1) and IL-17F (id: Hs00369400_m1). Relative expression levels were determined by the 2^{(-ΔΔCT)} method, as described in Applied Biosystems User Bulletin No. 2 (P/N 4303859). Expression levels below the Limit of Quantification (LOQ) were those whose target gene CT values were greater than or equal to the lesser value of: 1) 40 or 2) the average CT value of mock RT negative control.

RNA was amplified (MessageAmp II, Ambion, Austin, Tex.) for Agilent (Santa Clara, Calif.) two-color Whole Human Genome 4×44 k gene expression microarrays and performed per manufacturers' instructions, as described in Shikotra et al., 2012, J. Allergy Clin Immunol, 129: 104-111.

Serum periostin measurements were performed as described previously. See, e.g., Jia et al., 2012, J. Allergy Clin Immunol, 130: 647-654.

A quantitative sandwich enzyme-linked immunosorbent assay (ELISA) kit was characterized to measure CXCL1/GROa concentrations in human plasma samples (R&D Systems Quantikine ELISA kit for the Human CXCL1/GRO alpha Immunoassay, Catalog #DGR00). Briefly, a monoclonal antibody specific to human CXCL1 was coated onto a 96-well microplate. Standards, quality controls, and samples were prepared and added into the wells containing assay diluent. Following incubation at room temperature, the plates were washed to remove any excess unbound reagents, and an appropriate dilution of detection reagent, an anti-human CXCL1 polyclonal antibody conjugated to horseradish peroxidase (HRP), was added. After incubating at room temperature and washing the plates, substrate solution containing 3,3′,5,5′tetramethylbenzidine (TMB) was added for color development. The enzyme reaction was stopped with the addition of sulfuric acid, and the intensity of the color developed was measured at 450 nm, with a reference wavelength at 650 nm. The sensitivity of this assay in human plasma was determined to be 30 pg/mL.

For all analyses, the distributions of granulocyte levels and positively staining cells by immunohistochemistry (IHC) in various compartments of the lung were characterized using descriptive statistics as appropriate.

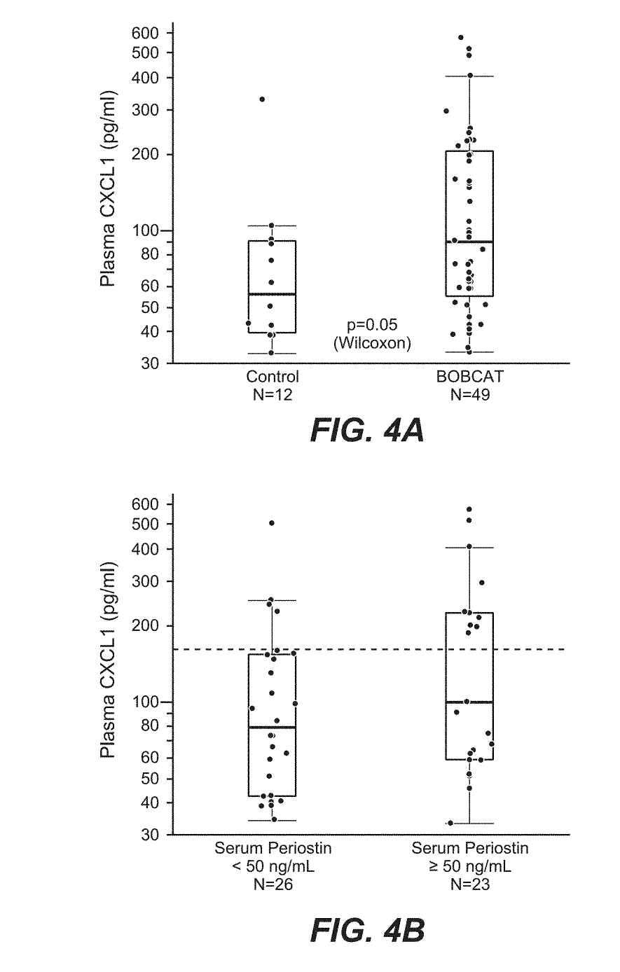

To determine whether protein levels encoded by IL-17-inducible transcripts were detectable at elevated levels in peripheral blood, we assessed plasma CXCL1 levels in the BOBCAT cohort of moderate-severe asthmatics (N=65) and healthy control subjects (N=22). In healthy controls, plasma CXCL1 was below the lower limit of quantitation (LLOQ) in 10/22 subjects (45%) while in asthmatics, plasma CXCL1 was below LLOQ in 16/65 subjects (25%). Among subjects with detectable plasma CXCL1 levels, the levels in asthmatics were significantly higher than healthy controls. See FIG. 4A. Among asthmatic subjects with detectable plasma CXCL1 levels, there was a tendency for those with highly elevated CXCL1 levels (>160 pg/ml) to also have elevated serum periostin levels (>50 ng/ml). See FIG. 4B. The results show that, taken categorically, severe asthmatics with elevated CXCL1 levels in plasma tended to have elevated serum periostin levels (p=0.02 by Fisher's exact test).

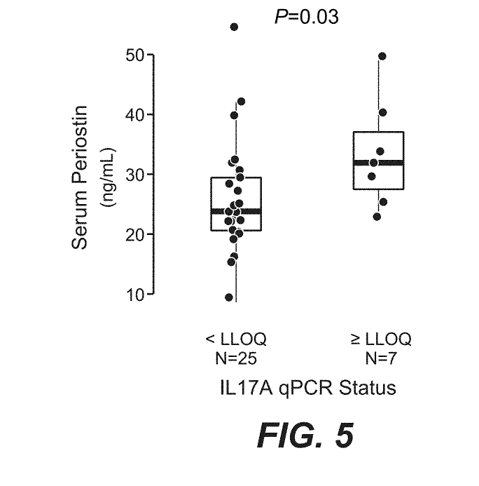

Serum periostin measurements in UK cohort asthmatics were compared to the expression level of IL-17A in matched bronchial biopsies. Subjects with detectable levels of IL-17A mRNA had elevated levels of serum periostin as compared to those whose IL-17A mRNA levels were below the LLOQ (P=0.03). See FIG. 5.

### Example 4

### Independent and Additive Activity of Anti-IL-13 and Anti-IL-17 Antibodies in a Mouse House Dust Mite Asthma Model

The mouse house dust mite asthma model was induced in 8-9 week old C57BL6 mice (Jackson Laboratory, Sacramento, Calif.) by three weekly intranasal challenges with house dust mite extract (Df extract, lot #114218, Greer Labs Inc.) at 100 μg/50 μL PBS. For antibody treatment, an IgG1 control antibody (600 μg control antibody/mouse/injection), an anti-IL-13 IgG1 antibody (Genentech antibody 262A5-1, 200 μg antibody/mouse/injection), and/or an anti-IL-17AA/AF IgG1 antibody (Genentech antibody 16H4.4F3) in combination with an anti-IL-17FF IgG1 antibody (Genentech antibody 28E12) were administered at 200 μg/antibody/mouse (total of 600 μg antibodies/mouse/injection) via intraperitoneal injections 3 times a week during the 2-week sensitization period. 24 hours after the last challenge, blood and bronchoalveolar lavage fluid (BALF) were harvested for total and differential leukocyte counts. Complete blood counts were performed using the Sysmex XT-2000iV automated hematology analyzer. BALF total and differential cell counts were determined by flow cytometry (FacsCaliber3) and manual counting of 200 Wright-Giemsa stained cells under a microscope respectively. The calculated cell numbers were graphed and statistical analyses were performed using Prism software (Graphpad Software, San Diego, Calif.).

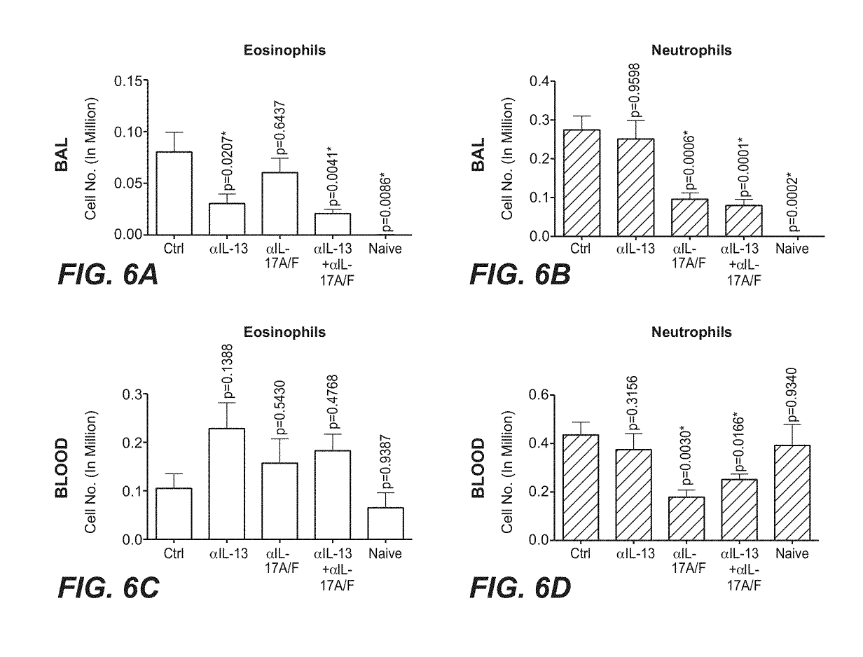

As shown in FIG. 6, calculated eosinophil (A) and neutrophil (B) numbers in BAL showed that anti-IL-13 antibody treatment resulted in decreased eosinophil but unchanged neutrophil numbers while anti-IL-17 antibody treatment (combination treatment with anti-IL-17AA/AF and IL-17FF antibodies) resulted in decreased neutrophil but unchanged eosinophil numbers. The combination treatment with anti-IL-13 and anti-IL-17 antibodies led to reductions in both eosinophil and neutrophil numbers that were consistent with the individual effects of the IL-13 and IL-17 antibodies. The decreases are statistically significant as compared to control. In peripheral blood, anti-IL-13 antibody treatment led to increased eosinophil (FIG. 6C) number, while anti-IL-17 antibody treatment led to a statistically significant decrease in neutrophil (FIG. 6D) numbers without affecting eosinophil numbers. These results show that IL-13 and IL-17AA/AF/FF have independent activities on eosinophils and neutrophils and that combined neutralization of IL-13 and IL-17AA/AF/FF inhibits a broader range of biology than neutralization of either IL-13 or IL-17AA/AF/FF alone. In FIG. 6, * statistically significant.

### Example 5

### Clinical Trial Data Show Heterogeneity in Eosinophil-High Asthma and Response to Lebrikizumab Treatment

The results are consistent with data analyzed from clinical trial samples. Previously we showed that the humanized anti-IL-13 antibody lebrikizumab effectively improved FEV1 in patients with moderate to severe asthma uncontrolled by inhaled corticosteroid therapy, especially in such patients with elevated levels of serum periostin. See Corren et al., 2011, N. Engl. J. Med. 365:1088-98, where periostin-high refers to the level of serum periostin higher than the medium serum periostin within the patient population. As described herein, it was further discovered that although periostin enriched for treatment responses, the response to lebrikizumab within the periostin-high patient group was not homogenous.

We examined Th2 biomarkers including serum periostin, blood eosinophil and FeNO at baseline in combination with base line blood neutrophil level for enrichment of lung function improvement (FEV1) upon IL-13 inhibition in moderate to severe asthmatics in a phase 2 study of lebrikizumab, MILLY. MILLY was a randomized, double-blind, placebo controlled study of lebrikizumab (anti-IL-13) in adults who had asthma that was inadequately controlled despite inhaled glucocorticoid therapy (Corren et al., 2011, N. Engl. J. Med. 365:1088-98). Subjects under consideration were those among the Intent To Treat (ITT) population.

FIG. 15 shows results of percent change in FEV1 in lebrikizumab treated patients that are divided by eosinophil counts and neutrophil counts. Blood eosinophil count was assessed as part of a Complete Blood Cell Count (CBC) on automated hematology analyzers at central laboratories. Blood neutrophil count was assessed as part of a Complete Blood Cell Count (CBC) on automated hematology analyzers. The eosinophil-high group are patients with base line eosinophil count at or above the medium eosinophil count within the same patient population (in this case 210/μl) and the eosinophil-low group are patients with base line eosinophil count below the medium eosinophil count. The neutrophil-high group are patients with base line neutrophil count at or above the medium neutrophil count within the same patient population (in this case 3890/μl) and the neutrophil-low group are patients with base line neutrophil count below the medium neutrophil count.

As shown in FIG. 15, lebrikizumab was more efficacious in patients with a high base line eosinophil count than patients with a low eosinophil count. Compare FIG. 15D with A, B. Within the eosinophil-high group, patients with a low neutrophil count showed marked improvement in percent change in FEV1 by lebrikizumab (FIG. 15D); eosinophil-high patients with a high base line neutrophil count, however, showed reduced benefit by lebrikizumab as compared with patients within the eosinophil-high group that had a low base line neutrophil count. Compare FIG. 15C with D. Similar trends were observed in patients divided by base line periostin levels and FeNO (data not shown).

The results in FIG. 15, together with FIGS. 1B and C, suggest that an anti-IL-13 antibody, for example lebrikizumab, in combination with a neutrophilic antagonist, for example an anti-IL17 antibody, can further benefit patients with moderate to severe asthma and/or further improve efficacy of lebrikizumab. The following describes the generation of a bispecific anti-IL-13/anti-IL-17 antibody.

### Example 6

### Generation of Anti-IL-13/IL-17 IgG4 Bispecific Antibody

We previously established a technology to generate human IgG1 bispecific antibodies with two different light chains in E. coli (Yu et al., 2011, Sci Transl Med 3, 84ra44). The method utilizes knobs-into-holes technology (Ridgway et al., 1996, Protein Eng. 9, 617-621; Atwell et al., 1997, J Mol Biol 270, 26-35) to promote hetero-dimerization of immunoglobulin heavy chains. To enable the use of two different light chains without light chain mispairing, we cultured each arm as a hemimer in separate E. coli cells. We applied this approach to generate the anti-IL-13/IL-17 bispecific antibody by subcloning the anti-IL-17 and anti-IL-13 parental antibodies into vectors allowing the expression of the anti-IL-17 arm as a human IgG4 hole and of the anti-IL-13 arm as a human IgG4 knob. The sequence of the IgG4 knob heavy chain constant region is shown in SEQ ID NO: 69 and the sequence of the IgG4 hole heavy chain constant region is shown in SEQ ID NO: 70.

We based the anti-IL-13 CDRs of the bispecific antibody on lebrikizumab, which has been previously generated and characterized. See, e.g., PCT Publication No. WO 2005/062967 A2. Lebrikizumab binds soluble human IL-13 with a Biacore-derived Kd that is lower than the detection limit of 10 pM. Binding of lebrikizumab to IL-13 does not inhibit binding of the cytokine to IL-13Rα1, but does block the subsequent formation of the heterodimeric signaling competent IL-4Rα/IL-13Rα1 complex (Ultsch, M. et al., 2013, J. Mol. Biol., dx.doi.org/10.1016/j.jmb.2013.01.024; Corren et al., 2011, N. Engl. J. Med. 365, 1088-1098). For the bispecific antibody, the anti-IL-13 antibody had two deviations in the FR region as compared to lebrikizumab: Q1E on heavy chain and M4L on the light chain. See SEQ ID NOs: 13 and 14, respectively. The two changes were combined in a single anti-IL-13 half antibody, and the resulting half antibody was found to have improved yield and folding over the wild-type anti-IL-13 half-antibody.

For antibody expression, E. coli strain 64B4 was used. An overnight culture was grown at 30° C. in LB (100 μg/ml carbenicillin), diluted 1:100 into 5 ml CRAP media (100 μg/ml carbenicillin) (Simmons et al., 2002, J. Immunol. Methods, 263: 133-147) and grown for 24 hours at 30° C.

For non-reduced analysis by SDS-PAGE, 200 μl of CRAP expression culture was pelleted and resuspended in 100 μl NR-lysis buffer (88 μl PopCulture Reagent (Novagen), 10 μl 100 mM iodoacetamide, 2 μl lysonase reagent (EMD Biosciences)). Samples were incubated 15 minutes at room temperature, then spun at 9300 rcf for 5 minutes to pellet insoluble components. 50 μl supernatant was transferred to a new tube and mixed with 50 μl 2× LDS sample buffer (Invitrogen). Samples were then heated for 5 minutes at 95° C. and 5 μl was loaded on NuPAGE 4-12% Bis-Tris/MES gels (Invitrogen). Gels were transferred by iBlot (Invitrogen) onto nitrocellulose membrane, immunoblotted with IRDye800CW conjugated anti-human (H&L) antibody (Rockland) and imaged with a LiCOR Odyssey Imager.

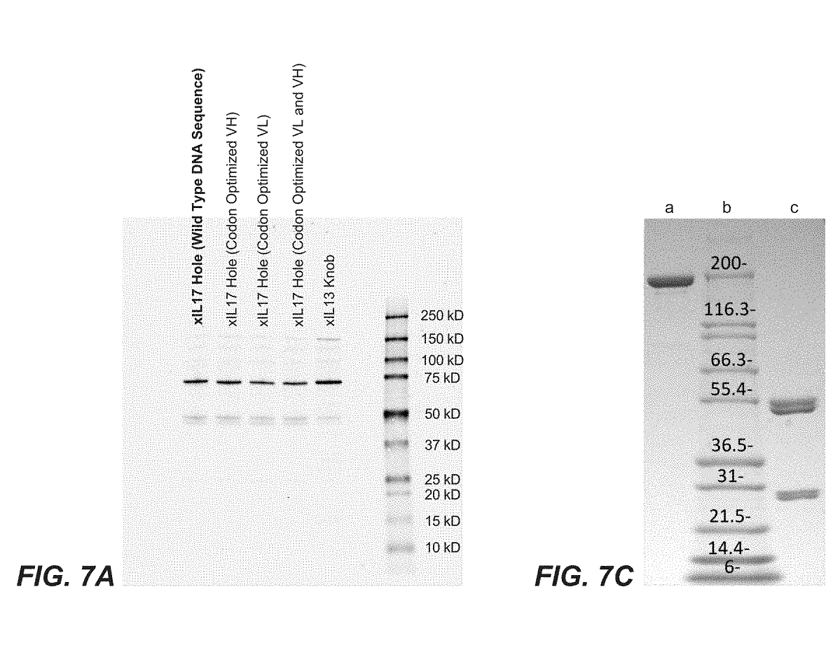

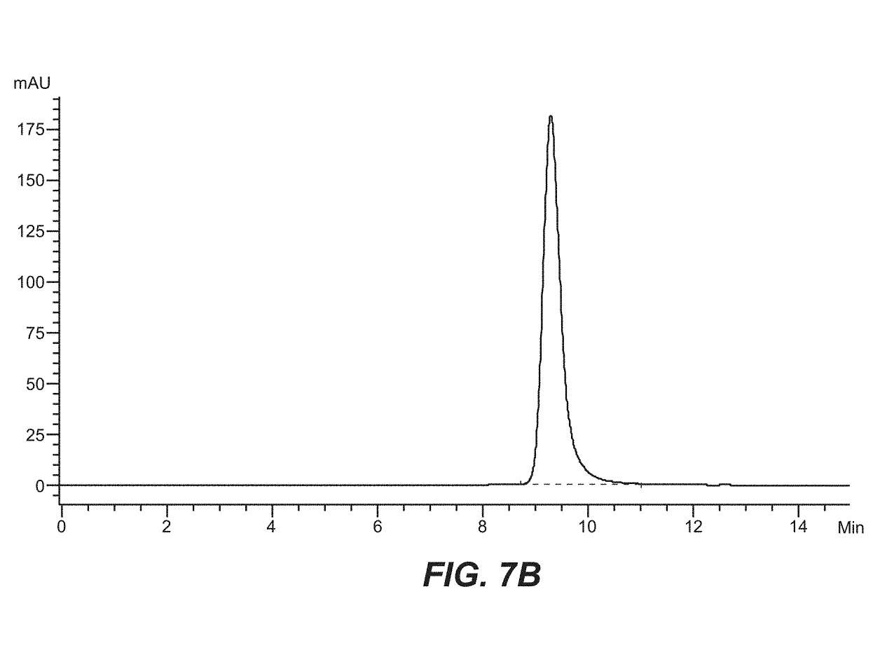

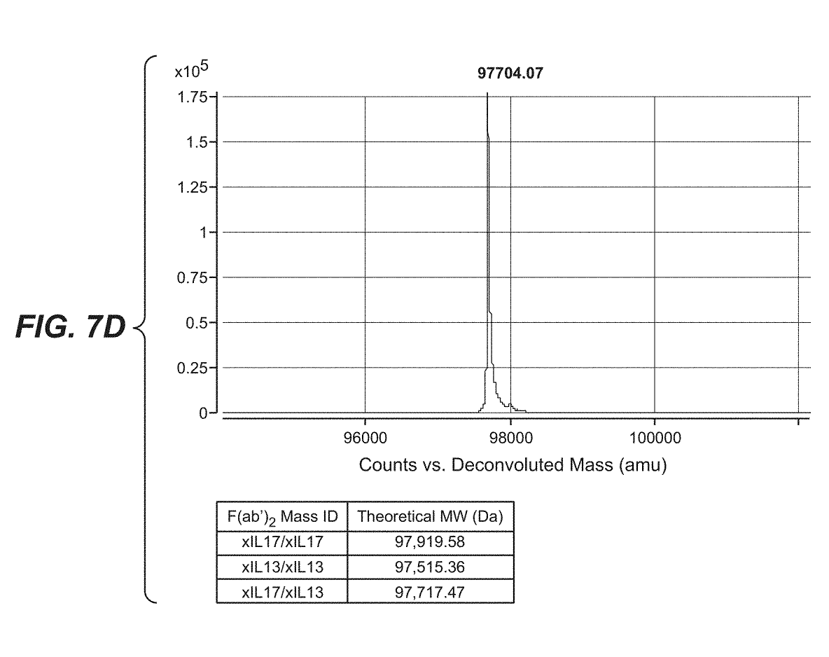

FIG. 7A shows SDS-PAGE analysis of the knob and hole half antibodies. Codon optimized versions of the heavy and light chains for the IL-17 half antibody were also made and tested for expression. The sequences of the original coding sequences and the codon optimized coding sequences are shown in SEQ ID NOs: 99 to 102. The CDRs of the anti-IL17 half antibody are shown in SEQ ID NOs:40, 43, 44, 45, 46 and 47. See US 2012/0141492 or U.S. Pat. No. 8,715,669. In each culture, the half-antibody species was the predominant band. The original coding sequences were selected for scaling up the antibody production.

For scale-up to 10 L fermenters, initial starter cultures (500 ml) were grown into stationary phase and used to inoculate 10 L fermentations (Simmons et al., 2002, J. Immunol. Methods, 263: 133-147). 10 L fed-batch cultures were grown and whole broths were harvested via microfluidics. The lysed cells were then treated overnight at 4° C. with a final concentration of 0.8% PEI (v/v). Each mixture was subsequently centrifuged at 15,000×g for 20 minutes followed by filtration through a 0.22 μm filter. Each half antibody was then captured on a 250 mL MabSURE SELECT column (GE Healthcare Life Sciences). The column was equilibrated with 10 column volumes (CV) of an equilibration buffer consisting of 50 mM TRIS pH 8.0, 150 mM NaCl, followed by washes with two different wash buffer, the first consisting of 50 mM TRIS pH 8.0, 150 mM NaCl, 0.05% Triton X-100, 0.05% Triton X-114, and the second consisting of 25 mM Sodium Citrate pH 6.0. Each arm was eluted into 0.15 M Sodium Acetate pH 2.7, then titrated to pH 5.0 using 1:10 1M Arginine/Succinate pH 8.7.

The identity of each half antibody was confirmed by liquid chromatography electrospray ionization with time-of-flight (LC-ESI/TOF) analysis. Purity was analyzed by 4-20% Tris-Glycine SDS PAGE gel. Aggregate levels were determined by SEC.

During the initial assessment, we found that the anti-IL-17 half-antibody formed precipitates at pH higher than 7. The half-antibody was captured and then eluted from a protein A column at low pH and the eluate was adjusted to pH 8.5 to carry out the assembly of bispecific antibody in a redox reaction. About 20% of the anti-IL-17 half antibody eluate was lost to precipitation after pH adjustment. The decrease in available anti-IL-17 half antibody to pair with anti-IL-13 half antibody led to an imbalance between the ratio of the two half antibodies and reduced the yield of anti-IL-13/IL-17 bispecific. Following assembly, the bispecific antbody was purified by cation exchanger chromatography. Unlike other bispecific antibodies, however, some of the anti-IL-13/IL-17 bispecific antibody irreversibly bound to the cation ion exchanger resin SPHP. As a result of the above observations, the percentage of bispecific formed was unusually low at about 10%.

Several conditions were tested to improve the solubility and stability of the anti-IL-17 half-antibody before and during assembly, for example, adding to the eluted half antibody different concentrations of arginine at 100 mM, 250 mM, 350 mM and 500 mM, adding 4% polyvinylpyrrolidone (PVP), and/or increasing pH from pH 8.5 to 10 in 0.5 pH unit increments. High concentration of arginine (0.5 M) at about pH 9 was found to greatly increase the solubility of the IL-17 half-antibody and to reduce pH-induced precipitation to less than 1%. 500 mM arginine at pH 8.5 also led to good results. We also found that the percentage of bispecific formed can be increased by combining the anti-IL-13 half-antibody with the anti-IL-17 half antibody before pH adjustment for disulfide oxidation by the addition of 0.5 M arginine at pH 8.5. In addition, we replaced the cation exchanger column with a hydrophobic interaction column because the anti-IL-13/IL-17 bispecific did not irreversibly bind to a hydrophobic interaction column. When following the improved processes, the percentage of bispecific antibody formed increased from 10% to 65%.

In one exemplary experiment, anti-IL-17 half antibody was combined with anti-IL-13 at a 1:1 ratio then titrated to 0.5M Arginine/Succinate pH 8.7, after which freshly prepared reducing agent, reduced L-glutathione (GSH), was added to achieve a molar ratio of 1:200. The mixture was left at room temperature for three days. Following redox, the assembled bispecific was purified on a 45 mL HIC ProPac 10 column (Thermo Scientific) using a 30 CV gradient. The running buffer was 25 mM potassium phosphate, 1 M ammonium sulfate pH 6.5 and the elution buffer was 25 mM potassium phosphate pH 6.5, 25% isopropanol. Thirty mL fractions were collected and peak fractions were separated by 4-20% Tris-Glycine SDS PAGE to analyze purity and pooled accordingly. The pool was then concentrated to 10 mg/mL and dialyzed into PBS. The concentrated bispecific pool was further purified to remove endotoxins by column chromatography. Unexpectedly, aggregates were again observed during the purification process. To reduce aggregates, Triton X-114 was spiked into the protein pool at a final concentration of 0.1% (v/v). The pool was mixed thoroughly and incubated on ice for 5 minutes, followed by heating at 37° C. for 15 minutes. Subsequently, the mixture was centrifuged for 10 minutes at 25° C. at 3,000×g and the aqueous layer was aspirated and passed over gel filtration to remove remaining Triton X-114. The addition of Triton X-114 removed all detectable aggregates.

Purity was analyzed by 4-20% Tris-Glycine SDS PAGE gel and aggregate levels were determined by SEC. The identity of the assembled bispecific was confirmed by LC-ESI/TOF in its intact and reduced form. Since the theoretical homodimer and heterodimer masses are within a few Daltons of each other, the bispecific was also analyzed after Fc removal using Fabricator. The intact antibody was treated with 1 unit of Fabricator per 1 ug of protein at pH 6.5 and incubated at 37° C. for 4 hours. The identity of the F(ab′)₂ was confirmed by LC-ESI/TOF.

FIG. 7B shows the SEC analysis, which reveals a single predominant species. FIG. 7C shows SDS PAGE analysis of the bispecific antibody under (lane a) nonreducing and (lane c) reducing conditions. Lane b shows molecular weight markers. FIG. 7D shows LC-ESI/TOF analysis of the F(ab′)₂ fragments, and the theoretical molecular weights for the IL-17 homodimer, IL-13 homodimer, and anti-IL-13/IL-17 bispecific F(ab′)₂.

### Example 7

### Cytokine Binding Affinity of anti-IL-13/IL-17 Bispecific Antibody

Binding affinities of the anti-IL-17/anti-IL-13 bispecific antibody against human and cynomolgus monkey IL-13 cytokines were measured with a BIAcore™-T200 instrument. Anti-IL-13 antibody was captured by mouse anti-human Fc antibody (GE Healthcare, cat #BR-1008-39) coated on CM5 biosensor chips to achieve approximately 500 response units (RU). Four-fold serial dilutions (50 nM to 49 pM) of human IL-13, human IL-13R130Q (a common IL-13 variant associated with allergy and asthma, see Vladich et al., 2005, J. Clin Invest., 115:747-754), and cyno IL-13 were injected in FIBS-P buffer (GE Healthcare) at 25° C. with a flow rate of 30 μl/min. Association rates (kₒₙ) and dissociation rates (k_{off}) were calculated using a simple one-to-one Langmuir binding model (BIAcore T200 Evaluation Software version 2.0). The equilibrium dissociation constant (K_{D}) was calculated as the ratio k_{off}/kₒₙ. The results are shown in Table 3.

TABLE 3
Biacore association and dissociation rates of the anti-IL-13/IL-17
bispecific antibody

| Ligand | kₒₙ (M⁻¹s⁻¹) | k_{off} (s⁻¹) | K_{d} (M) |
|---|---|---|---|
| human IL-13 | 5.84 × 10⁵ | 2.07 × 10⁻⁵ | 3.55 × 10⁻¹¹ |
| human IL-13 | 8.64 × 10⁵ | <1 × 10⁻⁶ | <1 × 10⁻¹² |
| R130Q | | | |
| cyno IL-13 | >1 × 10⁶ | <1 × 10⁻⁶ | <1 × 10⁻¹² |

Binding affinities of anti-IL-17/anti-IL-13 bispecific antibody against various IL-17 cytokines were measured by Surface Plasmon Resonance (SRP) using a BIAcore™-T200 instrument. Anti-IL-17/anti-IL-13 bispecific antibody was captured by mouse anti-human Fc antibody (GE Healthcare, cat #BR-1008-39) coated on CM5 biosensor chips to achieve approximately 500 response units (RU). For kinetics measurements, five-fold serial dilutions (20 nM to 32 pM) of human IL-17AA (R&D Systems, cat #317-ILB-050), human IL-17AF heterodimer, human IL-17FF (R&D Systems, cat #1335-IL-025/CF), cyno IL-17AA, cyno IL-17AF, and cyno IL-17FF were injected in HBS-P buffer (GE Healthcare) at 25° C. with a flow rate of 30 μl/min. Association rates (kₒₙ) and dissociation rates (k_{off}) were calculated using a simple one-to-one Langmuir binding model (BlAcore T200 Evaluation Software version 2.0). The equilibrium dissociation constant (K_{D}) was calculated as the ratio k_{off}/kₒₙ. The results are shown in Table 4.

TABLE 4
Biacore association and dissociation rates of the anti-IL-13/IL-17
bispecific antibody

| Ligand | kₒₙ (M⁻¹s⁻¹) | k_{off} (s⁻¹) | K_{d} (pM) |
|---|---|---|---|
| human IL-17AA | 5.55 × 10⁶ | 1.18 × 10⁻⁴ | 21 |
| human IL-17AF | 4.78 × 10⁶ | 5.97 × 10⁻⁶ | 1.3 |
| human IL-17FF | >1 × 10⁷ | 2.59 × 10⁻⁴ | <26 |
| cyno IL-17AA | 8.89 × 10⁶ | 2.91 × 10⁻⁴ | 33 |
| cyno IL-17AF | >1 × 10⁷ | 6.81 × 10⁻⁵ | <7 |
| cyno IL-17FF | >1 × 10⁷ | 9.33 × 10⁻⁴ | <93 |

### Example 8

### Inhibition of Cytokine-Induced Proliferation by Anti-IL-13/IL-17 Bispecific Antibody

The effect of the anti-IL-13/IL-17 bispecific antibody on IL-13-induced activity in TF-1 cells was studied as follows. Human TF-1 cell (erythroleukemic cells, R&D Systems, Minneapolis, Minn.) were cultured in a humidified incubator at 37° C. with 5% CO₂ in RPMI 1640 growth media containing 10% heat inactivated fetal bovine serum (FBS) (Catalog No. SH30071.03, HyClone Laboratories, Inc., Logan, Utah); and 1× Penicillin: Streptomycin:Glutamine (Catalog No. 10378-016, Gibco Invitrogen Corp., Carlsbad, Calif.) and 2 ng/mL rhGM-CSF (Catalog No. 215-GM, R&D Systems, Minneapolis, Minn.). Assay media is growth media without 2 ng/mL rhGM-CSF. Cytokines were added to the assay media as specified at the following final concentrations, 10 ng/ml human IL-13 or 10 ng/ml human IL-13 R130Q.

Antibodies were serially diluted 3.3 fold in assay media containing human cytokines in a 96 well tissue culture plate (Catalog No. 353072, Falcon BD, Franklin Lakes, N.J.). Plates were incubated for 20 minutes at 37° C. TF-1 cells were washed twice in assay media and resuspended at a final volume of 2.5×10⁵ cells/ml, 50 μl of the TF-1 cells are added to each well. The total volume per well was 100 μL. Plates were incubated for 4 days in a humidified incubator at 37° C. with 5% CO₂ before the addition of 1 μCi of ³H-thymidine per well. After an additional 4 hours of incubation, proliferation was measured by ³H-thymidine incorporation. Cell-associated radioactivity was quantified by scintillation counting. Results are expressed as the mean of duplicate samples. Graphs were generated and statistical analysis was performed using KaleidaGraph (Synergy Software, Reading, Pa.).

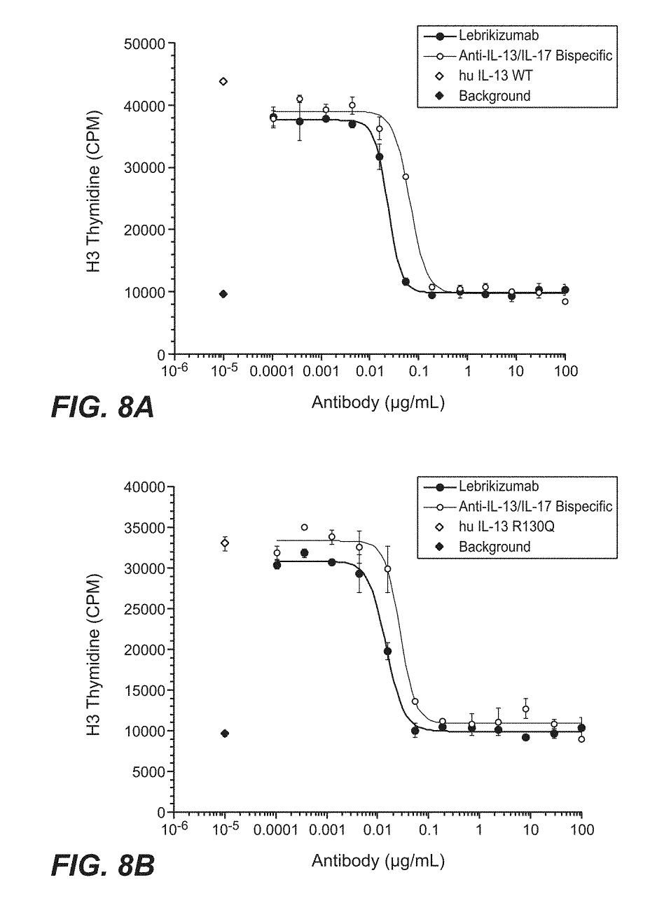

The results of that experiment are shown in FIG. 8. The anti-IL-13/IL-17 bispecific antibody inhibited IL-13-induced (FIG. 8A) and IL-13 R130Q-induced (FIG. 8B) proliferation of TF-1 cells in a dose dependent manner, with comparable potencies to lebrikizumab. Table 5 shows the IC90 results for each antibody and each cytokine.

TABLE 5
IC90 values for lebrikizumab and the anti-IL-13/IL-17 bispecific antibody

| | | Human |
|---|---|---|
| | Human IL-13 | IL-13 R130Q |
| | IC90 (μg/ml) | IC90 (μg/ml) |
| Lebrikizumab | 0.05 | 0.03 |
| Anti-IL-13/IL-17 bispecific antibody | 0.15 | 0.06 |

The effect of the anti-IL-13/IL-17 bispecific antibody on IL-17-induced proliferation of normal human foreskin fibroblast cells was studied as follows. A frozen aliquot of Normal Human Foreskin Fibroblast (NHFF) was obtained from Life Technologies (Catalog No. C-004-5C, Life Technologies, Carlsbad, Calif.) and was expanded in medium 106 (Catalog No. M106500, Life Technologies, Carlsbad, Calif.) supplemented with Low Serum Growth Supplement (LSGS; Catalog No. 500310, Life Technologies, Carlsbad, Calif.). Recombinant human IL-17AA and IL-17FF were obtained from R&D Systems (IL-17AA: 317-ILB-050 and IL-17FF: 1335-IL-025/CF) and reconstituted in 4 mN HCl per manufacturer's instruction. Recombinant human IL-17AF was generated and purified in house.

The day before the assay was performed, aliquots of NHFF were thawed and seeded at 0.125×10⁶ cells/well in 100 μL of LSGS-supplemented medium 106 in 96-well flat bottom tissue culture plates and were incubated overnight at 37° C. for attachment. In the morning of the assay, media was replaced with fresh media at 100 μL/well and the cells were further incubated for 2+ hours for equilibration.

The antibodies were diluted 1:3 serially starting from 50 μg/ml as the highest concentration. A 10× working stock was made at 500 μg/ml and the serial dilution was performed in tissue culture media. The cytokines were also prepared as 10× working stock in tissue culture media. IL-17AA was diluted to 16 ng/ml for the final concentration of 1.6 ng/ml, IL-17AF at 1.25 ug/ml for the final concentration of 125 ng/ml, and IL-17FF at 15 ug/ml for the final concentration of 1.5 ug/ml. The working stocks of antibodies and the cytokines were added, each at 10 μL/100 μL/well and incubated for 24 hours. The supernants were harvested and transferred to fresh 96-well round bottom plates and frozen at −80° C. until analysis.

G-CSF ELISA was performed using a commercial kit purchased from R&D Systems (Minneapolis, Minn. Catalog. No. DY214) per manufacturer's instructions. The data were analyzed using Excel (Microsoft, Redmond, Wash.) and Prism (Graphpad Software, San Diego, Calif.) software to calculate the IC50 and IC90 values.

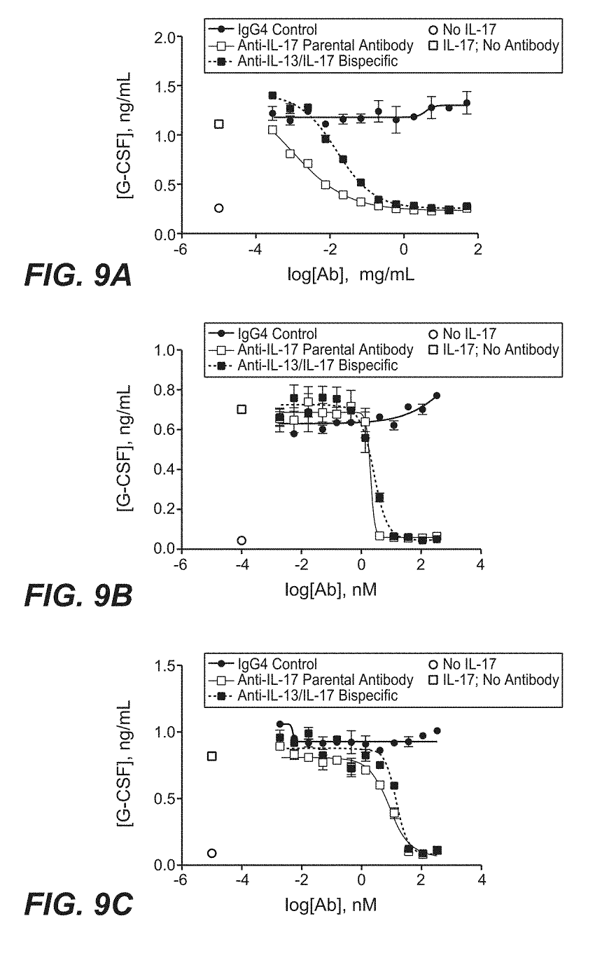

The results of that experiment are shown in FIG. 9. IL-17AA (1.6 ng/ml, equivalent to 0.05 nM), IL-17AF (125 ng/ml, equivalent to 4 nM), and IL-17FF (1.5 μg/ml, equivalent to 50 nM) elicit cytokine-dependent G-CSF expression in NHFF cells (see open square in FIGS. 9A, B, and C, respectively). The IgG4 control antibody had no effect on G-CSF expression, while both the anti-IL-17 parental antibody and anti-IL-13/IL-17 bispecific antibody inhibited G-CSF expression induced by all three isoforms of IL-17 in a dose-dependent manner. Table 6 shows the IC90 for inhibition of IL-17 induced G-CSF expression by the anti-IL-13/IL-17 bispecific antibody.

TABLE 6
IC90 values for the anti-IL-13/IL-17 bispecific antibody

| | IL-17AA | | IL-17FF |
|---|---|---|---|
| | IC90 | IL-17AF | IC90 |
| | (μg/ml) | IC90 (molar ratio) | (molar ratio) |
| anti-IL-13/IL-17 bispecific | 0.19 | ~2 | ~1 |
| antibody | | | |
| Parental anti-IL17 antibody | 0.04 | ~1 | ~1 |

IL-17AF heterodimer and IL-17F homodimer require high cytokine concentrations to elicit a robust response. The IC90 for inhibition of that response occurs at a molar ratio of antibody:cytokine of ˜2 and ˜1 for IL-17AF and IL-17F, respectively.

### Example 9

### Neutralization of Both IL-13 and IL-17 by Anti-IL-13/IL-17 Bispecific Antibody

The activity of the anti-IL-13/IL-17 bispecific antibody to neutralize both IL-13 and IL-17 in the same assay was assessed in a BEAS-2B cell assay. CCL26 mRNA levels were used to assess the IL-13 response, and CXCL1 secretion was measured by ELISA to assess the IL-17 response.

BEAS-2B human bronchial epithelial cells were obtained from ATCC (Catalog No. ATCC CRL-9609, Manassas, Va.) and grown in collagen-treated tissue culture flasks in fully supplemented BEGM media (Catalog No. CC-3170, Lonza, Walkersville, Md.). Recombinant human IL-13 was generated in house, and recombinant human IL-17AA (Catalog No. IL-17AA: 317-ILB-050) and TNFα (Catalog No. 210-TA-005/CF) were obtained from R&D Systems (Minneapolis, Minn.).

Frozen aliquots of BEAS-2B cells were thawed and seeded at 10⁴ cells/well in collagen-treated 96-well flat bottom plates in complete BEGM media and allowed to expand for 2-3 days until confluence was reached. The media was then replaced with fresh BEGM media lacking hydrocortisone and cultured for another 2-3 days for steroid withdrawal. On the day of the assay, media was replaced with 100 μL/well fresh BEGM lacking hydrocortisone and equilibrated for a few hours at 37° C.

Antibody dilutions were performed as described above for the IL-17 fibroblast cell assay. For cytokines, 10× working stocks of IL-13 (100 ng/ml), IL-17AA (16 ng/ml), and TNFα (1 ng/ml) were prepared and added to the wells for the final concentrations of 10 ng/ml, 1.6 ng/ml, and 0.1 ng/ml, respectively, and cultured at 37° C. Twenty-four hours later, the supernatants were harvested and frozen at −80° C., and the rest of the media in the wells were aspirated and the wells containing the attached cells were washed with cold RNase-free PBS once, aspirated, and processed for cDNA synthesis using the Cells-to-cDNA II kit (Catalog No. AM1723, Life Technologies, Carlsbad, Calif.) according to the manufacturer's protocol.

Taqman primers and probe for human CCL26 were purchased from Life Technologies (Catalog No. 4331182; Assay ID: Hs00171146_m1) and those for the internal control RPL19 were designed and generated in house (Forward primer 5′-AGC GGA TTC TCA TGG AAC A-3′ (SEQ ID NO: 96); Reverse primer 5′-CTG GTC AGC CAG GAG CTT-3′ (SEQ ID NO: 97); and Probe 5′-TCC ACA AGC TGA AGG CAG ACA AGG-3′ (SEQ ID NO: 98)). The Taqman qPCR reaction was set up in 20 μL volume/reaction using the TaqMan Universal PCR Master Mix (Catalog No. 4304437, Life Technologies, Carlsbad, Calif.) and the PCR reactions were run using the Applied BioSystems 7500 Real Time PCR System. The relative quantity (RQ) values were calculated as the ratio of normalized delta cycle threshold (dCT) values of the experimental value over the no stimulation condition, which was treated as the baseline response. The RQ values were plotted against log[Ab] in Prism to calculate the IC90 values.

Supernatants from the BEAS-2B culture were analyzed for CXCL1 by ELISA using a kit purchased from R&D Systems (Catalog. No. DY275) per manufacturer's instructions. The data were analyzed using Excel and Prism software to calculate the IC50 and IC90 values. The concentration of CXCL1 was plotted against the log[Ab] in Prism to calculate the IC90 values.

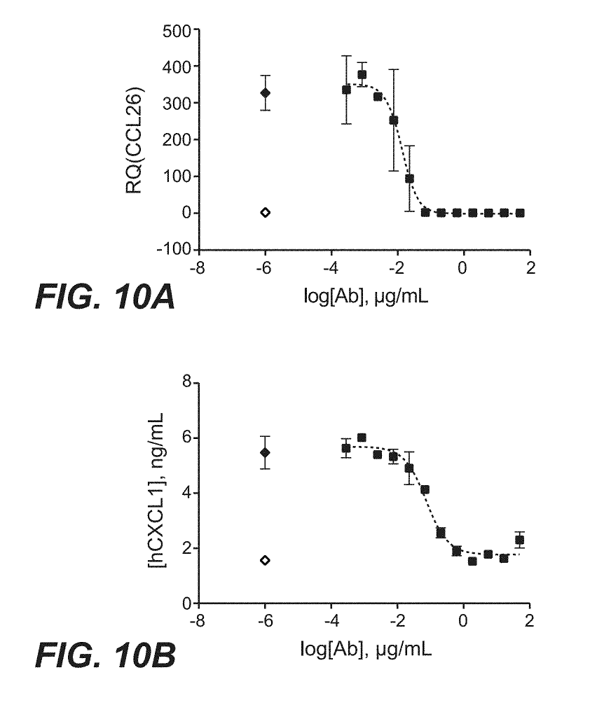

The results of that experiment are shown in FIG. 10. The anti-IL-13/IL-17 bispecific antibody inhibited both CCL26 expression (FIG. 10A) and CXCL1 expression (FIG. 10B) in a dose-dependent manner, suggesting that the bispecific antibody can neutralize both IL-13 and IL-17 in the same assay. IC90 values were calculated by non-linear regression and are shown in Table 7.

TABLE 7
IC90 values for the anti-IL-13/IL-17 bispecific antibody

| | IL-13 | IL-17AA |
|---|---|---|
| | IC90 (μg/ml) | IC90 (μg/ml) |
| anti-IL-13/IL-17 bispecific antibody | 0.04 | 0.19 |

The IC90 value for CCL26 expression (0.04 μg/ml) is similar to the IC90 of the bispecific antibody in the IL-13 assay described above (see Table 5), while the IC90 value for CXCL1 expression (0.19 μg/ml) is similar to the IC90 for the bispecific antibody in the IL-17 cell assay described above (see Table 6).

### Example 10

### Pharmacokinetics of the Anti-IL-13/IL-17 Bispecific Antibody Following Administration to Mice

The pharmacokinetics of anti-IL-13/IL-17 bispecific antibody after a single intravenous (IV) dose in C57BL/6N mice was evaluated. Nine female C57BL/6N mice with a weight range of 20.4-22.19 grams were administered a 10 mg/kg single IV bolus dose of anti-IL-13/IL-17 bispecific antibody via the tail vein. At the following timepoints post-dose, blood was collected from n=3 mice/timepoint and processed to serum: 5 minutes; 2, 8 and 24 hours; 3, 7, 10, 14 and 21 days. The concentration of anti-IL-13/IL-17 bispecific antibody in each serum sample was determined by ELISA, as described below. Group anti-IL-13/IL-17 bispecific antibody serum concentration versus time profiles were used to evaluate pharmacokinetics using Phoenix WinNonlin v6 (Pharsight; Mountain View, Calif.) using naïve-pooled approach and Non Compartmental Analysis (NCA). The following PK parameters were determined:
Cₘₐₓ: Maximum observed serum concentration
AUCₗₐₛₜ: Area Under the Serum Concentration-Time Curve from day 0 to the last timepoint with measurable anti-IL-13/IL-17 bispecific antibody serum concentration.
CL: Clearance

The concentration of anti-IL-13/IL-17 bispecific antibody in each mouse PK serum sample was determined by a sandwich ELISA for human IgG. In this assay, sheep anti-human IgG (H+L) (Catalog AU003CUS01, Binding site; Birmingham, UK) and goat anti-human IgG (H+L) HRP (Catalog A80-319P-12, Bethyl; Montgomery, Tex.) were used as capture and detection antibodies, respectively. The same lot of anti-IL-13/IL-17 bispecific antibody for dosing animals was used as the standard. The human IgG ELISA tolerated up to 10% C57BL/6 mouse serum matrix with an assay range of 0.39-25 ng/mL. The minimum quantifiable concentration was determined to be 0.039 μg/mL in neat mouse serum (accounting for a minimum sample dilution of 1/100).

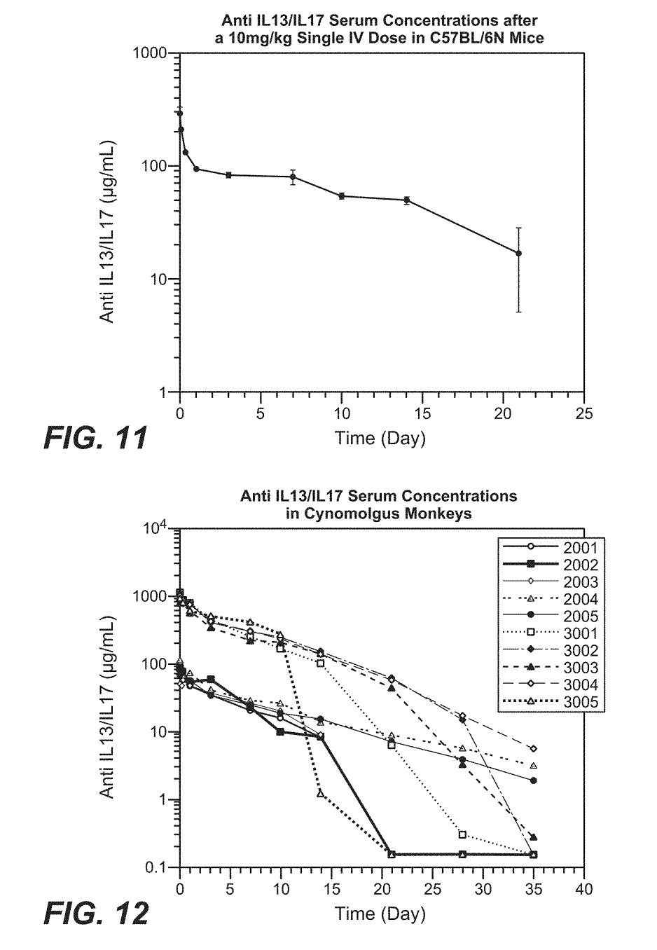

The results of that experiment are shown in FIG. 11. Since a pooled approach was used to evaluate PK, only a single PK parameter estimate is shown in FIG. 11. Following a single IV dose in C57BL/6N mice, anti-IL-13/IL-17 displayed a rapid drop in serum concentration within the first 24 hours post dose, followed by a gradual decrease over the next 20 days. Cₘₐₓ was 211 μg/mL and Area Under the Serum Concentration-Time Curve (AUCₗₐₛₜ) was 1610 day×μg/mL. Clearance (CL) was 6.2 mL/day/kg.

### Example 11

### Pharmacokinetics in Cynomolgus Monkeys

The pharmacokinetics of anti-IL-13/IL-17 bispecific antibody after a single intravenous (IV) dose to cynomolgus monkeys was evaluated. Fifteen male cynomolgus monkeys with a weight range of 2.8-3.7 kg were divided into 3 groups (n=5/group). All animals in each group were administered a single IV dose of vehicle control (Group 1), or 3 or 30 mg/kg of anti-IL-13/IL-17 bispecific antibody (Groups 2 and 3, respectively). At the following timepoints post-dose, blood was collected and processed to serum: predose; 15 minutes; 1, 2, 4, 8 and 24 hours; 3, 7, 10, 14 and 21 days. The concentration of anti-IL-13/IL-17 bispecific antibody in each serum sample was determined by ELISA, as described below. Serum was also tested for the presence of Anti-Therapeutic Antibodies (ATA), as described below. Anti-IL-13/IL-17 bispecific antibody serum concentration versus time profiles for individual animals were used to evaluate pharmacokinetics using Phoenix WinNonlin v6 (Pharsight; Mountain View, Calif.) and individual and group mean pharmacokinetic parameters were reported. The following PK parameters were determined:
Cₘₐₓ: Maximum observed serum concentration
AUCₗₐₛₜ: Area Under the Serum Concentration-Time Curve from day 0 to the last timepoint with measurable anti-IL-13/IL-17 serum concentration.
CL: Clearance
Vₛₛ: Volume of distribution at Steady State

The concentration of anti-IL-13/IL-17 bispecific antibody in individual cynomolgus monkey serum samples was analyzed by sandwich human IgG ELISA, as described above for the mouse pharmacokinetic study. The human IgG ELISA tolerated up to 5% cynomolgus monkey serum matrix with an assay range of 0.39-25 ng/mL. The minimum quantifiable concentration was determined to be 0.039 μg/mL in neat cynomolgus monkey serum (accounting for a minimum sample dilution of 1/100).

Anti-therapeutic antibodies (ATAs) in cynomolgus monkey serum samples were detected using a homogenous bridging ELISA, in which ATAs were allowed to bridge biotinylated anti-IL-13/IL-17 bispecific antibody and digoxigenin (DIG)-labeled anti-IL-13/IL-17 bispecific antibody. Biotin-anti-IL-13/IL-17 bispecific antibody and DIG-anti-IL-13/IL-17 bispecific antibody were prepared in house using EZ-link sulfo-NHS-Lc-biotin (Catalog 21327, Pierce; Rochester, N.Y.) and 3-amino-3-deoxydigoxigenin hemisuccinimide, succinimidyl ester (Catalog A2952, Invitrogen; Carlsbad, Calif.), respectively, according to the manufacturer's protocols. The samples were first incubated with biotin-anti-IL-13/IL-17 bispecific antibody and DIG-anti-IL-13/IL-17 bispecific antibody at 4° C. overnight. The anti-IL-13/IL-17 bispecific antibody—ATA immune complexes were then captured on Nunc Maxisorp 384-well plates pre-coated with 2 μg/mL of Neutravidin (Thermo Scientific; Rockford, Ill.) and detected using horseradish peroxidase (HRP)-labeled mouse anti-DIG antibody (Jackson Immunoresearch; West Grove, Pa.). After incubation with substrate 3,3′,5,5′-tetramethyl benzidine (TMBE-1000, Moss; Pasadena, Md.), the absorbance (optical density, OD) of each sample well was obtained. Samples with an OD equal to or greater than the assay cutpoint (described below) were considered ATA positive. The assay tolerated up to 2% of serum matrix, and was capable to detect 0.6 μg/mL ATA in neat serum with the presence of 50 μg/mL of anti-IL-13/IL-17 bispecific antibody.

To define the assay cutpoint, the individual OD was obtained for a panel of 32 drug-naïve cynomolgus monkey serum samples (Bioreclamation; Westbury, N.Y.). In addition, the OD of the negative control (pooled naïve cynomolgus monkey sera, Bioreclamation) was obtained from the same assay plate. Each animal was normalized by dividing the individual OD with the OD of the negative control to give individual OD ratios and the mean value of the ratios was determined. The cutpoint factor was calculated as this value plus 1.65 times its standard deviation. Three separate experiments were run and the assay cutpoint factor was determined to be 1.9 by averaging the results. When analyzing samples, the cutpoint of each assay plate was determined by multiplying this cutpoint factor by the mean OD of the negative control on the same plate. The cutpoint calculated for this assay gave an estimated false-positive rate of approximately 5%.

The results of the experiment are shown in FIG. 12 and Table 8. Table 8 shows the dose administered to each animal shown in FIG. 12.

TABLE 8
Pharmacokinetic values for cynomolgus monkeys administered
anti-IL-13/IL-17 bispecific antibody

| | Cmax | AUClast | CL | Vss |
|---|---|---|---|---|
| Animal/Group | (μg/mL) | (day × μg/mL) | (mL/day/kg) | (mL/kg) |
| 2001-3 mg/kg | 89.1 | 396 | 7.56 | 36.6 |
| 2002-3 mg/kg | 89.6 | 460 | 6.52 | 31.1 |
| 2003-3 mg/kg | 61.1 | 429 | 6.98 | 35.6 |
| 2004-3 mg/kg | 115 | 663 | 4.26 | 54.0 |
| 2005-3 mg/kg | 94.6 | 546 | 5.30 | 58.1 |
| Mean | 89.9 | 499 | 6.12 | 43.1 |
| SD | 19.3 | 107 | 1.33 | 12.1 |
| 3001-30 mg/kg | 1110 | 4970 | 6.03 | 23.6 |
| 3002-30 mg/kg | 1090 | 6020 | 4.98 | 25.1 |
| 3003-30 mg/kg | 903 | 4990 | 6.01 | 16.6 |
| 3004-30 mg/kg | 988 | 6180 | 4.82 | 33.3 |
| 3005-30 mg/kg | 1160 | 5320 | 5.64 | 18.6 |
| Mean | 1050 | 5500 | 5.50 | 23.4 |
| SD | 102 | 571 | 0.567 | 6.51 |

Anti-IL-13/IL-17 bispecific antibody serum concentrations for animals treated with vehicle control (Group 1) were found to be Less Than Reportable (LTR), therefore, no pharmacokinetic analysis was conducted for these animals.

The Group mean Cmax was 89.9±107 and 1050±102 μg/mL for Groups 2 and 3, respectively. Group mean AUCₗₐₛₜ was 499±107 and 5500±571 day*μg/mL for Groups 2 and 3, respectively. Both Cmax and AUCₗₐₛₜ were observed to increase proportionally with dose. Group mean clearance (CL) was 6.12±1.33 and 5.50±0.567 mL/day/kg for Groups 2 and 3, respectively. Group mean Vss was 43.1±12.1 and 23.4±6.51 for Groups 2 and 3, respectively.

Of the 10 animals treated with anti-IL-13/IL-17 bispecific antibody, eight tested positive for anti-therapeutic antibodies (3/5 animals in group 2 and 5/5 animals in group 3).

### Example 12

### IL-17AA Target Engagement in Cynomolgus Monkeys

The cynomolgus monkey PK study is described in Example 11, above. An ELISA was developed to quantify total IL-17AA in cynomolgus monkey serum to demonstrate target engagement. Antibodies specific to IL-17AA were used for capture and detection, and in-house generated recombinant cynomolgus monkey IL-17AA was used to prepare assay standards and controls. Serum was collected at day −7 and day 0 for baseline values, and at days 1, 3, 8, 11, 15, 22, 29, and 36 post-dose, and total IL-17AA levels of these samples were determined.

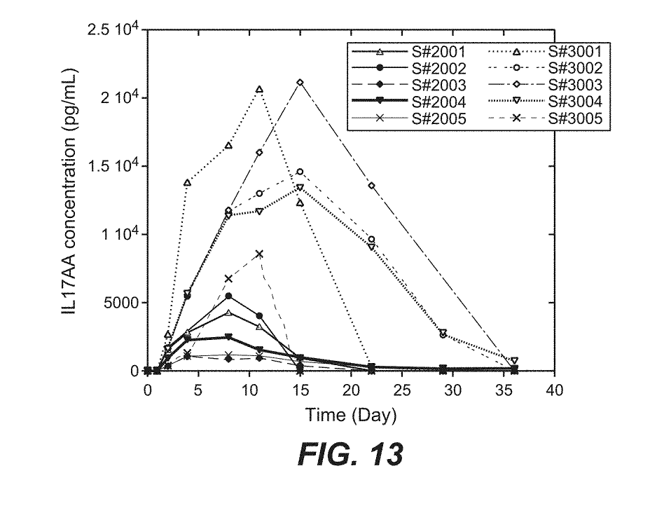

The results are shown in FIG. 13. Free IL-17AA in the serum has a very short half-life typical of a cytokine and is below assay detection limits at baseline. When serum IL-17AA is bound by antibody it will take on the longer half-life of the antibody and become detectable. In the cynomolgus monkey PK study, IL-17AA levels were undetectable at baseline and robust increases in IL-17AA levels were observed after dosing with anti-IL-13/IL-17 bispecific antibody, confirming IL-17AA engagement. The effect was seen with both doses and was dose-dependent. The response was correlated with the pharmacokinetic results described above.

### Example 13

### Anti-IL-17, Anti-IL-13, and Anti-IL-13 Plus Anti-IL-17 Antibody Efficacy in a Mouse House Dust Mite Asthma Model

The mouse house dust mite (HDM) asthma model was performed substantially as described above in Example 4. Biomarker levels were determined in serum and plasma using the following assays according to manufacturer protocol: TARC (R&D, MCC170), CXCL1 (Millipore, MCYTOMAG-70K), and G-CSF (Millipore, MCYTOMAG-70K).

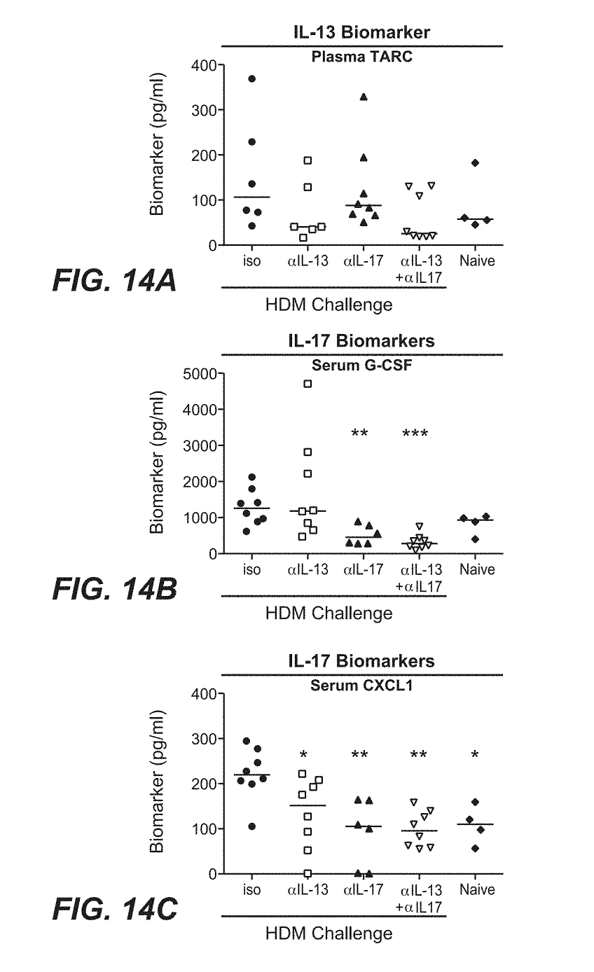

Serum TARC levels were shown to decrease after lebrikizumab treatment in Phase 2 studies. See, e.g., Corren et al., 2011, New England J. Medicine 365:1088-98 and US2012/0156194. As shown in FIG. 14A, in the HDM model, plasma TARC levels trended down after anti-IL-13 antibody or anti-IL-13 antibody plus anti-IL-17 antibody treatments, but not after anti-IL-17 antibody treatment, confirming IL-13 pathway-specific modulation. Similarly, as shown in FIGS. 14B and 9C, IL-17 pathway biomarkers, G-CSF and CXCL1, were significantly reduced in the serum after anti-IL-17 antibody and anti-IL-13 antibody plus anti-IL-17 antibody treatments. In FIG. 14, *p<0.05, **p<0.005, and ***p<0.0005.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of treating an eosinophilic disorder in a patient comprising administering to the patient an effective amount of an anti-IL-13/IL-17 antibody comprising a first half antibody and a second half antibody, wherein the first half antibody comprises a first VH/VL unit that specifically binds to IL-17 and the second half antibody comprises a second VH/VL unit that specifically binds IL-13, wherein the first VH/VL unit comprises HVR-H1 comprising the amino acid sequence of SEQ ID NO: 40, HVR-H2 comprising an amino acid sequence selected from SEQ ID NOs: 43, HVR-H3 comprising the amino acid sequence of SEQ ID NO: 44, HVR-L1 comprising the amino acid sequence of SEQ ID NO: 45, HVR-L2 comprising the amino acid sequence of SEQ ID NO: 46, and HVR-L3 comprising the amino acid sequence of SEQ ID NO: 47, and wherein the second VH/VL unit comprises HVR-H1 comprising the amino acid sequence of SEQ ID NO: 15, HVR-H2 comprising the amino acid sequence of SEQ ID NO: 16, HVR-H3 comprising the amino acid sequence of SEQ ID NO: 17, HVR-L1 comprising the amino acid sequence of SEQ ID NO: 18, HVR-L2 comprising the amino acid sequence of SEQ ID NO: 19, and HVR-L3 comprising the amino acid sequence of SEQ ID NO: 20.

2-60. (canceled)

61. The method of claim 1, wherein the first VH/VL unit comprises the VH sequence having at least 95% sequence identity to the sequence of SEQ ID NO: 39 and the VL sequence having at least 95% sequence identity to the sequence of SEQ ID NO: 38, and the second VH/VL unit comprises the VH sequence having at least 95% sequence identity to the sequence of SEQ ID NO: 13 and the VL sequence having at least 95% sequence identity to the sequence of SEQ ID NO: 14.

62. The method of claim 1, wherein the first VH/VL unit comprises the VH sequence having at least 98% sequence identity to the sequence of SEQ ID NO: 39 and the VL sequence having at least 98% sequence identity to the sequence of SEQ ID NO: 38, and the second VH/VL unit comprises the VH sequence having at least 98% sequence identity to the sequence of SEQ ID NO: 13 and the VL sequence having at least 98% sequence identity to the sequence of SEQ ID NO: 14.

63. The method of claim 1, wherein the first VH/VL unit comprises the VH sequence having at least 99% sequence identity to the sequence of SEQ ID NO: 39 and the VL sequence having at least 99% sequence identity to the sequence of SEQ ID NO: 38, and the second VH/VL unit comprises the VH sequence having at least 99% sequence identity to the sequence of SEQ ID NO: 13 and the VL sequence having at least 99% sequence identity to the sequence of SEQ ID NO: 14.

64. The method of claim 1, wherein the first VH/VL unit comprises the VH sequence of SEQ ID NO: 39 and the VL sequence of SEQ ID NO: 38, and the second VH/VL unit comprises the VH sequence of SEQ ID NO: 13 and the VL sequence of SEQ ID NO: 14.

65. The method of claim 1, wherein the antibody is an IgG antibody.

66. The method of claim 65, wherein the antibody is an IgG1 or IgG4 antibody.

67. The method of claim 66, wherein the antibody is an IgG4 antibody.

68. The method of claim 1 wherein the first half antibody comprises a first heavy chain comprising the sequence of SEQ ID NO: 72 or 117, and a first light chain comprising the sequence of SEQ ID NO: 73, and wherein the second half antibody comprises a second heavy chain comprising the sequence of SEQ ID NO: 21 or 116, and a second light chain comprising the sequence of SEQ ID NO: 22.

69. The method of claim 1, wherein the eosinophilic disorder is asthma.

70. The method of claim 69, wherein the eosinophilic disorder is moderate to severe asthma.

71. The method of claim 69, wherein the asthma is uncontrolled on a corticosteroid.

72. The method of claim 1, further comprising administering to the patient a corticosteroid.

73. The method of claim 72, wherein the corticosteroid is an inhaled corticosteroid.

74. The method of claim 1, wherein the eosinophilic disorder is COPD.

75. The method of claim 1, wherein the eosinophilic disorder is atopic dermatitis.

76. The method of claim 1, wherein the patient has been determined to have a serum periostin level of 20 ng/ml or higher.

77. The method of claim 76, wherein the patient has been determined to have a serum periostin level of 50 ng/ml or higher.

78. The method of claim 76, wherein the serum periostin level is determined by ELISA.

79. The method of claim 76, wherein the serum periostin level is determined by the E4 assay or ELECSYS® periostin assay.

80. The method of claim 76, wherein the serum periostin is Total Periostin.

81. The method of claim 1, wherein the patient has been determined to have a blood eosinophil count of at least 150/μl.

82. The method of claim 1, wherein the patient has been determined to have a blood eosinophil count of at least 200/μl.

83. The method of claim 1, wherein the patient has been determined to have a blood eosinophil count of at least 300/μl.

84. A multispecific antibody comprising a first half antibody and a second half antibody, wherein the first half antibody comprises a first VH/VL unit that specifically binds to IL-17 and the second half antibody comprises a second VH/VL unit that specifically binds IL-13, wherein the first VH/VL unit comprises HVR-H1 comprising the amino acid sequence of SEQ ID NO: 40, HVR-H2 comprising an amino acid sequence selected from SEQ ID NOs: 43, HVR-H3 comprising the amino acid sequence of SEQ ID NO: 44, HVR-L1 comprising the amino acid sequence of SEQ ID NO: 45, HVR-L2 comprising the amino acid sequence of SEQ ID NO: 46, and HVR-L3 comprising the amino acid sequence of SEQ ID NO: 47, and wherein the second VH/VL unit comprises HVR-H1 comprising the amino acid sequence of SEQ ID NO: 15, HVR-H2 comprising the amino acid sequence of SEQ ID NO: 16, HVR-H3 comprising the amino acid sequence of SEQ ID NO: 17, HVR-L1 comprising the amino acid sequence of SEQ ID NO: 18, HVR-L2 comprising the amino acid sequence of SEQ ID NO: 19, and HVR-L3 comprising the amino acid sequence of SEQ ID NO: 20.

85. The multispecific antibody of claim 84, wherein the first VH/VL unit comprises the VH sequence having at least 95% sequence identity to the sequence of SEQ ID NO: 39 and the VL sequence having at least 95% sequence identity to the sequence of SEQ ID NO: 38, and the second VH/VL unit comprises the VH sequence having at least 95% sequence identity to the sequence of SEQ ID NO: 13 and the VL sequence having at least 95% sequence identity to the sequence of SEQ ID NO: 14.

86. The multispecific antibody of claim 84, wherein the first VH/VL unit comprises the VH sequence having at least 98% sequence identity to the sequence of SEQ ID NO: 39 and the VL sequence having at least 98% sequence identity to the sequence of SEQ ID NO: 38, and the second VH/VL unit comprises the VH sequence having at least 98% sequence identity to the sequence of SEQ ID NO: 13 and the VL sequence having at least 98% sequence identity to the sequence of SEQ ID NO: 14.

87. The multispecific antibody of claim 84, wherein the first VH/VL unit comprises the VH sequence having at least 99% sequence identity to the sequence of SEQ ID NO: 39 and the VL sequence having at least 99% sequence identity to the sequence of SEQ ID NO: 38, and the second VH/VL unit comprises the VH sequence having at least 99% sequence identity to the sequence of SEQ ID NO: 13 and the VL sequence having at least 99% sequence identity to the sequence of SEQ ID NO: 14

88. The multispecific antibody of claim 84, wherein the first VH/VL unit comprises the VH sequence of SEQ ID NO: 39 and the VL sequence of SEQ ID NO: 38, and the second VH/VL unit comprises the VH sequence of SEQ ID NO: 13 and the VL sequence of SEQ ID NO: 14.

89. The multispecific antibody of claim 84, wherein the antibody is an IgG antibody.

90. The multispecific antibody of claim 89, wherein the antibody is an IgG1 or IgG4 antibody.

91. The multispecific antibody of claim 90, wherein the antibody is an IgG4 antibody.

92. The multispecific antibody of claim 84 wherein the first half antibody comprises a first heavy chain comprising the sequence of SEQ ID NO: 72 or 117, and a first light chain comprising the sequence of SEQ ID NO: 73, and wherein the second half antibody comprises a second heavy chain comprising the sequence of SEQ ID NO: 21 or 116, and a second light chain comprising the sequence of SEQ ID NO: 22.

93. An isolated nucleic acid encoding the multispecific antibody of claim 84.

94. A host cell comprising the nucleic acid of claim 93.

95. The host cell of claim 94, wherein the host cell is a prokaryotic cell, preferably an E. coli cell.

96. The host cell of claim 94, wherein the host cell is a eukaryotic cell, preferably a CHO cell.

97. The host cell of claim 94, wherein the host cell is an E. coli cell.

98. A method of producing a multispecific antibody comprising culturing the host cell of claim 94 under conditions sufficient to produce the antibody.

99. The method of claim 94, further comprising the step of recovering the multispecific antibody.

100. An isolated nucleic acid comprising (1) the sequence of SEQ ID NO: 107 and the sequence of SEQ ID NO: 108, the sequence of SEQ ID NO:105 and the sequence of SEQ ID NO:106, (2) the sequence of SEQ ID NO:103 and the sequence of SEQ ID NO:104, the sequence of SEQ ID NO:99 and the sequence of SEQ ID NO:100, or (3) the sequence of SEQ ID NO:103, the sequence of SEQ ID NO:104, the sequence of SEQ ID NO:101 and the sequence of SEQ ID NO:102.

101. A host cell comprising the nucleic acid of claim 100.

102. The host cell of claim 101, wherein the host cell is a prokaryotic cell preferably an E. coli cell.

103. The host cell of claim 101, wherein the host cell is a eukaryotic cell preferably a CHO cell.

104. A method of producing a half antibody or a multispecific antibody comprising culturing the host cell of claim 94 under conditions sufficient to produce the half antibody or multispecific antibody.

105. A method of producing a half antibody or a multispecific antibody comprising culturing the host cell of claim 101 under conditions sufficient to produce the half antibody or multispecific antibody.

106. The method of claim 104, further comprising recovering the half antibody or multispecific antibody.

107. The method of claim 105, further comprising recovering the half antibody or multispecific antibody.

108. A method of producing a multispecific antibody comprising (i) culturing a host cell comprising a first nucleic acid comprising the sequence of SEQ ID NO: 99, 101 or 105 and a second nucleic acid comprising the sequence of SEQ ID NO: 100, 102 or 106 wherein the first nucleic acid and the second nucleic acid are comprised on the same nucleic acid molecule or on different nucleic acid molecules, under conditions sufficient to produce a first half antibody, and (ii) culturing a second host cell comprising a first nucleic acid comprising the sequence of SEQ ID NO: 103 or 107 and a second nucleic acid comprising the sequence of SEQ ID NO: 104 or 108, wherein the first nucleic acid and the second nucleic acid are comprised on the same nucleic acid molecule or on different nucleic acid molecules, under conditions sufficient to produce a second half antibody.

109. The method of claim 108, comprising recovering the first half antibody and recovering the second half antibody.

110. The method of claim 109, comprising forming a mixture comprising the first half antibody and the second half antibody under conditions sufficient to produce a multispecific antibody.

111. The method of claim 110, further comprising the step of recovering the multispecific antibody.

112. A multispecific antibody produced by the method of claim 99.

113. A multispecific antibody produced by the method of claim 111.

114. An immunoconjugate comprising the antibody of claim 1 and a cytotoxic agent.

115. A pharmaceutical formulation comprising the antibody of claim 1 and a pharmaceutically acceptable carrier.


SEQUENCE LISTING (124 sequences)

SEQ ID NO: 1 - protein, 132 aa, Homo sapiens
MALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 2 - protein, 114 aa, Homo sapiens
SPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 3 - protein, 122 aa, Homo sapiens
LTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 4 - protein, 132 aa, Macaca fascicularis
MALLLTMVIALTCLGGFASPSPVPPSTALKELIEELVNITQNQKAPLCNGSMVWSINLTAGVYCAALESLINVSGCSAIEKTQRMLNGFCPHKVSAGQFSSLRVRDTKIEVAQFVKDLLVHLKKLFREGQFN

SEQ ID NO: 5 - protein, 8 aa, Homo sapiens
ESLINVSG

SEQ ID NO: 6 - protein, 13 aa, Homo sapiens
YCAALESLINVSG

SEQ ID NO: 7 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 8 - protein, 132 aa, Homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 9 - protein, 163 aa, Homo sapiens
MTVKTLHGPAMVKYLLLSILGLAFLSEAAARKIPKVGHTFFQKPESCPPVPGGSMKLDIGIINENQRVSMSRNIESRSTSPWNYTVTWDPNRYPSEVVQAQCRNLGCINAQGKEDISMNSVPIQQETLVVRRKHQGCSVSFQLEKVLVTVGCTCVTPVIHHVQ

SEQ ID NO: 10 - protein, 133 aa, Homo sapiens [sequence omitted]

SEQ ID NO: 11 - protein, 118 aa, Artificial Sequence [sequence omitted]

SEQ ID NO: 12 - protein, 112 aa, Artificial Sequence
  source: /note=“Description of Artificial Sequence: Synthetic polypeptide”
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKR

SEQ ID NO: 13 - protein, 118 aa, Artificial Sequence
  source: /note=“Description of Artificial Sequence: Synthetic polypeptide”
EVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSS

SEQ ID NO: 14 - protein, 112 aa, Artificial Sequence
  source: /note=“Description of Artificial Sequence: Synthetic polypeptide”
DIVLTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKR

SEQ ID NO: 15 - protein, 5 aa, Artificial Sequence
  source: /note=“Description of Artificial Sequence: Synthetic peptide”
AYSVN

SEQ ID NO: 16 - protein, 16 aa, Artificial Sequence
  source: /note=“Description of Artificial Sequence: Synthetic peptide”
MIWGDGKIVYNSALKS

SEQ ID NO: 17 - protein, 10 aa, Artificial Sequence
  source: /note=“Description of Artificial Sequence: Synthetic peptide”
DGYYPYAMDN

SEQ ID NO: 18 - protein, 15 aa, Artificial Sequence
  source: /note=“Description of Artificial Sequence: Synthetic peptide”
RASKSVDSYGNSFMH

SEQ ID NO: 19 - protein, 7 aa, Artificial Sequence
  source: /note=“Description of Artificial Sequence: Synthetic peptide”
LASNLES

SEQ ID NO: 20 - protein, 9 aa, Artificial Sequence
  source: /note=“Description of Artificial Sequence: Synthetic peptide”
QQNNEDPRT

SEQ ID NO: 21 - protein, 445 aa, Artificial Sequence
  source: /note=“Description of Artificial Sequence: Synthetic polypeptide”
EVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 218 aa, Artificial Sequence
  source: /note=“Description of Artificial Sequence: Synthetic polypeptide”
DIVLTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 445 aa, Artificial Sequence
  source: /note=“Description of Artificial Sequence: Synthetic polypeptide”
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 218 aa, Artificial Sequence
  source: /note=“Description of Artificial Sequence: Synthetic polypeptide”
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 106 aa, Artificial Sequence
  source: /note=“Description of Artificial Sequence: Synthetic polypeptide”
DIVLTQSPASLAVSLGQRATISCRASQSVSTSSYSYMNWYQQTPGQPPKLLIKYASNLESGIPARFSGSGSGTDFTLNIHPVEEEDTATYYCQHSWEIPYTFGGGT

SEQ ID NO: 26 - protein, 123 aa, Artificial Sequence
  source: /note=“Description of Artificial Sequence: Synthetic polypeptide”
QVTLKESGPGILQPSQTLSLTCSFSGFSLSTSDMGVGWIRQPSGKGLEWLAHIWWDDVKRYNPALKSRLTISKDTSSSQVFLKIASVDTADTATYYCARIGTNYGYDGLFDYWGQGTTLTVSS

SEQ ID NO: 27 - protein, 218 aa, Artificial Sequence
  source: /note=“Description of Artificial Sequence: Synthetic polypeptide”
DIVMTQSPDSLAVSLGERATINCRASQSVSTSSYSYMNWYQQKPGQPPKLLIKYASNLESGVPDRFSGSGSGTDFTLTISSLQAEDVAVYYCQHSWEIPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 453 aa, Artificial Sequence
  source: /note=“Description of Artificial Sequence: Synthetic polypeptide”
EVQLVESGPALVKPTQTLTLTCTFSGFSLSTSDMGVGWIRQPPGKALEWLAHIWWDDVKRYNPALKSRLTISKDTSKNQVVLTMTNMDPVDTATYYCARIGTNYGYDALFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 111 aa, Artificial Sequence
  source: /note=“Description of Artificial Sequence: Synthetic polypeptide”
DIVMTQSPDSLAVSLGERATINCRASQSVSTSSYSYMNWYQQKPGQPPKLLIKYASNLESGVPDRFSGSGSGTDFTLTISSLQAEDVAVYYCQHSWEIPYTFGQGTKVEIK

SEQ ID NO: 30 - protein, 123 aa, Artificial Sequence
  source: /note=“Description of Artificial Sequence: Synthetic polypeptide”
EVQLVESGPALVKPTQTLTLTCTFSGFSLSTSDMGVGWIRQPPGKALEWLAHIWWDDVKRYNPALKSRLTISKDTSKNQVVLTMTNMDPVDTATYYCARIGTNYGYDALFDYWGQGTLVTVSS

SEQ ID NO: 31 - protein, 12 aa, Artificial Sequence
  source: /note=“Description of Artificial Sequence: Synthetic peptide”
GFSLSTSDMGVG

SEQ ID NO: 32 - protein, 17 aa, Artificial Sequence
  source: /note=“Description of Artificial Sequence: Synthetic peptide”
AHIWWDDVKRYNPALKS

SEQ ID NO: 33 - protein, 15 aa, Artificial Sequence
  source: /note=“Description of Artificial Sequence: Synthetic peptide”
ARIGTNYGYDALFDY

SEQ ID NO: 34 - protein, 15 aa, Artificial Sequence
  source: /note=“Description of Artificial Sequence: Synthetic peptide”
RASQSVSTSSYSYMN

SEQ ID NO: 35 - protein, 7 aa, Artificial Sequence
  source: /note=“Description of Artificial Sequence: Synthetic peptide”
YASNLES

SEQ ID NO: 36 - protein, 9 aa, Artificial Sequence
  source: /note=“Description of Artificial Sequence: Synthetic peptide”
QHSWEIPYT

SEQ ID NO: 37 - protein, 123 aa, Artificial Sequence
  source: /note=“Description of Artificial Sequence: Synthetic polypeptide”
EVQLVESGGGLVQPGRSLRLSCAASGFTFDDYAMHWVRQAPGKGLEWVSGINWNSGGIGYADSVKGRFTISRDNAKNSLYLQMNSLRAEDTALYYCARDMGGFGEFYWNFGLWGRGTLVTVSS

SEQ ID NO: 38 - protein, 108 aa, Artificial Sequence
  source: /note=“Description of Artificial Sequence: Synthetic polypeptide”
EIVLTQSPATLSLSPGERATLSCRASQSVRSYLAWYQQKPGQAPRLLIYDASNRATGIPARFSGSGSGTDFTLTISSLEPEDFAVYYCQQRSNWPPATFGGGTKVEIK

SEQ ID NO: 39 - protein, 123 aa, Artificial Sequence
  source: /note=“Description of Artificial Sequence: Synthetic polypeptide”
EVQLVESGGGLVQPGRSLRLSCAASGFTFDDYAMHWVRQAPGKGLEWVSGINWSSGGIGYADSVKGRFTISRDNAKNSLYLQMNSLRAEDTALYYCARDIGGFGEFYWNFGLWGRGTLVTVSS

SEQ ID NO: 40 - protein, 5 aa, Artificial Sequence
  source: /note=“Description of Artificial Sequence: Synthetic peptide”
DYAMH

SEQ ID NO: 41 - protein, 17 aa, Artificial Sequence
  source: /note=“Description of Artificial Sequence: Synthetic peptide”
GINWNSGGIGYADSVKG

SEQ ID NO: 42 - protein, 14 aa, Artificial Sequence
  source: /note=“Description of Artificial Sequence: Synthetic peptide”
DMGGFGEFYWNFGL

SEQ ID NO: 43 - protein, 17 aa, Artificial Sequence
  source: /note=“Description of Artificial Sequence: Synthetic peptide”
GINWSSGGIGYADSVKG

SEQ ID NO: 44 - protein, 14 aa, Artificial Sequence
  source: /note=“Description of Artificial Sequence: Synthetic peptide”
DIGGFGEFYWNFGL

SEQ ID NO: 45 - protein, 11 aa, Artificial Sequence
  source: /note=“Description of Artificial Sequence: Synthetic peptide”
RASQSVRSYLA

SEQ ID NO: 46 - protein, 7 aa, Artificial Sequence
  source: /note=“Description of Artificial Sequence: Synthetic peptide”
DASNRAT

SEQ ID NO: 47 - protein, 10 aa, Artificial Sequence
  source: /note=“Description of Artificial Sequence: Synthetic peptide”
QQRSNWPPAT

SEQ ID NO: 48 - protein, 126 aa, Artificial Sequence
  source: /note=“Description of Artificial Sequence: Synthetic polypeptide”
QVQLVQSGAEVKKPGASVKVSCKASGYTFTSYDINWVRQATGQGLEWMGWMNPDSGVIRYAQKFQGRVTMTRNTSISTAYMELNSLRSEDTAVYYCAREWFGELPSYYFYSGMDVWGQGTTVTVSS

SEQ ID NO: 49 - protein, 107 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGPGTKVDIK

SEQ ID NO: 50 - protein, 126 aa, Artificial Sequence
  source: /note=“Description of Artificial Sequence: Synthetic polypeptide”
QVQLVQSGAEVKKPGASVKVSCKASGYTFTSYDINWVRQATGQGLEWMGWLNPDSGVIRYAQKFQGRVTMTRDTSISTAYMELSSLRSEDTAVYYCAREWFGELPSYYFYSGMDVWGQGTTVTVSS

SEQ ID NO: 51 - protein, 5 aa, Artificial Sequence
  source: /note=“Description of Artificial Sequence: Synthetic peptide”
SYDIN

SEQ ID NO: 52 - protein, 17 aa, Artificial Sequence
  source: /note=“Description of Artificial Sequence: Synthetic peptide”
WMNPDSGVIRYAQKFQG

SEQ ID NO: 53 - protein, 17 aa, Artificial Sequence
  source: /note=“Description of Artificial Sequence: Synthetic peptide”
WLNPDSGVIRYAQKFQG

SEQ ID NO: 54 - protein, 17 aa, Artificial Sequence
  source: /note=“Description of Artificial Sequence: Synthetic peptide”
EWFGELPSYYFYSGMDV

SEQ ID NO: 55 - protein, 11 aa, Artificial Sequence
  source: /note=“Description of Artificial Sequence: Synthetic peptide”
RASQSVSSYLA

SEQ ID NO: 56 - protein, 7 aa, Artificial Sequence
  source: /note=“Description of Artificial Sequence: Synthetic peptide”
DASNRAT

SEQ ID NO: 57 - protein, 9 aa, Artificial Sequence
  source: /note=“Description of Artificial Sequence: Synthetic peptide”
QQRSNWPPT

SEQ ID NO: 58 - protein, 127 aa, Artificial Sequence
  source: /note=“Description of Artificial Sequence: Synthetic polypeptide”
QVQLVQSGAEVKKPGSSVKVSCKASGGTLSSYAFSWVRQAPGQGLEWMGGIIPFFGTTNYAQKFQGRVIITADESTNTAYMELSGLRSEDTAVYYCARDRDYYGLGSPFYYYGMDVWGQGTTVTVSS

SEQ ID NO: 59 - protein, 107 aa, Artificial Sequence
  source: /note=“Description of Artificial Sequence: Synthetic polypeptide”
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPWTFGQGTKVEIK

SEQ ID NO: 60 - protein, 107 aa, Artificial Sequence
  source: /note=“Description of Artificial Sequence: Synthetic polypeptide”
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLPWTFGQGTKVEIK

SEQ ID NO: 61 - protein, 5 aa, Artificial Sequence
  source: /note=“Description of Artificial Sequence: Synthetic peptide”
SYAFS

SEQ ID NO: 62 - protein, 17 aa, Artificial Sequence
  source: /note=“Description of Artificial Sequence: Synthetic peptide”
GIIPFFGTTNYAQKFQG

SEQ ID NO: 63 - protein, 18 aa, Artificial Sequence
  source: /note=“Description of Artificial Sequence: Synthetic peptide”
DRDYYGLGSPFYYYGMDV

SEQ ID NO: 64 - protein, 11 aa, Artificial Sequence
  source: /note=“Description of Artificial Sequence: Synthetic peptide”
RASQSIGSSLH

SEQ ID NO: 65 - protein, 7 aa, Artificial Sequence
  source: /note=“Description of Artificial Sequence: Synthetic peptide”
YASQSFS

SEQ ID NO: 66 - protein, 9 aa, Artificial Sequence
  source: /note=“Description of Artificial Sequence: Synthetic peptide”
HQSSSLPWT

SEQ ID NO: 67 - protein, 330 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 330 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 69 - protein, 327 aa, Artificial Sequence
  source: /note=“Description of Artificial Sequence: Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 70 - protein, 327 aa, Artificial Sequence
  source: /note=“Description of Artificial Sequence: Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 71 - protein, 450 aa, Artificial Sequence
  source: /note=“Description of Artificial Sequence: Synthetic polypeptide”
EVQLVESGGGLVQPGRSLRLSCAASGFTFDDYAMHWVRQAPGKGLEWVSGINWNSGGIGYADSVKGRFTISRDNAKNSLYLQMNSLRAEDTALYYCARDMGGFGEFYWNFGLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 72 - protein, 450 aa, Artificial Sequence
  source: /note=“Description of Artificial Sequence: Synthetic polypeptide”
EVQLVESGGGLVQPGRSLRLSCAASGFTFDDYAMHWVRQAPGKGLEWVSGINWSSGGIGYADSVKGRFTISRDNAKNSLYLQMNSLRAEDTALYYCARDIGGFGEFYWNFGLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 73 - protein, 215 aa, Artificial Sequence
  source: /note=“Description of Artificial Sequence: Synthetic polypeptide”
EIVLTQSPATLSLSPGERATLSCRASQSVRSYLAWYQQKPGQAPRLLIYDASNRATGIPARFSGSGSGTDFTLTISSLEPEDFAVYYCQQRSNWPPATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 453 aa, Artificial Sequence
  source: /note=“Description of Artificial Sequence: Synthetic polypeptide”
QVQLVQSGAEVKKPGASVKVSCKASGYTFTSYDINWVRQATGQGLEWMGWMNPDSGVIRYAQKFQGRVTMTRNTSISTAYMELNSLRSEDTAVYYCAREWFGELPSYYFYSGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 75 - protein, 453 aa, Artificial Sequence
  source: /note=“Description of Artificial Sequence: Synthetic polypeptide”
QVQLVQSGAEVKKPGASVKVSCKASGYTFTSYDINWVRQATGQGLEWMGWLNPDSGVIRYAQKFQGRVTMTRDTSISTAYMELSSLRSEDTAVYYCAREWFGELPSYYFYSGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 76 - protein, 214 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 454 aa, Artificial Sequence
  source: /note=“Description of Artificial Sequence: Synthetic polypeptide”
QVQLVQSGAEVKKPGSSVKVSCKASGGTLSSYAFSWVRQAPGQGLEWMGGIIPFFGTTNYAQKFQGRVIITADESTNTAYMELSGLRSEDTAVYYCARDRDYYGLGSPFYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 78 - protein, 214 aa, Artificial Sequence
  source: /note=“Description of Artificial Sequence: Synthetic polypeptide”
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 214 aa, Artificial Sequence
  source: /note=“Description of Artificial Sequence: Synthetic polypeptide”
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 17 aa, Artificial Sequence
  source: /note=“Description of Artificial Sequence: Synthetic peptide”
  MOD_RES (3)..(3): Any amino acid except Asn
GIXWSSGGIGYADSVKG

SEQ ID NO: 81 - protein, 17 aa, Artificial Sequence
  source: /note=“Description of Artificial Sequence: Synthetic peptide”
  MOD_RES (5)..(5): Any amino acid except Ser or Thr
GINWXSGGIGYADSVKG

SEQ ID NO: 82 - protein, 123 aa, Artificial Sequence
  source: /note=“Description of Artificial Sequence: Synthetic polypeptide”
  MOD_RES (52)..(52): Any amino acid except Asn
EVQLVESGGGLVQPGRSLRLSCAASGFTFDDYAMHWVRQAPGKGLEWVSGIXWSSGGIGYADSVKGRFTISRDNAKNSLYLQMNSLRAEDTALYYCARDIGGFGEFYWNFGLWGRGTLVTVSS

SEQ ID NO: 83 - protein, 123 aa, Artificial Sequence
  source: /note=“Description of Artificial Sequence: Synthetic polypeptide”
  MOD_RES (54)..(54): Any amino acid except Ser or Thr
EVQLVESGGGLVQPGRSLRLSCAASGFTFDDYAMHWVRQAPGKGLEWVSGINWXSGGIGYADSVKGRFTISRDNAKNSLYLQMNSLRAEDTALYYCARDIGGFGEFYWNFGLWGRGTLVTVSS

SEQ ID NO: 84 - protein, 450 aa, Artificial Sequence
  source: /note=“Description of Artificial Sequence: Synthetic polypeptide”
  MOD_RES (52)..(52): Any amino acid except Asn
EVQLVESGGGLVQPGRSLRLSCAASGFTFDDYAMHWVRQAPGKGLEWVSGIXWSSGGIGYADSVKGRFTISRDNAKNSLYLQMNSLRAEDTALYYCARDIGGFGEFYWNFGLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 85 - protein, 450 aa, Artificial Sequence
  source: /note=“Description of Artificial Sequence: Synthetic polypeptide”
  MOD_RES (54)..(54): Any amino acid except Ser or Thr
EVQLVESGGGLVQPGRSLRLSCAASGFTFDDYAMHWVRQAPGKGLEWVSGINWXSGGIGYADSVKGRFTISRDNAKNSLYLQMNSLRAEDTALYYCARDIGGFGEFYWNFGLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 86 - protein, 866 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 87 - protein, 833 aa, Homo sapiens
RLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 88 - protein, 791 aa, Homo sapiens
MPVPWFLLSLALGRSPVVLSLERLVGPQDATHCSPVSLEPWGDEERLRVQFLAQQSLSLAPVTAATARTALSGLSGADGRREERGRGKSWVCLSLGGSGNTEPQKKGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 89 - protein, 771 aa, Homo sapiens
LERLVGPQDATHCSPVSLEPWGDEERLRVQFLAQQSLSLAPVTAATARTALSGLSGADGRREERGRGKSWVCLSLGGSGNTEPQKKGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 90 - protein, 427 aa, Homo sapiens
MEWPARLCGLWALLLCAGGGGGGGGAAPTETQPPVTNLSVSVENLCTVIWTWNPPEGASSNCSLWYFSHFGDKQDKKIAPETRRSIEVPLNERICLQVGSQCSTNESEKPSILVEKCISPPEGDPESAVTELQCIWHNLSYMKCSWLPGRNTSPDTNYTLYYWHRSLEKIHQCENIFREGQYFGCSFDLTKVKDSSFEQHSVQIMVKDNAGKIKPSFNIVPLTSRVKPDPPHIKNLSFHNDDLYVQWENPQNFISRCLFYEVEVNNSQTETHNVFYVQEAKCENPEFERNVENTSCFMVPGVLPDTLNTVRIRVKTNKLCYEDDKLWSNWSQEMSIGKKRNSTLYITMLLIVPVIVAGAIIVLLLYLKRLKIIIFPPIPDPGKIFKEMFGDQNDDTLHWKKYDIYEKQTKEETDSVVLIENLKKASQ

SEQ ID NO: 91 - protein, 406 aa, Homo sapiens
GGGGAAPTETQPPVTNLSVSVENLCTVIWTWNPPEGASSNCSLWYFSHFGDKQDKKIAPETRRSIEVPLNERICLQVGSQCSTNESEKPSILVEKCISPPEGDPESAVTELQCIWHNLSYMKCSWLPGRNTSPDTNYTLYYWHRSLEKIHQCENIFREGQYFGCSFDLTKVKDSSFEQHSVQIMVKDNAGKIKPSFNIVPLTSRVKPDPPHIKNLSFHNDDLYVQWENPQNFISRCLFYEVEVNNSQTETHNVFYVQEAKCENPEFERNVENTSCFMVPGVLPDTLNTVRIRVKTNKLCYEDDKLWSNWSQEMSIGKKRNSTLYITMLLIVPVIVAGAIIVLLLYLKRLKIIIFPPIPDPGKIFKEMFGDQNDDTLHWKKYDIYEKQTKEETDSVVLIENLKKASQ

SEQ ID NO: 92 - protein, 825 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 93 - protein, 800 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 94 - protein, 132 aa, Macaca fascicularis
GIAIPRNSGCPNSEDKNFPRTVMVNLNIHNRNTSTNPKRSSDYYNRSTSPWNLHRNEDPERYPSVIWEAKCRHLGCVKADGNVDYHMNSVPIQQEILVLRREPRHCPNSFRLEKILVSVGCTCVTPIVHHVA

SEQ ID NO: 95 - protein, 133 aa, Macaca fascicularis
RKIPKVGHTFFQKPESCPPVPEGSMKLDTGIINENQRVSMSRNIESRSTSPWNYTVTWDPNRYPSEVVQAQCKHLGCINAQGKEDISMNSVPIQQETLVLRRKHQGCSVSFQLEKVLVTVGCTCVTPVVHHVQ

SEQ ID NO: 96 - DNA, 19 bp, Artificial Sequence
  source: /note=“Description of Artificial Sequence: Synthetic primer”
agcggattctcatggaaca

SEQ ID NO: 97 - DNA, 18 bp, Artificial Sequence
  source: /note=“Description of Artificial Sequence: Synthetic primer”
ctggtcagccaggagctt

SEQ ID NO: 98 - DNA, 24 bp, Artificial Sequence
  source: /note=“Description of Artificial Sequence: Synthetic probe”
tccacaagctgaaggcagacaagg

SEQ ID NO: 99 - DNA, 1353 bp, Artificial Sequence
  source: /note=“Description of Artificial Sequence: Synthetic polynucleotide”
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattaattggagcagtggtggcataggctatgcggactctgtgaagggccgattcaccatctccagagacaacgccaagaactccctgtatctgcaaatgaacagtctgagagctgaggacacggccttgtattactgtgcaagagatatcggggggttcggggagttttactggaacttcggtctctggggccgtggcaccctggtcactgtctcctcagcctccaccaagggcccatcggtcttccccctggcaccctgctcccgcagtacttctgagtccacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcaccaagacctacacgtgcaacgtggatcacaagcccagcaacaccaaggtggacaaacgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgagctgcgctgtcaaaggcttctaccccagcgacatcgccgtggagtgggagagcaatgggcagccggagaacaactacaagaccacgcctcccgtgctggactccgacggctccttcttcctcgttagcaggctaaccgtggacaagagcaggtggcaggaggggaatgtcttctcatgctccgtgatgcatgaggctctgcacaaccactacacacagaagagcctctccctgtctctgggtaaataa

SEQ ID NO: 100 - DNA, 648 bp, Artificial Sequence
  source: /note=“Description of Artificial Sequence: Synthetic polynucleotide”
gaaattgtgttgacacagtctccagccaccctgtctttgtctccaggggaaagagccaccctctcctgcagggccagtcagagtgttagaagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggccactttcggcggagggaccaaggtggagatcaaacgaactgtggctgcaccatctgtcttcatcttcccgccatctgatgagcagttgaaatctggaactgcttctgttgtgtgcctgctgaataacttctatccccgtgaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 101 - DNA, 1353 bp, Artificial Sequence
  source: /note=“Description of Artificial Sequence: Synthetic polynucleotide”
gaagttcagctggttgaaagcggtggtggtctggttcagcctggtcgtagcctgcgtctgagctgtgcagcaagcggttttacctttgatgattatgccatgcattgggttcgtcaggcaccgggtaaaggtctggaatgggttagcggtattaattggagcagcggtggtattggttatgcagatagcgttaaaggtcgttttaccattagccgtgataatgccaaaaatagcctgtacctgcagatgaatagtctgcgtgcagaagataccgcactgtattattgtgcacgtgatattggtggttttggcgaattctattggaattttggtctgtggggtcgtggcaccctggttaccgttagcagcgcctccaccaagggcccatcggtcttccccctggcaccctgctcccgcagtacttctgagtccacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcaccaagacctacacgtgcaacgtggatcacaagcccagcaacaccaaggtggacaaacgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgagctgcgctgtcaaaggcttctaccccagcgacatcgccgtggagtgggagagcaatgggcagccggagaacaactacaagaccacgcctcccgtgctggactccgacggctccttcttcctcgttagcaggctaaccgtggacaagagcaggtggcaggaggggaatgtcttctcatgctccgtgatgcatgaggctctgcacaaccactacacacagaagagcctctccctgtctctgggtaaataa

SEQ ID NO: 102 - DNA, 648 bp, Artificial Sequence
  source: /note=“Description of Artificial Sequence: Synthetic polynucleotide”
gaaattgttctgacccagagtccggcaaccctgagcctgagtccgggtgaacgtgccaccctgagctgtcgtgcaagccagagcgttcgtagctatctggcatggtatcagcagaaaccgggtcaggcaccgcgtctgctgatttatgatgcaagcaatcgtgcaaccggtattccggcacgttttagcggtagcggtagtggcaccgattttaccctgaccattagcagcctggaaccggaagattttgcagtgtattattgtcagcagcgtagcaattggccaccggcaacctttggtggtggcaccaaagttgaaattaaacgaactgtggctgcaccatctgtcttcatcttcccgccatctgatgagcagttgaaatctggaactgcttctgttgtgtgcctgctgaataacttctatccccgtgaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 103 - DNA, 1338 bp, Artificial Sequence
  source: /note=“Description of Artificial Sequence: Synthetic polynucleotide”
gaagttaccctgcgcgagagcggcccagccctggtgaagccaacccagaccctgaccctgacctgcaccgtcagcggcttcagcctgagcgcctacagcgtgaactggatccgccagccaccaggcaaggccctggagtggctggccatgatctggggcgacggcaagatcgtgtacaacagcgccctgaagagccgcctgaccatcagcaaggacaccagcaagaaccaggtggtgctgaccatgaccaacatggacccagtggacaccgccacctactactgcgccggcgacggctactacccatacgccatggacaactggggccagggcagcctggtgaccgtgagcagcgcttccaccaagggcccatcggtcttccccctggcaccctgctcccgcagtacttctgagtccacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcaccaagacctacacgtgcaacgtggatcacaagcccagcaacaccaaggtggacaaacgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgtggtgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaataa

SEQ ID NO: 104 - DNA, 657 bp, Artificial Sequence
  source: /note=“Description of Artificial Sequence: Synthetic polynucleotide”
gatatcgtgctgacccagagcccagacagcctgtctgtgagcctgggcgagcgcgccaccatcaactgccgcgccagcaaaagcgtggacagctacggcaacagcttcatgcactggtatcagcagaagccaggccagccacccaagctgctgatctacctggccagcaacctggagagcggcgtgccagaccgcttcagcggcagcggcagcggcaccgacttcaccctgaccatcagctctctgcaggccgaggatgtggccgtgtactactgccagcagaacaacgaggacccacgcaccttcggtggcggtaccaaggtggagatcaaacgaactgtggctgcaccatctgtcttcatcttcccgccatctgatgagcagttgaaatctggaactgcttctgttgtgtgcctgctgaataacttctatccccgtgaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 105 - DNA, 369 bp, Artificial Sequence
  source: /note=“Description of Artificial Sequence: Synthetic polynucleotide”
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattaattggagcagtggtggcataggctatgcggactctgtgaagggccgattcaccatctccagagacaacgccaagaactccctgtatctgcaaatgaacagtctgagagctgaggacacggccttgtattactgtgcaagagatatcggggggttcggggagttttactggaacttcggtctctggggccgtggcaccctggtcactgtctcctca

SEQ ID NO: 106 - DNA, 324 bp, Artificial Sequence
  source: /note=“Description of Artificial Sequence: Synthetic polynucleotide”
gaaattgtgttgacacagtctccagccaccctgtctttgtctccaggggaaagagccaccctctcctgcagggccagtcagagtgttagaagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggccactttcggcggagggaccaaggtggagatcaaa

SEQ ID NO: 107 - DNA, 354 bp, Artificial Sequence
  source: /note=“Description of Artificial Sequence: Synthetic polynucleotide”
gaagttaccctgcgcgagagcggcccagccctggtgaagccaacccagaccctgaccctgacctgcaccgtcagcggcttcagcctgagcgcctacagcgtgaactggatccgccagccaccaggcaaggccctggagtggctggccatgatctggggcgacggcaagatcgtgtacaacagcgccctgaagagccgcctgaccatcagcaaggacaccagcaagaaccaggtggtgctgaccatgaccaacatggacccagtggacaccgccacctactactgcgccggcgacggctactacccatacgccatggacaactggggccagggcagcctggtgaccgtgagcagc

SEQ ID NO: 108 - DNA, 333 bp, Artificial Sequence
  source: /note=“Description of Artificial Sequence: Synthetic polynucleotide”
gatatcgtgctgacccagagcccagacagcctgtctgtgagcctgggcgagcgcgccaccatcaactgccgcgccagcaaaagcgtggacagctacggcaacagcttcatgcactggtatcagcagaagccaggccagccacccaagctgctgatctacctggccagcaacctggagagcggcgtgccagaccgcttcagcggcagcggcagcggcaccgacttcaccctgaccatcagctctctgcaggccgaggatgtggccgtgtactactgccagcagaacaacgaggacccacgcaccttcggtggcggtaccaaggtggagatcaaa

SEQ ID NO: 109 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 110 - protein, 779 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYKPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 111 - protein, 781 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYRPTLTKVKIEGEPEFRLIKEGETITEVIHGEPIIKKYTKIIDGVPVEITEKETREERIITGPEIKYTRISTGGGETEETLKKLLQEDTPVRKLQANKKVQGSRRRLREGRSQ

SEQ ID NO: 112 - protein, 751 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYKPIIKKYTKIIDGVPVEITEKETREERIITGPEIKYTRISTGGGETEETLKKLLQEDTPVRKLQANKKVQGSRRRLREGRSQ

SEQ ID NO: 113 - protein, 695 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYSPEIKYTRISTGGGETEETLKKLLQE

SEQ ID NO: 114 - protein, 17 aa, Artificial Sequence
  source: /note=“Description of Artificial Sequence: Synthetic peptide”
  VARIANT (3)..(3): /replace=“Ser” or “Gln”
  misc_feature (1)..(17): /note=“Variant residues given in the sequence have no preference with respect to those in the annotations for variant positions”
GIDWSSGGIGYADSVKG

SEQ ID NO: 115 - protein, 123 aa, Artificial Sequence
  source: /note=“Description of Artificial Sequence: Synthetic polypeptide”
  VARIANT (52)..(52): /replace=“Ser” or “Gln”
  misc_feature (1)..(123): /note=“Variant residues given in the sequence have no preference with respect to those in the annotations for variant positions”
EVQLVESGGGLVQPGRSLRLSCAASGFTFDDYAMHWVRQAPGKGLEWVSGIDWSSGGIGYADSVKGRFTISRDNAKNSLYLQMNSLRAEDTALYYCARDIGGFGEFYWNFGLWGRGTLVTVSS

SEQ ID NO: 116 - protein, 444 aa, Artificial Sequence
  source: /note=“Description of Artificial Sequence: Synthetic polypeptide”
EVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 117 - protein, 449 aa, Artificial Sequence
  source: /note=“Description of Artificial Sequence: Synthetic polypeptide”
EVQLVESGGGLVQPGRSLRLSCAASGFTFDDYAMHWVRQAPGKGLEWVSGINWSSGGIGYADSVKGRFTISRDNAKNSLYLQMNSLRAEDTALYYCARDIGGFGEFYWNFGLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 118 - DNA, 1332 bp, Artificial Sequence
  source: /note=“Description of Artificial Sequence: Synthetic polynucleotide”
gaagttaccctgcgcgagagcggcccagccctggtgaagccaacccagaccctgaccctgacctgcaccgtcagcggcttcagcctgagcgcctacagcgtgaactggatccgccagccaccaggcaaggccctggagtggctggccatgatctggggcgacggcaagatcgtgtacaacagcgccctgaagagccgcctgaccatcagcaaggacaccagcaagaaccaggtggtgctgaccatgaccaacatggacccagtggacaccgccacctactactgcgccggcgacggctactacccatacgccatggacaactggggccagggcagcctggtgaccgtgagcagcgcttccaccaagggcccatcggtcttccccctggcaccctgctcccgcagtacttctgagtccacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcaccaagacctacacgtgcaacgtggatcacaagcccagcaacaccaaggtggacaaacgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgtggtgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 119 - DNA, 1347 bp, Artificial Sequence
  source: /note=“Description of Artificial Sequence: Synthetic polynucleotide”
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattaattggagcagtggtggcataggctatgcggactctgtgaagggccgattcaccatctccagagacaacgccaagaactccctgtatctgcaaatgaacagtctgagagctgaggacacggccttgtattactgtgcaagagatatcggggggttcggggagttttactggaacttcggtctctggggccgtggcaccctggtcactgtctcctcagcctccaccaagggcccatcggtcttccccctggcaccctgctcccgcagtacttctgagtccacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcaccaagacctacacgtgcaacgtggatcacaagcccagcaacaccaaggtggacaaacgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgagctgcgctgtcaaaggcttctaccccagcgacatcgccgtggagtgggagagcaatgggcagccggagaacaactacaagaccacgcctcccgtgctggactccgacggctccttcttcctcgttagcaggctaaccgtggacaagagcaggtggcaggaggggaatgtcttctcatgctccgtgatgcatgaggctctgcacaaccactacacacagaagagcctctccctgtctctgggt

SEQ ID NO: 120 - DNA, 1347 bp, Artificial Sequence
  source: /note=“Description of Artificial Sequence: Synthetic polynucleotide”
gaagttcagctggttgaaagcggtggtggtctggttcagcctggtcgtagcctgcgtctgagctgtgcagcaagcggttttacctttgatgattatgccatgcattgggttcgtcaggcaccgggtaaaggtctggaatgggttagcggtattaattggagcagcggtggtattggttatgcagatagcgttaaaggtcgttttaccattagccgtgataatgccaaaaatagcctgtacctgcagatgaatagtctgcgtgcagaagataccgcactgtattattgtgcacgtgatattggtggttttggcgaattctattggaattttggtctgtggggtcgtggcaccctggttaccgttagcagcgcctccaccaagggcccatcggtcttccccctggcaccctgctcccgcagtacttctgagtccacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcaccaagacctacacgtgcaacgtggatcacaagcccagcaacaccaaggtggacaaacgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgagctgcgctgtcaaaggcttctaccccagcgacatcgccgtggagtgggagagcaatgggcagccggagaacaactacaagaccacgcctcccgtgctggactccgacggctccttcttcctcgttagcaggctaaccgtggacaagagcaggtggcaggaggggaatgtcttctcatgctccgtgatgcatgaggctctgcacaaccactacacacagaagagcctctccctgtctctgggt

SEQ ID NO: 121 - protein, 127 aa, Artificial Sequence
  source: /note=“Description of Artificial Sequence: Synthetic polypeptide”
QVHLQQSGAELAKPGASVHMSCKASGYTFTTYWMHWVKQRPGQGLEWIGYINPNTGYADYNQKFRDKATLTADKSSSTAYMQLSSLTSEDSTVYFCARRRTGTSYFDYWGQGTTLTVSSTKTTPPSV

SEQ ID NO: 122 - protein, 102 aa, Artificial Sequence
  source: /note=“Description of Artificial Sequence: Synthetic polypeptide”
QTVLSQSPAILSASPGEKVTMTCRASSSVTYMHWYQQKPGSSPKPWIFATSNLASGVPARFSGSGSGTSYSLTISRVEAEDAATYYCQQWTSNPLTFGAGTK

SEQ ID NO: 123 - protein, 127 aa, Artificial Sequence
  source: /note=“Description of Artificial Sequence: Synthetic polypeptide”
QVQLQQSGAELARPGASVKLSCKASGYSFTHYWMQWVKQRPGQGLEWIGAIYPGDGDTRYTQRLKGKATLTADKSSSTAYMELSSLASEDSAVYYCAREGEGNSAMDYWGQGTSVTVSSAKTTPPSV

SEQ ID NO: 124 - protein, 103 aa, Artificial Sequence
  source: /note=“Description of Artificial Sequence: Synthetic polypeptide”
DIVMTQSQKFMSTSVGDRVSVTCKASQNVGSSVAWFQQKPGQSPKTLIYSASYRDSGVPDRFTGSGSGTDFTLTITNVQSEDLTDYFCLQYGTYPYTFGGGTR